(19) United States
(12) Patent Application Publication
(10) Pub. No.: US 2015/0337378 A1
(43) Pub. Date: Nov. 26, 2015

(54) METHODS OF DIAGNOSIS AND TREATMENT OF INFLAMMATORY BOWEL DISEASE

(71) Applicant: CEDARS-SINAI MEDICAL CENTER, Los Angeles, CA (US)

(72) Inventors: Stephan R. Targan, Santa Monica, CA (US); Dermot P. McGovern, Los Angeles, CA (US); Ling Mei, Pasadena, CA (US); Jerome I. Rotter, Los Angeles, CA (US); Kent D. Taylor, Ventura, CA (US)

(73) Assignee: CEDARS-SINAI MEDICAL CENTER, Los Angeles, CA (US)

(21) Appl. No.: 14/722,018

(22) Filed: May 26, 2015

Related U.S. Application Data
(63) Continuation-in-part of application No. 12/528,668, filed on Aug. 26, 2009, now abandoned.
Application No. 12/528,668 is a continuation-in-part of application No. 12/598,794, filed on Nov. 4, 2009, now abandoned.
Application No. 12/598,794 is a continuation-in-part of application No. 13/121,929, filed on Mar. 30, 2011.
(60) Provisional application No. 60/892,165, filed on Feb. 28, 2007.
(60) Provisional application No. 60/916,026, filed on May 4, 2007.
(60) Provisional application No. 61/101,779, filed on Oct. 1, 2008.

(51) Int. Cl. C12Q 1/68

(52) U.S. Cl. CPC C12Q 1/6883; C12Q 2600/172; C12Q 2600/156

(57) ABSTRACT

The present invention relates to methods of diagnosing and predicting susceptibility to Crohn's Disease and/or IBD, by determining the presence or absence of susceptibility to genetic variants, risk haplotypes and/or protective haplotypes. In an embodiment, the invention provides methods of diagnosing and/or predicting susceptibility to Crohn's Disease in an individual by determining the presence or absence of risk variants at the IL12RB1, IL12RB2, IL17A, IL17RA, IL17RD and/or IL23R locus.

### CROSS-REFERENCE TO RELATED APPLICATIONS

This application is a continuation-in-part of U.S. Ser. No. 12/528,668 filed on Aug. 26, 2009, currently pending, which is a U.S. national stage application of PCT/US2008/055236 filed Feb. 28, 2008, now expired, which claims priority to U.S. Ser. No. 60/892,165 filed Feb. 28, 2007; and is a continuation-in-part of U.S. Ser. No. 12/598,794 filed on Nov. 4, 2009, currently pending, which is a national stage application of PCT/US2008/062531 filed May 2, 2008, now expired, which claims priority to U.S. Ser. No. 60/916,026 filed May 4, 2007 now expired; and is a continuation-in-part of U.S. Ser. No. 13/121,929 filed on Mar. 30, 2011, currently pending, which is a national stage application of PCT/US2009/059190 filed Oct. 1, 2009, now expired, which claims priority to U.S. Ser. No. 61/101,779 filed Oct. 1, 2008, the contents of each of which are herein incorporated by reference in their entirety.

### GOVERNMENT RIGHTS

This invention was made with government support under Grant Nos. DK046763 and DK066248 awarded by the National Institutes of Health. The U.S. Government may have certain rights in this invention.

### FIELD OF THE INVENTION

The invention relates generally to the fields of inflammation and autoimmunity and autoimmune disease and, more specifically, to genetic methods for diagnosing, predicting disease progression and treating inflammatory bowel disease, Crohn's disease, and other autoimmune diseases.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

The replicated associations between CD and variants in CARD15 and the IBD5 haplotype do not fully explain the genetic risk for CD. Thus, there is need in the art to determine other genes, allelic variants and/or haplotypes that may assist in explaining the genetic risk, diagnosing, and/or predicting susceptibility for or protection against inflammatory bowel disease including but not limited to CD and/or UC.

### SUMMARY OF THE INVENTION

Various embodiments of the present invention provides for methods of diagnosing susceptibility to Inflammatory Bowel Disease (IBD) in a subject in need thereof, comprising: providing a sample from the subject; assaying the sample to detect risk and/or protective haplotypes in one or more genes selected from the group consisting of: IL23R, IL17A, IL17RA, IL17RD, IL12B(p40), IL12RB1 and IL12RB2; and determining that the subject has an increased susceptibility to IBD if one or more risk haplotypes are present and the protective haplotypes are absent or determining that the subject has a decreased susceptibility to IBD if one or more protective haplotypes are present and the risk haplotypes are absent. In other embodiment, IBD comprises Crohn's Disease (CD) and ulcerative colitis (UC). In another embodiment, the risk haplotypes located at the: IL23R locus are IL23R Block 2 Haplotype 1 (B2H1) and/or IL23R Block 3 Haplotype 1 (B3H1); IL17A locus are IL17A Haplotype 2 (H2); IL17RA locus is IL17RA Block 2 Haplotype 4 (B2H4); IL17RD locus is IL17RD Block 2 Haplotype 2 (B2H2); IL12RB1 locus is IL12RB1 Haplotype 1 (H1); and IL12RB2 locus are IL12RB2 Haplotype 1 (H1) and/or IL12RB2 Haplotype 3 (H3). In other embodiment, the protective haplotypes located at the: IL23R locus are IL23R Block 2 Haplotype 2 (B2H2), IL23R Block 3 Haplotype 2 (B3H2) and/or IL23R Block 3 Haplotype 6 (B3H6); IL17A locus is IL17A Haplotype 4 (H4); IL17RA locus is IL17RA Block 1 Haplotype 3 (B1H3); IL17RD locus are IL17RD Block 1 Haplotype 2 (B1H2) and/or IL17RD Block 2 Haplotype 3 (B2H3); IL12B(p40) locus are IL12B(p40) Haplotype 1 (H1) and/or IL12B(p40) Haplotype 3 (H3); and IL12RB2 locus is IL12RB2 Haplotype 4 (H4).

In various other embodiments, the detection of IL23R B2H1 and/or IL23R B3H1 haplotypes in a pediatric non-Jewish subject indicates an increased susceptibility to IBD. In other embodiments, the detection of IL12RB2 H4 haplotype in a subject of Ashkenazi Jewish descent indicates a decreased susceptibility to IBD and wherein the detection of IL12B2H1 and/or IL12B2H3 haplotypes in a subject of Ashkenazi Jewish descent indicates an increased susceptibility to IBD. In other embodiments, the detection of IL17A H2 haplotype in a non-Jewish subject or the detection of IL17A H4 haplotype in a Jewish subject indicates an increased susceptibility to IBD. In other embodiments, the detection of IL12B(p40) H3 haplotype and Cbir1 antibody expression indicates a decreased susceptibility to UC in a subject.

In various other embodiments, the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP) and denaturing gradient gel electrophoresis (DGGE). In other embodiments, the detection of risk and/or protective haplotypes is relative to that detected in a healthy subject. In other embodiments, the presence of nine risk haplotypes presents a greater susceptibility than the presence of eight, seven, six, five, four, three, two, one or none of the risk haplotypes, and the presence of eight risk haplotypes presents a greater susceptibility than the presence of seven, six, five, four, three, two, one or none of the risk haplotypes, and the presence of seven risk haplotypes presents a greater susceptibility than the presence of six, five, four, three, two, one or none of the risk haplotypes, and the presence of six risk haplotypes presents a greater susceptibility than the presence of five, four, three, two, one or none of the risk haplotypes, and the presence of five risk haplotypes presents a greater susceptibility than the presence of four, three, two, one or none of the risk haplotypes, and the presence of four risk haplotypes presents a greater susceptibility than the presence of three, two, one or none of the risk haplotypes, and the presence of three risk haplotypes presents a greater susceptibility than the presence of two, one or none of the risk haplotypes, and the presence of two risk haplotypes presents a greater susceptibility than the presence of one or none of the risk haplotypes, and the presence of one risk haplotype presents a greater susceptibility than the presence of none of the risk haplotypes.

The invention also provides embodiments, for a process for selecting a therapy for a subject susceptible to IBD comprising providing a sample from the subject; assaying the sample to detect risk and/or protective haplotypes from genes selected from the group consisting of: IL23R, IL17A, IL17RA, IL17RD, IL12B(p40), IL12RB1 and IL12RB2; determining that the subject has an increased susceptibility to IBD if one or more risk haplotypes are present and the protective haplotypes are absent or determining that the subject has a decreased susceptibility to IBD if one or more protective haplotypes are present and the risk haplotypes are absent; and selecting a preventative therapy for a subject with an increased susceptibility to IBD. In other embodiments, IBD comprises Crohn's Disease (CD) and ulcerative colitis (UC). In another embodiment, the risk haplotypes located at the: IL23R locus are IL23R Block 2 Haplotype 1 (B2H1) and/or IL23R Block 3 Haplotype 1 (B3H1); IL17A locus are IL17A Haplotype 2 (H2); IL17RA locus is IL17RA Block 2 Haplotype 4 (B2H4); IL17RD locus is IL17RD Block 2 Haplotype 2 (B2H2); IL12RB1 locus is IL12RB1 Haplotype 1 (H1); and IL12RB2 locus are IL12RB2 Haplotype 1 (H1) and/or IL12RB2 Haplotype 3 (H3). In other embodiment, the protective haplotypes located at the: IL23R locus are IL23R Block 2 Haplotype 2 (B2H2), IL23R Block 3 Haplotype 2 (B3H2) and/or IL23R Block 3 Haplotype 6 (B3H6); IL17A locus is IL17A Haplotype 4 (H4); IL17RA locus is IL17RA Block 1 Haplotype 3 (B1H3); IL17RD locus are IL17RD Block 1 Haplotype 2 (B1H2) and/or IL17RD Block 2 Haplotype 3 (B2H3); IL12B(p40) locus are IL12B(p40) Haplotype 1 (H1) and/or IL12B(p40) Haplotype 3 (H3); and IL12RB2 locus is IL12RB2 Haplotype 4 (H4).

In various other embodiments, the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP) and denaturing gradient gel electrophoresis (DGGE). In other embodiments, the presence of ten risk haplotypes presents a greater susceptibility than the presence of nine, eight, seven, six, five, four, three, two, one or none of the risk haplotypes, and the presence of nine risk haplotypes presents a greater susceptibility than the presence of eight, seven, six, five, four, three, two, one or none of the risk haplotypes, and the presence of eight risk haplotypes presents a greater susceptibility than the presence of seven, six, five, four, three, two, one or none of the risk haplotypes, and the presence of seven risk haplotypes presents a greater susceptibility than the presence of six, five, four, three, two, one or none of the risk haplotypes, and the presence of six risk haplotypes presents a greater susceptibility than the presence of five, four, three, two, one or none of the risk haplotypes, and the presence of five risk haplotypes presents a greater susceptibility than the presence of four, three, two, one or none of the risk haplotypes, and the presence of four risk haplotypes presents a greater susceptibility than the presence of three, two, one or none of the risk haplotypes, and the presence of three risk haplotypes presents a greater susceptibility than the presence of two, one or none of the risk haplotypes, and the presence of two risk haplotypes presents a greater susceptibility than the presence of one or none of the risk haplotypes, and the presence of one risk haplotype presents a greater susceptibility than the presence of none of the risk haplotypes.

In other embodiments, the detection of risk and/or protective haplotypes is relative to that detected in a healthy subject.

Various embodiments of the present invention provides for a process for stratifying patients comprising: providing a sample from the subject; assaying the sample for risk and/or protective haplotypes from genes selected from the group consisting of: IL23R, IL17A, IL17RA, IL17RD, IL12B(p40), IL and IL12RB2; and a stratifying patients as having a low probability to develop IBD if an increased number of protective haplotypes and a decreased number of risk haplotypes are detected or stratifying patients as having a high probability to develop IBD if a decreased number of protective haplotypes and an increased number of risk haplotypes are detected. In other embodiments, IBD comprises Crohn's Disease (CD) and ulcerative colitis (UC).

In another embodiment, the risk haplotypes located at the: IL23R locus are IL23R Block 2 Haplotype 1 (B2H1) and/or IL23R Block 3 Haplotype 1 (B3H1); IL17A locus are IL17A Haplotype 2 (H2); IL17RA locus is IL17RA Block 2 Haplotype 4 (B2H4); IL17RD locus is IL17RD Block 2 Haplotype 2 (B2H2); IL12RB1 locus is IL12RB1 Haplotype 1 (H1); and IL12RB2 locus are IL12RB2 Haplotype 1 (H1) and/or IL12RB2 Haplotype 3 (H3). In other embodiment, the protective haplotypes located at the: IL23R locus are IL23R Block 2 Haplotype 2 (B2H2), IL23R Block 3 Haplotype 2 (B3H2) and/or IL23R Block 3 Haplotype 6 (B3H6); IL17A locus is IL17A Haplotype 4 (H4); IL17RA locus is IL17RA Block 1 Haplotype 3 (B1H3); IL17RD locus are IL17RD Block 1 Haplotype 2 (B1H2) and/or IL17RD Block 2 Haplotype 3 (B2H3); IL12B(p40) locus are IL12B(p40) Haplotype 1 (H1) and/or IL12B(p40) Haplotype 3 (H3); and IL12RB2 locus is IL12RB2 Haplotype 4 (H4).

In various other embodiments, the detection of IL23R B2H1 and/or IL23R B3H1 haplotypes in a pediatric non-Jewish subject indicates an increased probability for IBD. In other embodiments, the detection of IL12RB2 H4 haplotype in a subject of Ashkenazi Jewish descent indicates a decreased susceptibility to IBD and wherein the detection of IL12B2 H1 and/or IL12B2 H3 haplotypes in a subject of Ashkenazi Jewish descent indicates an increased susceptibility to IBD. In other embodiments, the detection of IL17A H2 haplotype in a non-Jewish subject and the detection of IL17A H4 haplotype in a Jewish subject indicates an increased probability for IBD.

In various other embodiments, the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP) and denaturing gradient gel electrophoresis (DGGE). In other embodiments, the presence of ten risk haplotypes presents a greater susceptibility than the presence of nine, eight, seven, six, five, four, three, two, one or none of the risk haplotypes, and the presence of nine risk haplotypes presents a greater susceptibility than the presence of eight, seven, six, five, four, three, two, one or none of the risk haplotypes, and the presence of eight risk haplotypes presents a greater susceptibility than the presence of seven, six, five, four, three, two, one or none of the risk haplotypes, and the presence of seven risk haplotypes presents a greater susceptibility than the presence of six, five, four, three, two, one or none of the risk haplotypes, and the presence of six risk haplotypes presents a greater susceptibility than the presence of five, four, three, two, one or none of the risk haplotypes, and the presence of five risk haplotypes presents a greater susceptibility than the presence of four, three, two, one or none of the risk haplotypes, and the presence of four risk haplotypes presents a greater susceptibility than the presence of three, two, one or none of the risk haplotypes, and the presence of three risk haplotypes presents a greater susceptibility than the presence of two, one or none of the risk haplotypes, and the presence of two risk haplotypes presents a greater susceptibility than the presence of one or none of the risk haplotypes, and the presence of one risk haplotype presents a greater susceptibility than the presence of none of the risk haplotypes.

In other embodiments, the detection of risk and/or protective haplotypes is relative to that detected in a healthy subject.

Various embodiments of the present invention provides for a method of diagnosing susceptibility of IBD in a pediatric non-Jewish subject comprising: providing a sample from the pediatric non-Jewish subject; assaying the sample for the risk haplotypes IL23R B2H1 and/or IL23R B3H1; and determining that the subject has an increased susceptibility to IBD if the risk haplotypes IL23R B2H1 and/or IL23R B3H1 are present or determining that the subject has a decreased susceptibility to IBD if the risk haplotypes IL23R B2H1 and/or IL23R B3H1 are absent. In other embodiments, IBD comprises Crohn's Disease (CD) and ulcerative colitis (UC). In other embodiments, the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP) and denaturing gradient gel electrophoresis (DGGE). In other embodiments, the detection of risk haplotypes is relative to that detected in a healthy subject. In other embodiments, the detection of risk haplotypes is relative to that detected in a healthy subject. In other embodiments, there is a greater susceptibility to IBD when an increased number of risk haplotypes are present and a decreased susceptibility when a decreased number of risk haplotypes are present.

Various embodiments of the present invention provides for a method of diagnosing susceptibility of IBD in subject of Ashkenazi Jewish descent comprising: providing a sample from the subject of Ashkenazi Jewish descent; assaying the sample for the risk haplotypes IL12B2H1 and/or IL12B2H3; and determining that the subject has an increased susceptibility to IBD if the risk haplotypes IL12B2H1 and/or IL12B2H3 are present or determining that the subject has a decreased susceptibility to IBD if the risk haplotypes IL12B2H1 and/or IL12B2H3 are absent. In other embodiments, IBD comprises Crohn's Disease (CD) and ulcerative colitis (UC). In other embodiments, the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP), denaturing gradient gel electrophoresis (DGGE). In other embodiments, the detection of risk haplotypes is relative to that detected in a healthy subject. In other embodiments, there is a greater susceptibility to IBD when an increased number of risk haplotypes are present and a decreased susceptibility when a decreased number of risk haplotypes are present.

Various embodiments provide methods of diagnosing susceptibility to Crohn's Disease in an individual, comprising determining the presence or absence of at least one risk haplotype at the IL23R locus selected from the group consisting of IL23R Block 2 H1 and IL23R Block 3 H1, where the presence of at least one risk haplotype at the IL23R locus is diagnostic of susceptibility to Crohn's Disease. In another embodiment, the individual may be a child and/or non-Jewish.

In another embodiment, the IL23R Block 2 H1 haplotype further comprises one or more variant alleles selected from the group consisting of SEQ ID NO: 9 and SEQ ID NO: 10. In another embodiment, the IL23R Block 3 H1 haplotype further comprises one or more variant alleles selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17 and SEQ ID NO: 18. In another embodiment, the presence of two of the risk haplotypes at the IL23R locus presents a greater susceptibility than the presence of one or none of the risk haplotypes at the IL23R locus, and the presence of one of the risk haplotypes at the IL23R locus presents a greater susceptibility than the presence of none of the risk haplotypes at the IL23R locus but less than the presence of the two risk haplotypes at the IL23R locus.

Other embodiments provide methods of diagnosing susceptibility to Crohn's Disease in an individual, comprising determining the presence or absence of one or more risk haplotypes at the IL23R locus, and determining the presence or absence of one or more risk haplotypes at the IL17A locus, where the presence of at least one risk haplotype at the IL23R locus and at least one risk haplotype at the IL17A locus is diagnostic of susceptibility of Crohn's Disease. In other embodiments, one of the one or more risk haplotypes at the IL23R locus may be IL23R Block 2 H1, and/or IL23R Block 3 H1. In another embodiment, one of the one or more risk haplotypes at the IL17A locus is IL17A H2. The IL17A H2 may further comprise one or more variant alleles selected from the group consisting of SEQ ID NO: 19, SEQ ID NO: 20, SEQ ID NO: 21, SEQ ID NO: 22 and SEQ ID NO: 23.

Various embodiments provide methods of diagnosing susceptibility to Crohn's Disease in an individual, comprising determining the presence or absence of at least one risk haplotype at the IL23R locus, and determining the presence or absence of at least one risk haplotype at the IL17RA locus, where the presence of at least one risk haplotype at the IL23R locus and at least one risk haplotype at the IL17RA locus is diagnostic of susceptibility of Crohn's Disease. In other embodiments, one of the one or more risk haplotypes at the IL23R locus is IL23R Block 2 H1 and/or IL23R Block 3 H1. In other embodiments, one of the one or more risk haplotypes at the IL17RA locus is IL17RA Block 2 H4. The IL17RA Block 2 H4 may further comprise one or more variant alleles selected from the group consisting of SEQ ID NO: 26, SEQ ID NO: 27, SEQ ID NO: 28, SEQ ID NO: 29, SEQ ID NO: 30, SEQ ID NO: 31 and SEQ ID NO: 32.

Other embodiments provide methods of determining a low probability relative to a healthy individual of developing Crohn's Disease in an individual, the method comprising determining the presence or absence of at least one protective haplotype at the IL23R locus selected from the group consisting of IL23R Block 3 H2 and IL23R Block 3 H6, where the presence of one or more of the protective haplotypes at the IL23R locus is diagnostic of the low probability relative to the healthy individual of developing Crohn's Disease. In other embodiments, the IL23R Block 3 H2 further comprises one or more variant alleles selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17 and SEQ ID NO: 18. In other embodiments, the IL23R Block 3 H6 further comprise one or more variant alleles selected from the group consisting of SEQ ID NO: 11, SEQ ID NO: 12, SEQ ID NO: 13, SEQ ID NO.: 14, SEQ ID NO: 15, SEQ ID NO: 16, SEQ ID NO: 17 and SEQ ID NO: 18.

The invention also provides embodiments of methods of diagnosing susceptibility to Crohn's Disease in an individual, comprising determining the presence or absence of one or more risk haplotypes at the IL17A locus in the individual, where the presence of one or more of the risk haplotypes is diagnostic of susceptibility to Crohn's Disease. One of the one or more risk haplotypes at the IL17A locus may be IL17A H2. In other embodiments, the individual is non-Jewish. In other embodiments, one of the one or more risk haplotypes at the IL17A locus may be IL17A H4. In other embodiments, the individual is Jewish.

Various embodiments provide methods of diagnosing susceptibility to inflammatory bowel disease in an individual, comprising determining the presence or absence of one or more risk haplotypes at the IL17RA locus in the individual, where the presence of one or more of said risk haplotypes is diagnostic of susceptibility to inflammatory bowel disease. One of the one or more risk haplotypes at the IL17RA locus may be IL17RA Block 2 H4. The inflammatory bowel disease may also comprise Crohn's Disease and/or ulcerative colitis.

Other embodiments provide methods of determining a low probability relative to a healthy individual of developing inflammatory bowel disease in an individual, the method comprising determining the presence or absence of one or more protective haplotypes at the IL17RA locus in the individual, where the presence of one or more of said protective haplotypes is diagnostic of the low probability relative to the healthy individual of developing inflammatory bowel disease. One of the one or more protective haplotypes at the IL17RA locus may be IL17RA Block 1 H3. The inflammatory bowel disease may also comprise Crohn's Disease and/or ulcerative colitis.

Various embodiments also provide methods of determining a low probability relative to a healthy individual of developing Crohn's Disease subtype, comprising determining the presence or absence of a IL12B(p40) H1 haplotype, where the presence of the IL12B(p40) H1 haplotype is diagnostic of a low probability relative to a healthy individual of developing Crohn's Disease. The IL12B(p40) H1 haplotype may also further comprise one or more variant alleles selected from the group consisting of SEQ ID NO: 33, SEQ ID NO: 34, SEQ ID NO: 35 and SEQ ID NO: 36.

Embodiments provide for methods of diagnosing a low probability relative to a healthy individual of developing Crohn's Disease, comprising determining the presence or absence of a IL12B(p40) H3 haplotype, and determining the presence or absence of Cbir1 antibody expression relative to an individual diagnosed with Crohn's Disease, where the presence of IL12B(p40) H3 haplotype and the absence of Cbir1 antibody expression relative to an individual diagnosed with Crohn's Disease is diagnostic of a low probability relative to a healthy individual of developing Crohn's Disease. The IL12B(p40) H3 haplotype may further comprise one or more variant alleles selected from the group consisting of SEQ ID NO: 33, SEQ ID NO: 34, SEQ ID NO: 35 and SEQ ID NO: 36.

Other embodiments provide methods of treating Crohn's Disease, comprising determining the presence or absence in the individual of one or more risk haplotypes selected from the group consisting of IL23R Block 2 H1, IL23R Block 3 H1, IL17A H2 and IL17RA Block 2 H4, and administering a therapeutically effective amount of treatment to the individual if the one or more risk haplotypes are present.

Various embodiments provide methods of diagnosing susceptibility to Crohn's Disease in an individual, comprising determining the presence or absence of a first risk haplotype at the IL23R locus, the presence or absence of a second risk haplotype at the IL17A locus, the presence or absence of a third risk haplotype at the IL17RA locus, and the presence or absence of a fourth risk haplotype at the IL12RB1 locus, where the presence of four of the risk haplotypes present a greater susceptibility than the presence of three, two, one or none of the risk haplotypes, and the presence of three risk haplotypes presents a greater susceptibility than the presence of two, one or none of the risk haplotypes, and the presence of two risk haplotypes presents a greater susceptibility than the presence of one or none of the risk haplotypes, and the presence of one of the risk haplotypes presents a greater susceptibility than the presence of none of the risk haplotypes. In another embodiment, the first risk haplotype at the IL23R locus comprises IL23R Block 2 H1 and/or Block 3 H1. In another embodiment, the first risk haplotype at the IL23R locus comprises a variant selected from the group consisting of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 37. In another embodiment, the second risk haplotype at the IL17A comprises IL17A H2. In another embodiment, the second risk haplotype at the IL17A locus comprises a variant selected from the group consisting of SEQ ID NO: 19, SEQ ID NO: 20, SEQ ID NO: 21 and SEQ ID NO: 22, SEQ ID NO: 23. In another embodiment, the third risk haplotype at the IL17RA locus comprises IL17RA Block 2 H4. In another embodiment, the third risk haplotype at the IL17RA locus comprises a variant selected from the group consisting of SEQ ID NO: 27, SEQ ID NO: 28, SEQ ID NO: 29, SEQ ID NO: 30, SEQ ID NO: 31 and SEQ ID NO: 32. In another embodiment, the fourth risk haplotype at the IL12RB1 locus comprises IL12RB1 H1. In another embodiment, the fourth risk haplotype at the IL12RB1 locus comprises a variant selected from the group consisting of SEQ ID NO: 38 and SEQ ID NO: 39.

Other embodiments provide methods of treating Crohn's Disease, comprising determining the presence of one or more risk haplotypes at the IL12RB1 locus, and treating the Crohn's Disease. In another embodiment, one of said one or more risk haplotypes at the IL12RB1 locus comprises SEQ ID NO: 38 and SEQ ID NO: 39.

Other embodiments provide methods of determining a low probability relative to a healthy subject of developing Crohn's Disease, comprising determining the presence or absence of a protective haplotype at the IL12RB2 locus in the individual, and diagnosing a low probability of developing Crohn's Disease, relative to a healthy subject, based upon the presence of the protective haplotype at the IL12RB2 locus. In other embodiments, the protective haplotype at the IL12RB2 locus comprises IL12RB2 H4. In other embodiments, the protective haplotype at the IL12RB2 locus comprises SEQ ID NO: 40, SEQ ID NO: 41 and SEQ ID NO: 42. In other embodiments, the individual is Ashkenazi Jewish.

Various other embodiments provide methods of diagnosing susceptibility to Crohn's Disease in an individual, comprising determining the presence or absence of one or more risk haplotypes at the IL12RB2 locus in the individual, and diagnosing susceptibility to Crohn's Disease based upon the presence of one or more risk haplotypes at the IL12RB2 locus. In other embodiments, one of said one or more risk haplotypes at the IL12RB2 locus is H3. In other embodiments, one of said one or more risk haplotypes at the IL12RB2 locus is H1. In other embodiments, the individual is Ashkenazi Jewish. In other embodiments, one of said one or more risk haplotypes at the IL12RB2 locus comprises SEQ ID NO: 40, SEQ ID NO: 41 and SEQ ID NO: 42.

Other embodiments provide methods of treating Crohn's Disease, comprising determining the presence of one or more risk haplotypes at the IL12RB2 locus, and treating the Crohn's Disease. In other embodiments, one of said one or more risk haplotypes at the IL12RB2 locus comprises SEQ ID NO: 40, SEQ ID NO: 41 and SEQ ID NO: 42.

Various embodiments include a method for diagnosing susceptibility to Crohn's disease in an individual, comprising determining the presence or absence of a risk haplotype at the IL17RD genetic locus in the individual, and diagnosing susceptibility to Crohn's disease in the individual based upon the presence of the risk haplotype at the IL17RD genetic locus. In another embodiment, the risk haplotype at the IL17RD genetic locus comprises IL17RD Block 2 Haplotype 2. In another embodiment, the risk haplotype at the IL17RD genetic locus comprises SEQ ID NO: 52, SEQ ID NO: 53 and/or SEQ ID NO: 54.

Other embodiments include a method for diagnosing susceptibility to Crohn's disease in an individual, comprising obtaining a sample from the individual, assaying the sample to determine the presence or absence of a risk haplotype at the IL17RD genetic locus in the individual, and diagnosing susceptibility to Crohn's Disease in the individual based upon the presence of the risk haplotype at the IL17RD genetic locus in the sample. In another embodiment, the risk haplotype at the IL17RD genetic locus comprises IL17RD Block 2 Haplotype 2. In another embodiment, assaying the sample comprises genotyping for one or more single nucleotide polymorphisms.

Other embodiments include a method of determining a low probability of developing Crohn's disease in an individual, relative to a healthy subject, comprising obtaining a sample from the individual, assaying the sample to determine the presence or absence of one or more protective haplotypes at the IL17RD genetic locus in the individual, and diagnosing a low probability of developing Crohn's disease in the individual, relative to a healthy subject, based upon the presence of one or more protective haplotypes at the IL17RD genetic locus. In another embodiment, the one or more protective haplotypes at the IL17RD genetic locus comprises IL17RD Block 1 Haplotype 2 and/or IL17RD Block 2 Haplotype 3. In another embodiment, the one or more protective haplotypes at the IL17RD genetic locus comprises SEQ ID NO: 55, SEQ ID NO: 56, SEQ ID NO: 57, SEQ ID NO: 58, SEQ ID NO: 59, and/or SEQ ID NO: 60. In another embodiment, the one or more protective haplotypes at the IL17RD genetic locus comprises SEQ ID NO: 52, SEQ ID NO: 53 and/or SEQ ID NO: 54. In another embodiment, assaying the sample comprises genotyping for one or more single nucleotide polymorphisms. In another embodiment, assaying the sample comprises specific hybridization of genomic DNA to arrayed probes.

Other embodiments include a method of diagnosing susceptibility to Crohn's disease in an individual, comprising obtaining a sample from the individual, assaying the sample for the presence or absence in the individual of a risk haplotype at the IL17RD genetic locus, a risk haplotype at the IL23R genetic locus, and a risk haplotype at the IL12RB2 genetic locus, and diagnosing susceptibility to Crohn's disease in the individual based upon the presence of the risk haplotype at the IL17RD genetic locus, the risk haplotype at the IL23R genetic locus, and the risk haplotype at the IL12RB2 genetic locus. In another embodiment, the risk haplotype at the IL23R genetic locus comprises IL23R Block 2 Haplotype 2. In another embodiment, the risk haplotype at the IL12RB2 genetic locus comprises IL12RB2 Haplotype 4. In another embodiment, the risk haplotype at the IL12RB2 genetic locus comprises SEQ ID NO: 40, SEQ ID NO: 41 and/or SEQ ID NO: 42. In another embodiment, assaying the sample comprises performing a whole-genome microarray assay. In another embodiment, assaying the sample comprises multidimensionality reduction.

Other features and advantages of the invention will become apparent from the following detailed description, taken in conjunction with the accompanying drawings, which illustrate, by way of example, various embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A-1B depicts a table of results from Transmission Distortion Test, used to test association to disease. 1A Depicts results from a Study Family Population; 1B Depicts results from a Pediatric Population.

FIG. 2 depicts chromosome 1 and IL23R SNPs and positions.

FIG. 3 depicts a graph of an example of SNPs associated with Crohn's Disease. Eight IL23R SNPs were ultimately found to be associated with Crohn's Disease and this is a graph demonstrating an example of this, comparing Crohn's Disease vs. Control for markers rs1343151 and rs11209026.

FIG. 4 depicts the SNPs, alleles, and positions of markers and three haplotype blocks observed in IL23R.

FIG. 5 depicts IL23R haplotype analysis. Block 2 is further described with corresponding haplotypes, nucleotides, and positions on chromosome.

FIG. 6 depicts IL23R haplotype analysis. Block 2 is further described, with a graph demonstrating H1 “risk” and H2 “protective” association for Crohn's Disease.

FIG. 7 depicts a chart demonstrating Crohn's Disease risk for IL23R Block 2 haplotypes.

FIG. 8 depicts a chart further describing SNPs, alleles, and positions of markers and haplotypes in Block 3 of IL23R.

FIG. 9 depicts a graph further describing Block 3 of IL23R, demonstrating H1 “risk” and H2 “protective” and H6 “protective” association for Crohn's Disease.

FIG. 10 depicts a chart demonstrating Crohn's Disease risk for IL23R Block 3 haplotypes.

FIG. 11 depicts a chart demonstrating IL23R haplotype combinations are associated with Crohn's Disease.

FIG. 12 depicts population attributable risk. The chart describes haplotypes of IL23R block 2, block 3, and both.

FIG. 13 depicts a chart of IL23R risk haplotypes. The chart describes both IL23R block 2 and 3 in correlation with 12 antibody expression levels.

FIG. 14 depicts haplotype structure of IL17A and haplotype frequencies.

FIG. 15 depicts a chart of IL17A in non-Jewish individuals with Crohn's Disease. The chart demonstrates IL17A H2 “risk” association and IL17A H4 “protective” association with Crohn's Disease.

FIG. 16 depicts a chart of IL17A diplotypes in non-Jewish Crohn's Disease, with diplotype equaling pairs of haplotypes, which in turn equaling haplogenotype.

FIG. 17 depicts the haplotype structure of IL17RA and haplotype frequencies.

FIG. 18 depicts IL17RA in combined Crohn's Disease and ulcerative colitis. The chart depicts IL17RA block 2 H4 “risk” association and IL17RA block 1 H3 “protective” association with IBD.

FIG. 19 depicts a chart of IL17RA haploblocks in combined Crohn's Disease and ulcerative colitis.

FIG. 20 depicts a graph of IL17A in Jewish and non-Jewish subgroups. The chart describes IL17A H4 “protective” and H2 “risk” association for non-Jewish Crohn's Disease patients, and IL17A H2 “protective” association for Jewish Crohn's Disease patients.

FIG. 21 depicts a chart of haplotype defined gene-gene interactions. The chart demonstrates the presence of synergy between IL23R and IL17A, and the presence of synergy between IL23R and IL17RA.

FIG. 22 depicts a chart demonstrating a lack of synergistic effect between IL17A and IL17RA in terms of gene-gene interactions.

FIG. 23 depicts a chart of the combined effect of IL23R, IL17A, and IL17RA, as demonstrated by plots of no risk haplotype, one risk haplotype, two risk haplotype, and three risk haplotype.

FIG. 24 depicts the IL12B haplotype structure, as well as a chart of haplotype frequency.

FIG. 25 depicts a graph of the association between IL12B haplotype and Crohn's Disease.

FIG. 26 depicts a graph of the association between IL12B and the presence of Anti-Cbir1.

FIG. 27 depicts a graph of the association between IL12B H3 and Anti-Cbir1 level.

FIG. 28 depicts a chart of haplotype defined gene-gene interactions. The chart demonstrates no synergistic effects between IL12B and IL23R protective haplotypes.

FIG. 29 depicts a chart of risk haplotype defined gene-gene interactions of IL17A, IL17RA, and IL23R.

FIG. 30 depicts a chart of protective haplotype defined gene-gene interactions of IL17A, IL17RA, and IL23R with IL12B.

FIG. 31A-31B (prior art) depicts the IL23/IL17 pathway. Sketch of the basic protein components of the IL23/IL17 pathway, leading to the development of the Th17 cell and subsequent production of IL17, contrasted with the IL12 pathway, leading to the development of the Th1 cell. Redrawn after Weaver, 2007. 31A The IL12 pathway; 31B the IL23/IL17 pathway.

FIG. 32 depicts the odds ratio for Crohn's disease with number of risk haplotypes. Odds ratio for CD for the presence of 0, 1, 2, 3, or 4 risk haplotypes for IL23R, IL17A, IL17RA, and IL12RB1.

FIG. 33A-33G depicts HapMap Data for Control Population, and observed structure of genes from association studies. 33A Observed IL23R Structure; 33B Observed IL17A Structure; 33C Observed IL17RA Structure; 33D Observed IL12B Structure; 33E Observed IL12RB1 Structure; 33F Observed IL12A Structure; 33G Observed IL12RB2 Structure.

FIG. 34 (4 pages) depicts a table listing the association of IL17-IL23 pathway related haplotypes with Crohn's Disease. With the exception of IL23R H6 which contains the R381Q variant, haplotypes with frequency >5% are shown. Variants are reported as the nucleotide on the forward strand of the NCBI Genome Build 36 and dbSNP v 126, although as would be obvious to one of skill in the art, the results described herein apply also to the complementary reverse strand.

FIG. 35 (3 pages) depicts, in accordance with an embodiment herein, association of IL17-IL23 pathway-related haplotypes with Crohn's Disease.

FIG. 36 depicts, in accordance with an embodiment herein, interaction between IL23R risk haplotypes and IL17A risk haplotype in non-Jewish subjects.

FIG. 37 depicts, in accordance with an embodiment herein, IL17RD haplotypes.

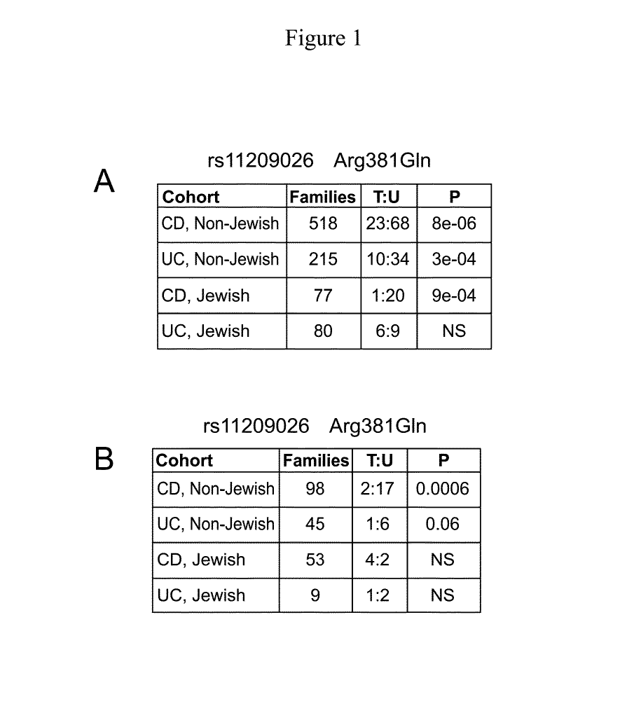

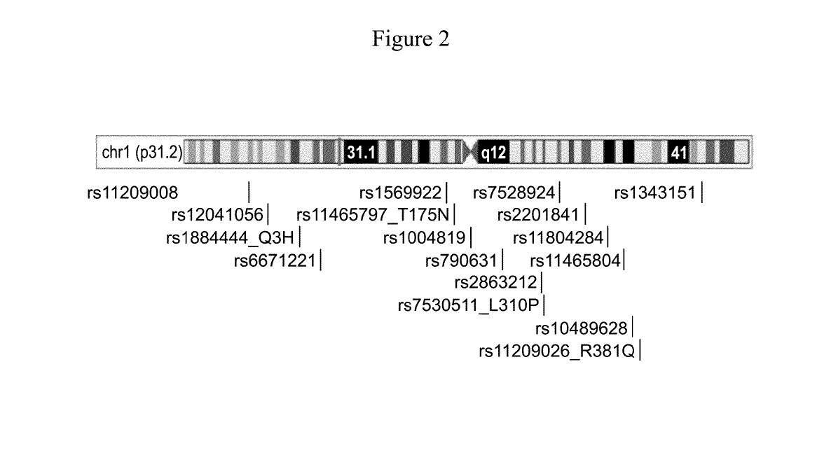

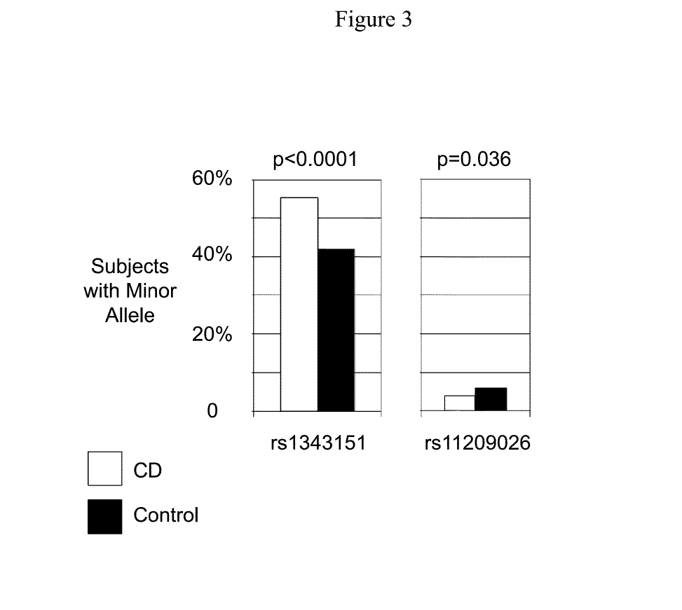

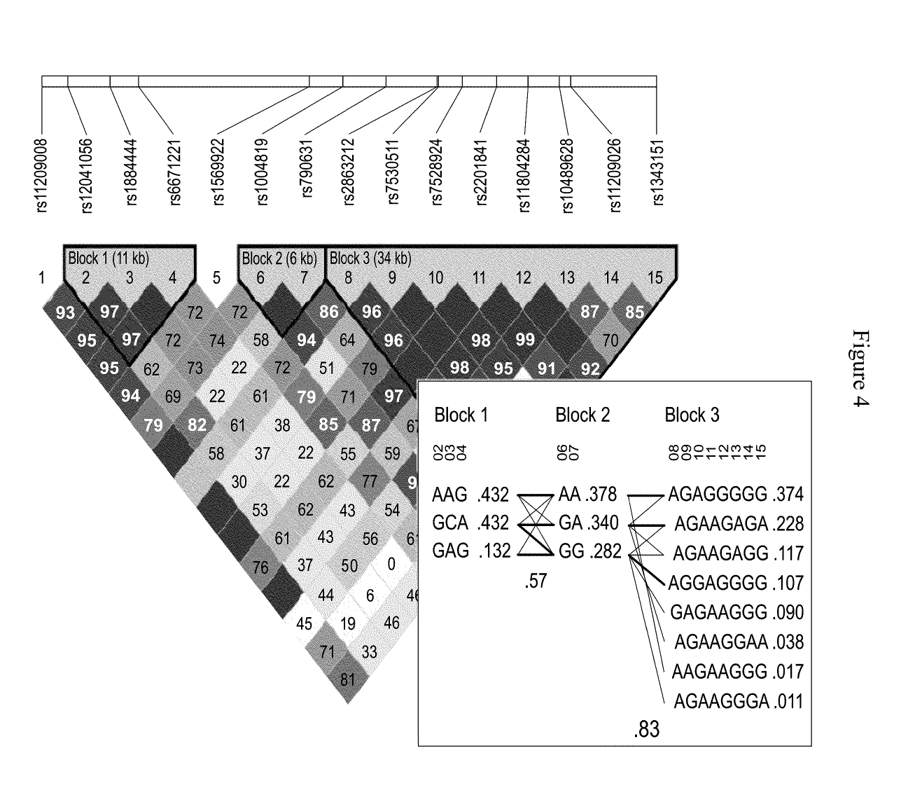

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“SNP” as used herein means single nucleotide polymorphism.

“Haplotype” as used herein refers to a set of SNPs on a gene or chromatid, with a combination of specific alleles that are statistically associated.

“Risk haplotype” as used herein refers to a haplotype whose presence is associated with an increase in susceptibility to an inflammatory bowel disease, relative to an individual who does not have the risk haplotype.

“Protective haplotype” as used herein refers to a haplotype whose presence is associated with a low probability relative to a healthy individual of developing inflammatory bowel disease. The protective haplotype is more frequently present in healthy individuals compared to individuals diagnosed with inflammatory bowel disease.

“Risk variant” as used herein refers to an allele, whose presence is associated with an increase in susceptibility to an inflammatory bowel disease, including but not limited to Crohn's Disease and ulcerative colitis, relative to an individual who does not have the risk variant.

“Protective variant” as used herein refers to an allele whose presence is associated with a low probability relative to a healthy individual of developing inflammatory bowel disease. The protective variant is more frequently present in healthy individuals compared to individuals diagnosed with inflammatory bowel disease.

“Protective” and “protection” as used herein refer to a decrease in susceptibility to IBD, including but not limited to CD and UC.

“Risk” as used herein refers to an increase in susceptibility to IBD, including but not limited to CD and UC.

“CD” and “UC” as used herein refer to Crohn's Disease and Ulcerative colitis, respectively.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

As used herein, “positive seroreactivity” means a high level of expression for an antibody relative to levels that would be found in a healthy individual. For example, determining the presence of Cbir1 antibody expression means that there is a high expression level of the Cbir1 antibody relative to the levels that would be found in a healthy individual. Conversely, determining the absence of Cbir1 antibody expression means that there is a low expression level of the Cbir1 antibody relative to the levels that would be found in a diseased individual.

As used herein, an “interaction” of genetic variants for conferring susceptibility to a disease is defined as an additive effect for the variants' association with susceptibility to the disease, so that the genetic variants are not independently associated with the disease. For example, in the case of an interaction determined to exist between two risk haplotypes, the presence of the two risk haplotypes would be determined to confer a greater susceptibility to the disease than would the presence of only one or none of the risk haplotypes.

As used herein, the abbreviation “IL12A” means interleukin 12A, “IL12B” means interleukin 12B, “IL12RB1” means interleukin 12 receptor beta 1, “IL12RB2” means interleukin 12 receptor beta 2, “IL17A” means interleukin 17A, “IL17RA” means interleukin 17 receptor A, IL17RD″ means interleukin 17 receptor D, “IL23R” means interleukin 23 alpha subunit p19, “IL23R” means interleukin 23 receptor.

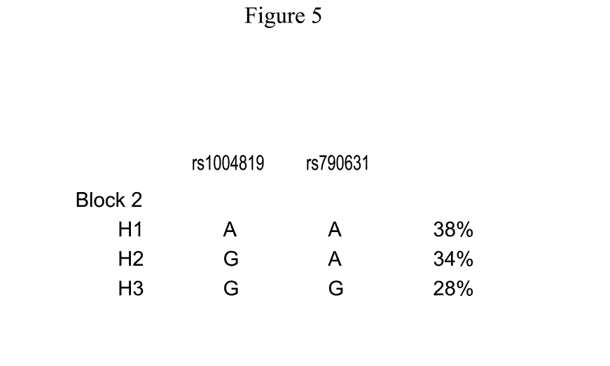

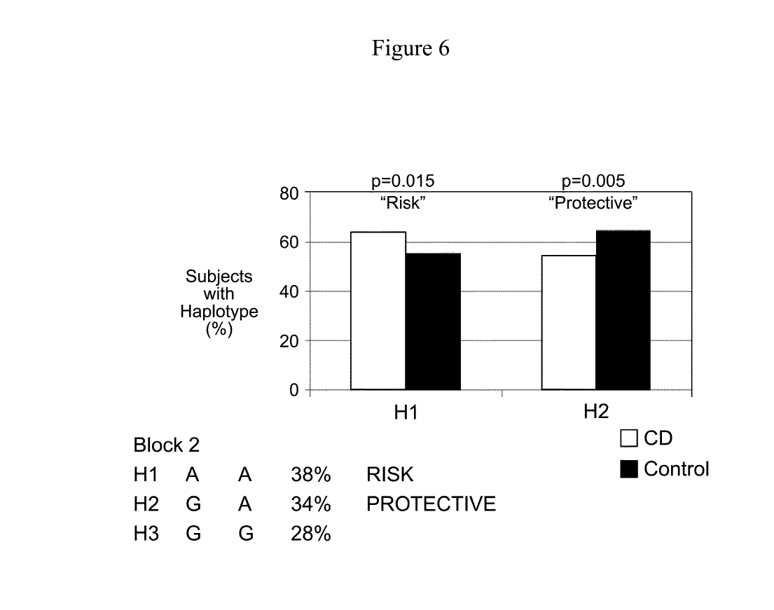

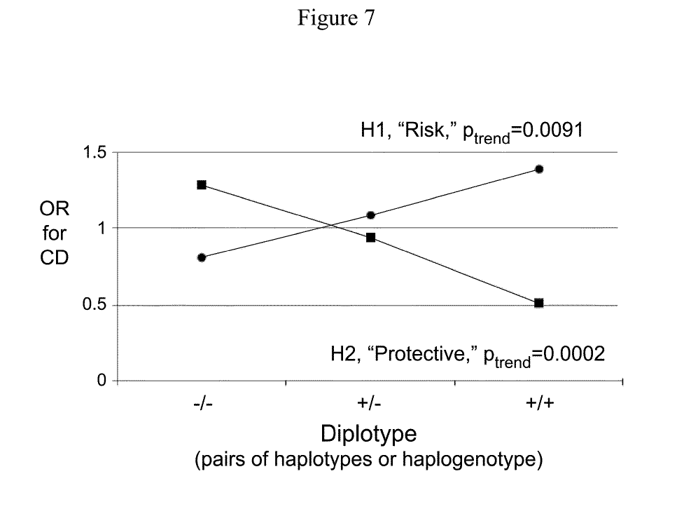

The identities of the IL23R Block 2 markers, their location on the gene and their nucleotide substitutions may be found in FIGS. 4-6.

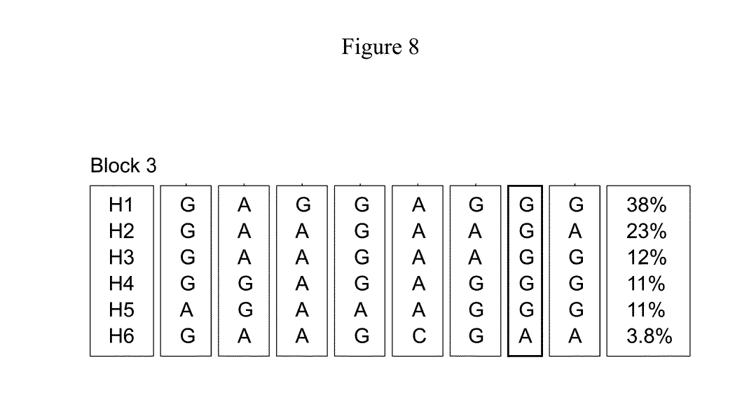

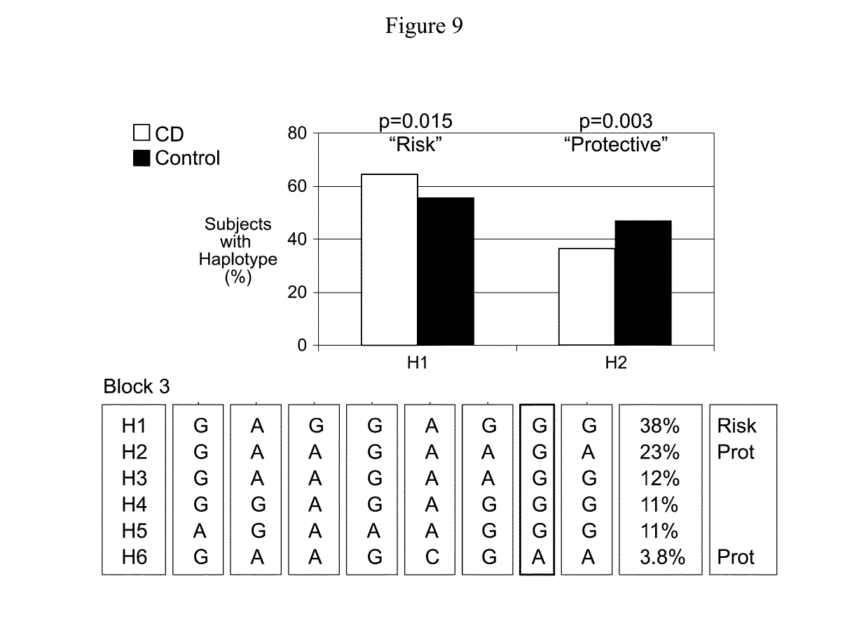

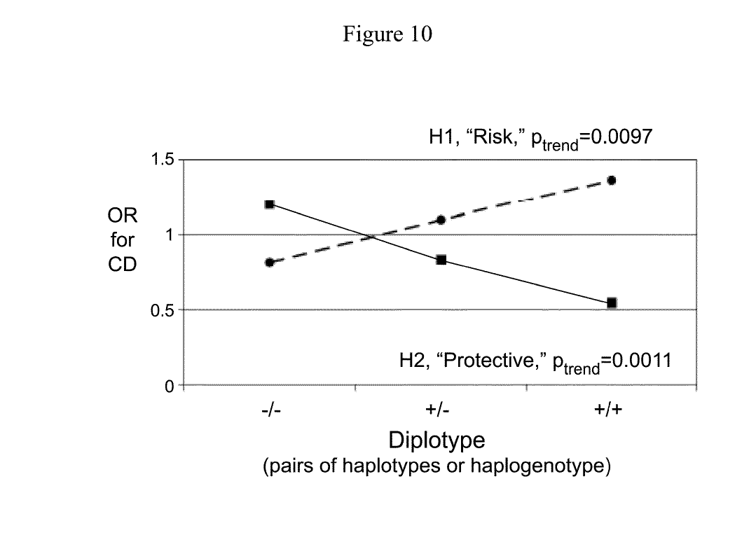

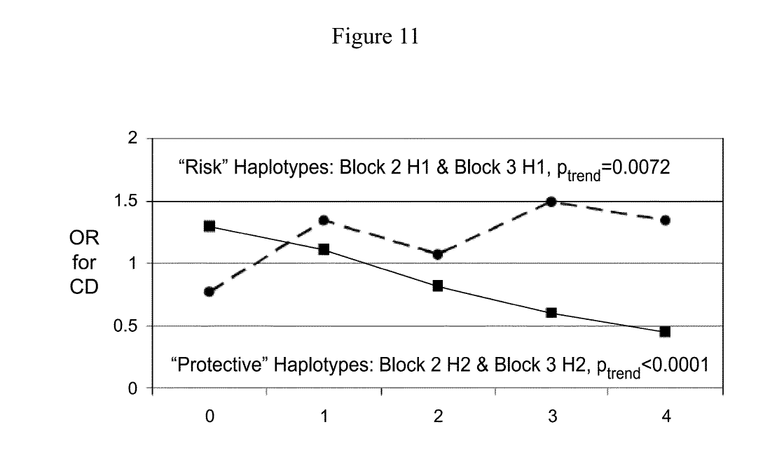

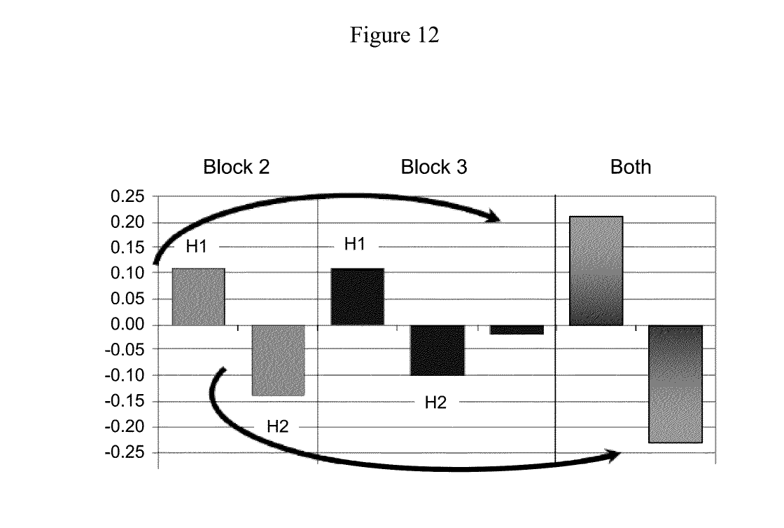

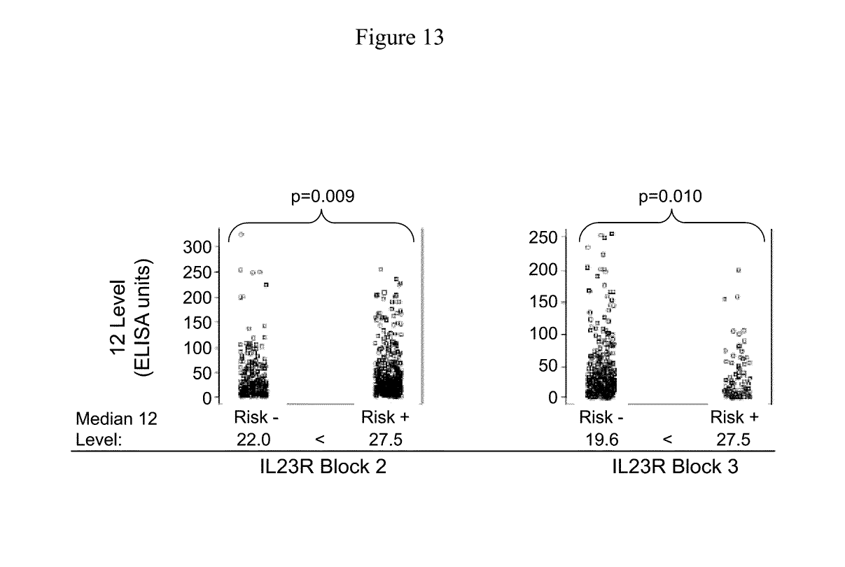

The identities of the IL23R Block 3 markers, their location on the gene and their nucleotide substitutions may be found in FIGS. 4 and 8-9.

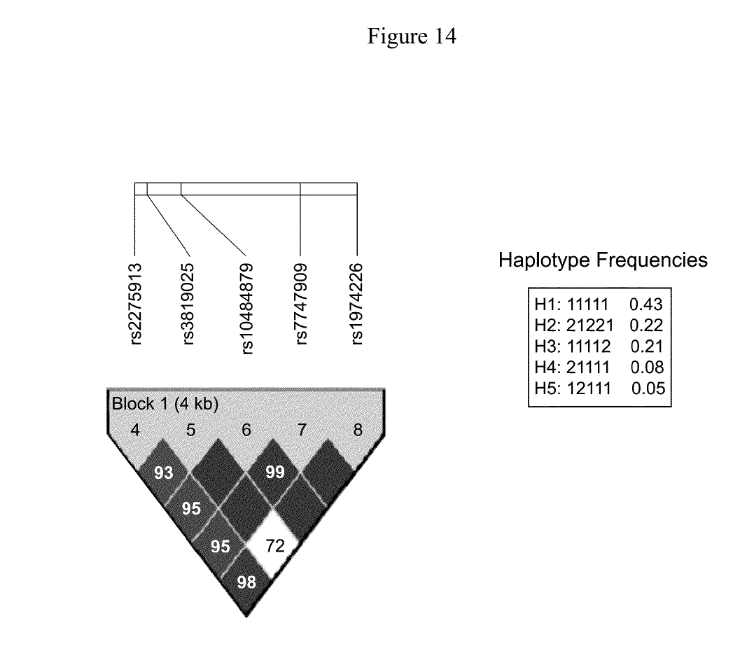

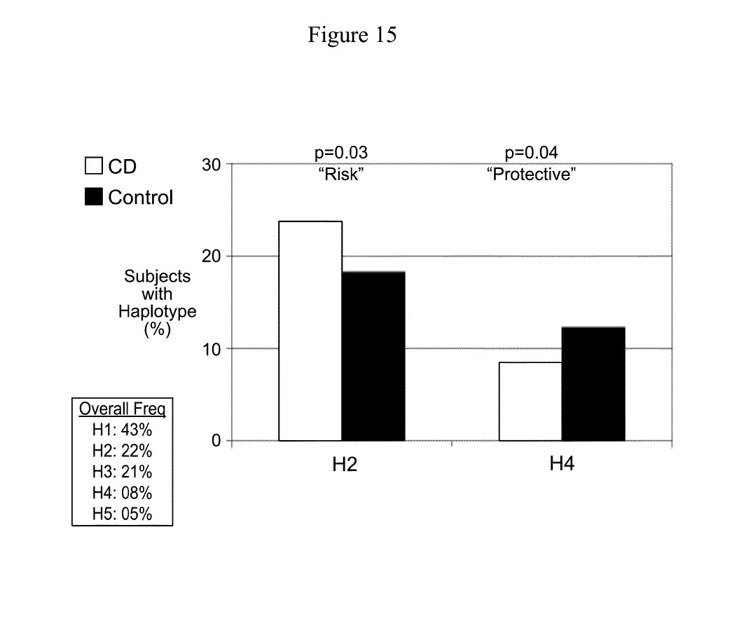

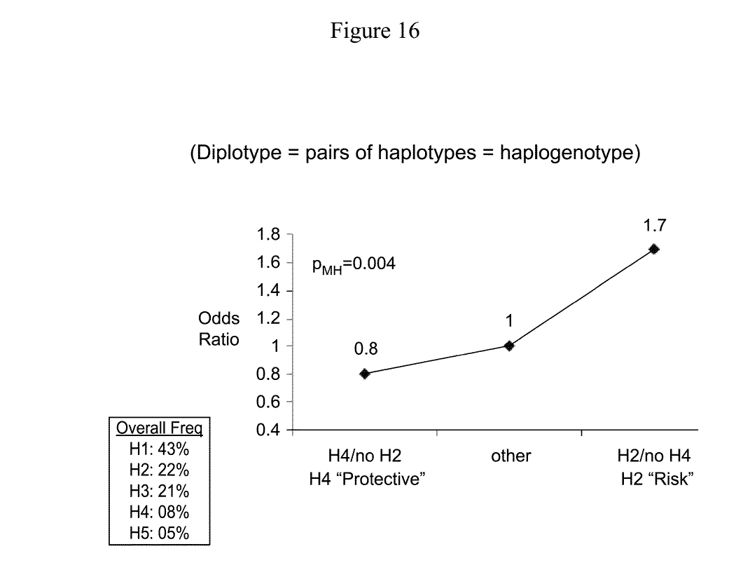

The identities of the IL17A markers, their location on the gene and their nucleotide substitutions may be found in Table 2, as well as FIG. 14.

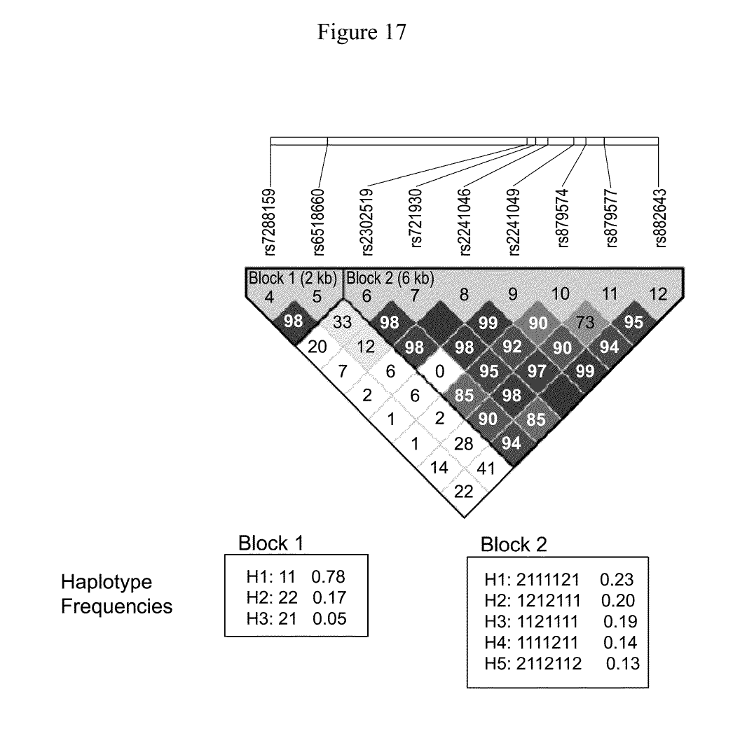

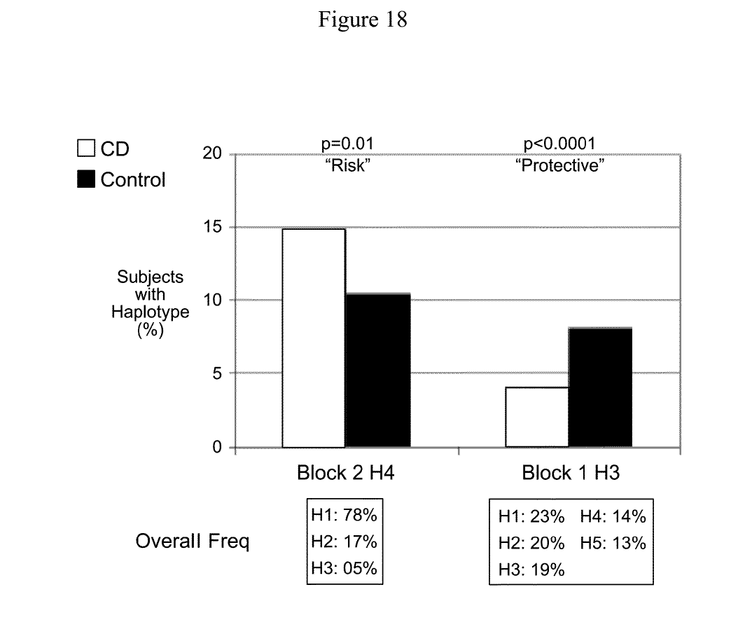

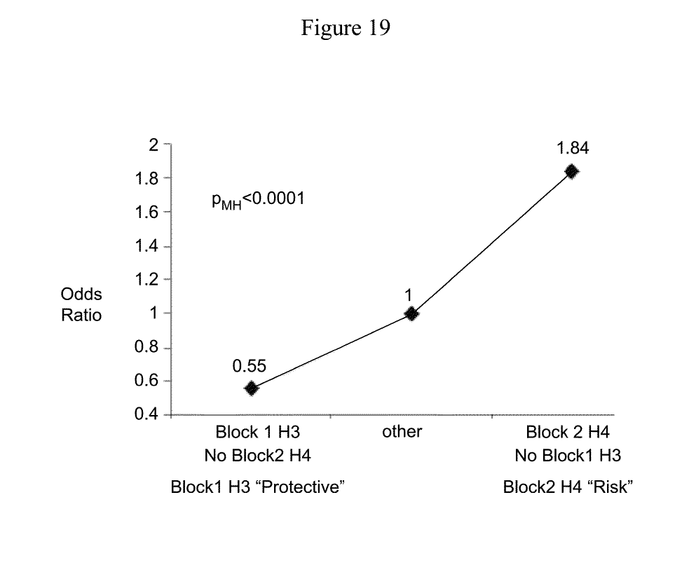

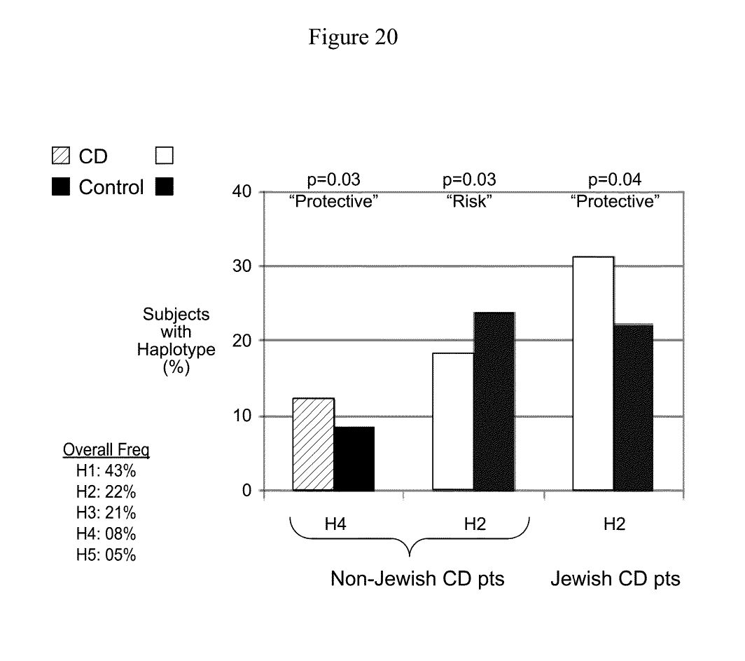

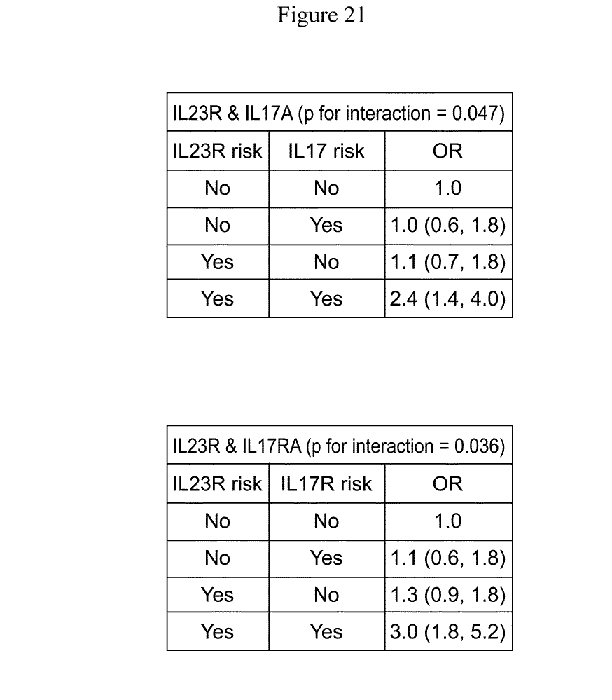

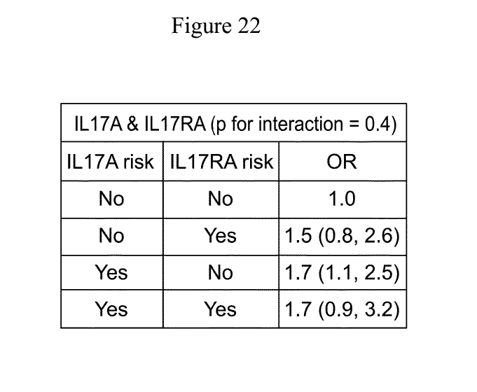

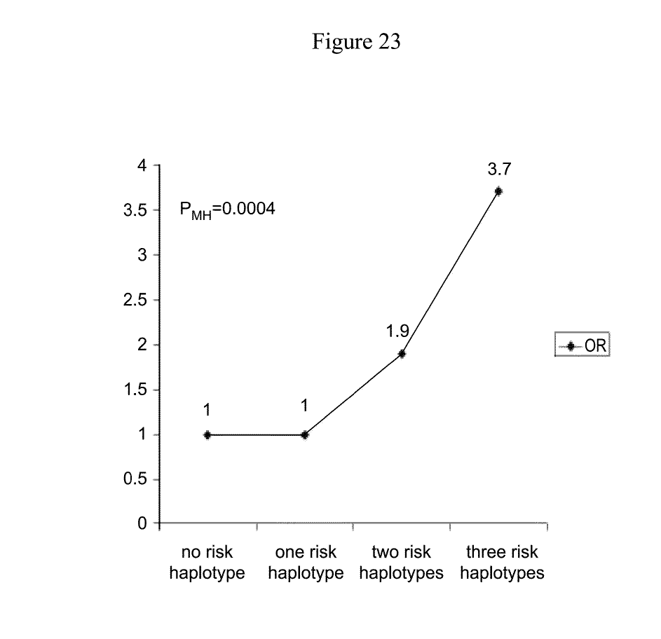

The identities of the IL17RA markers, their location on the gene and their nucleotide substitutions may be found in Table 3, as well as FIG. 17.

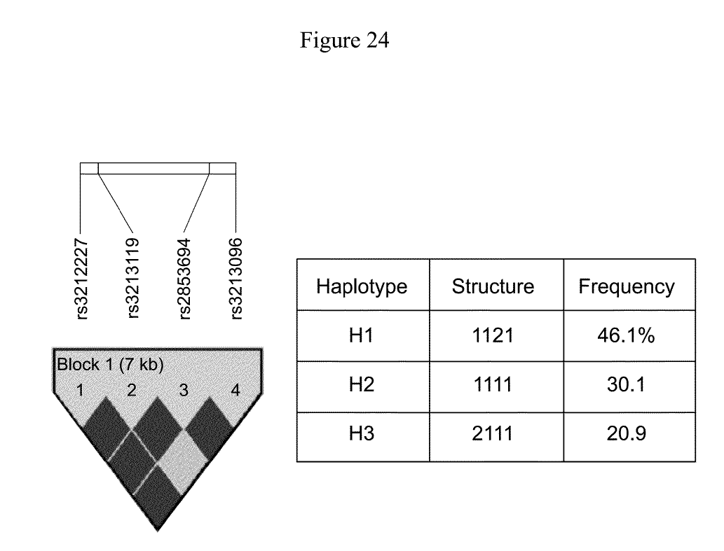

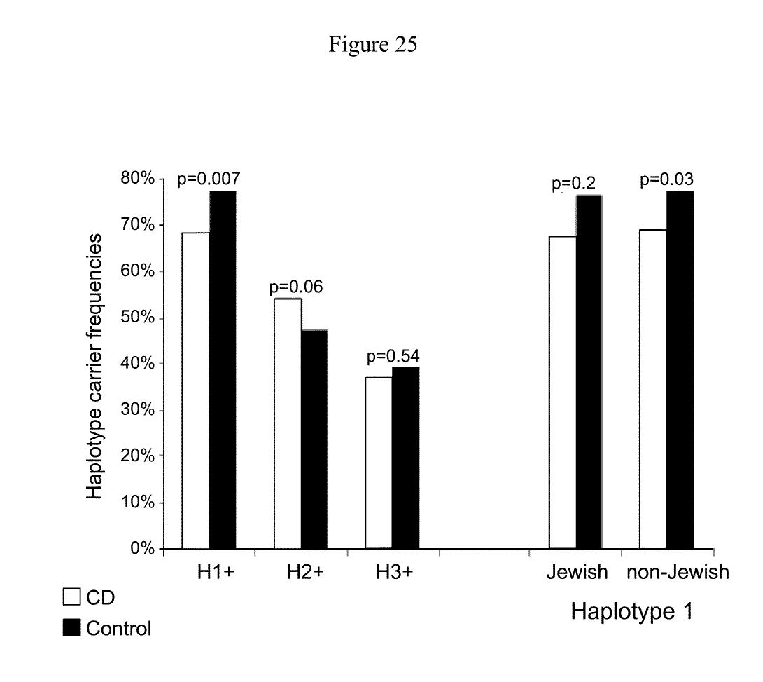

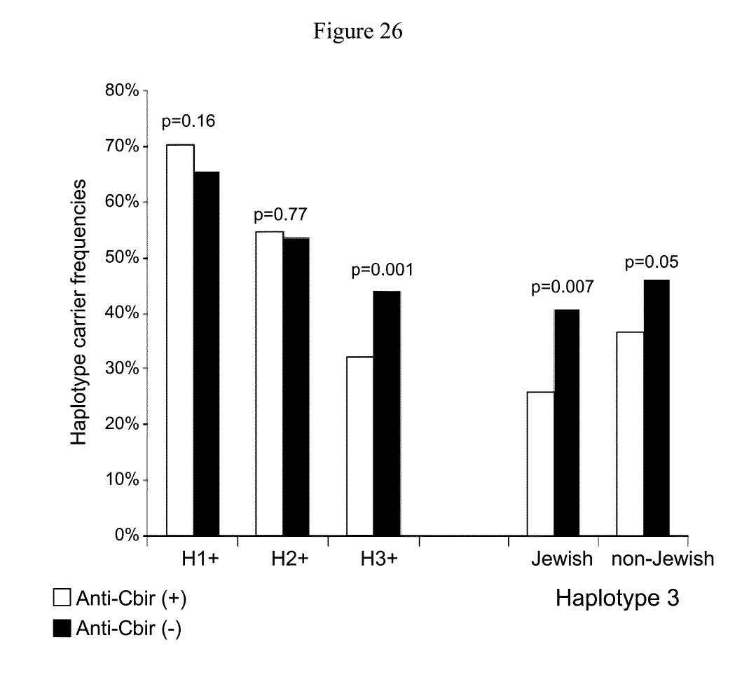

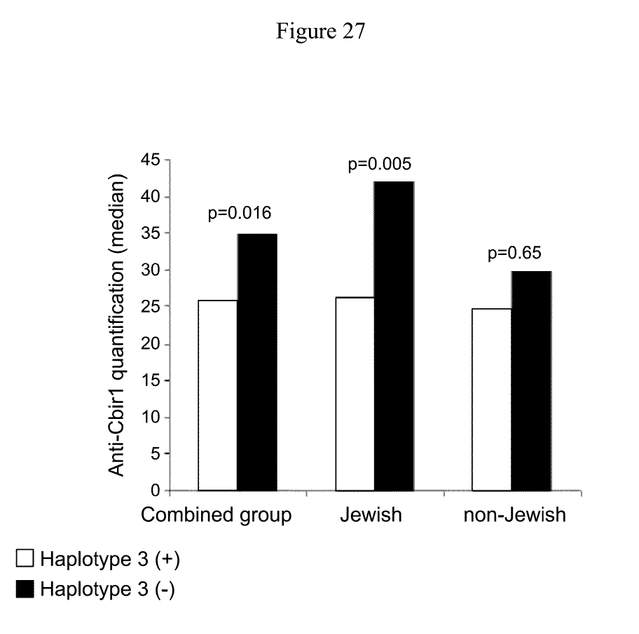

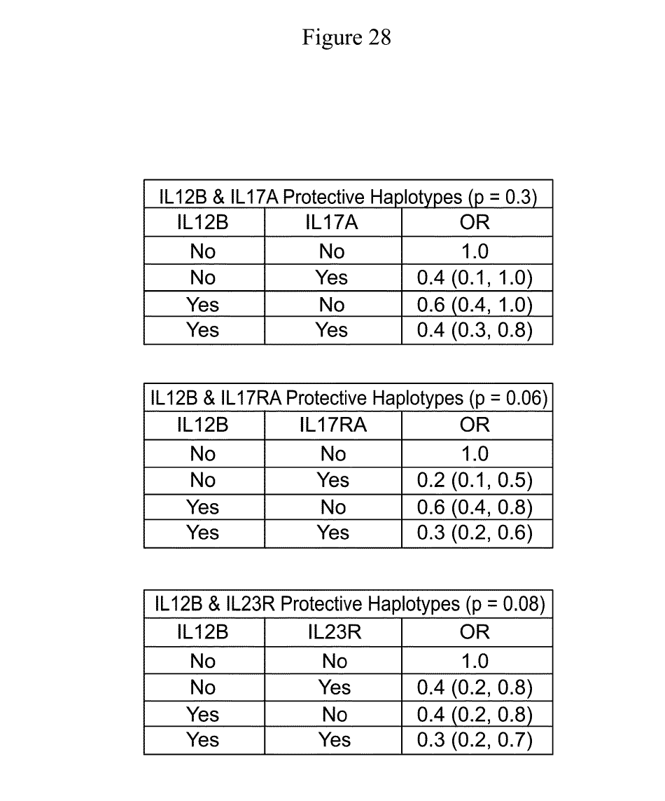

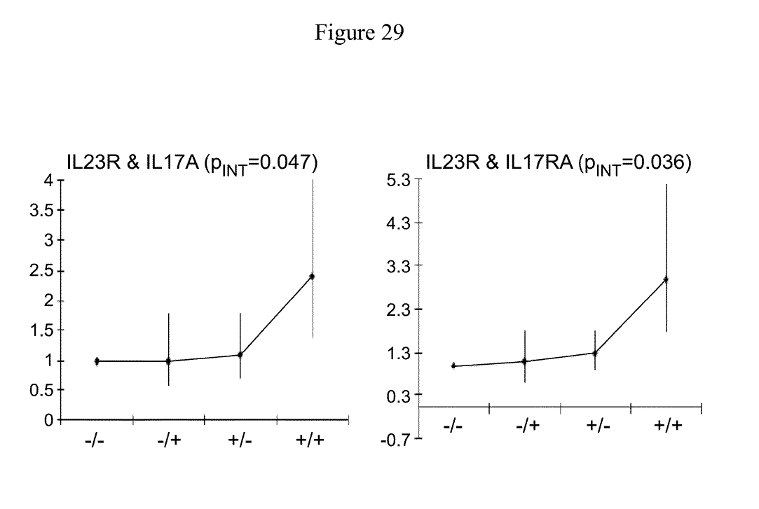

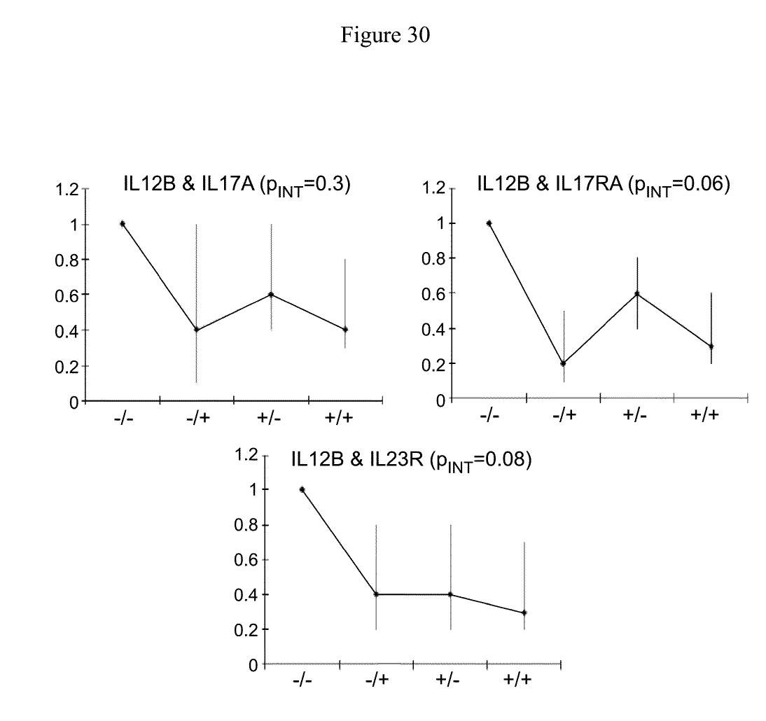

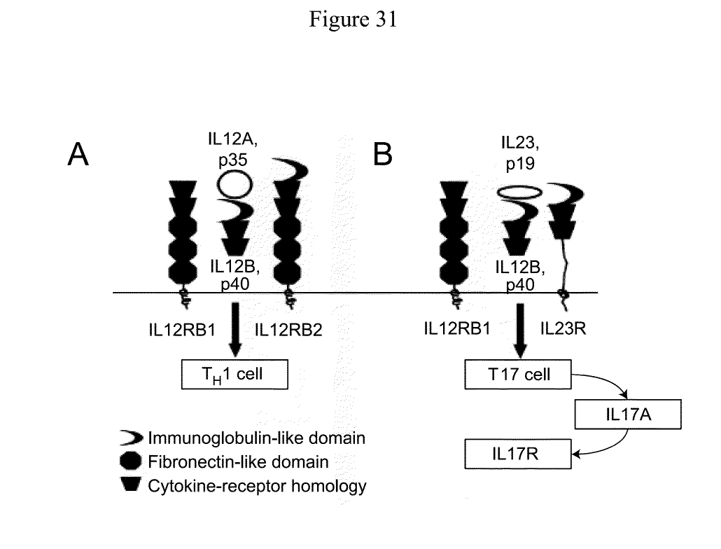

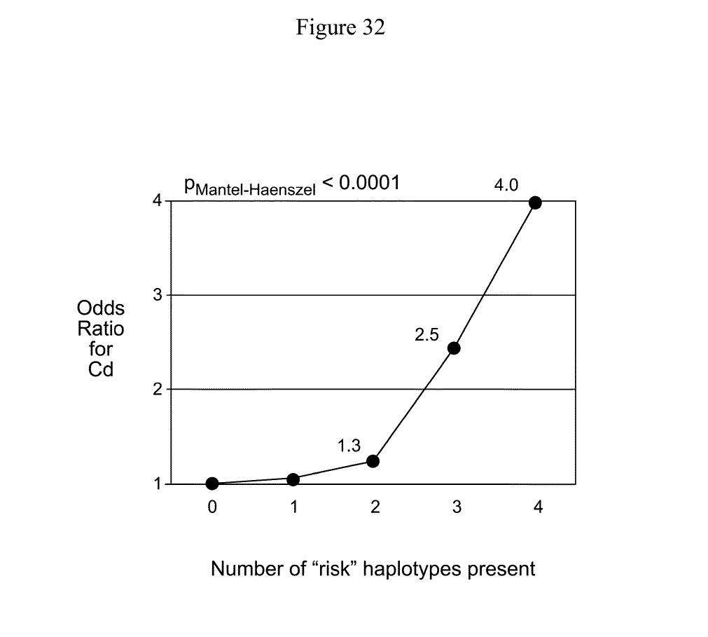

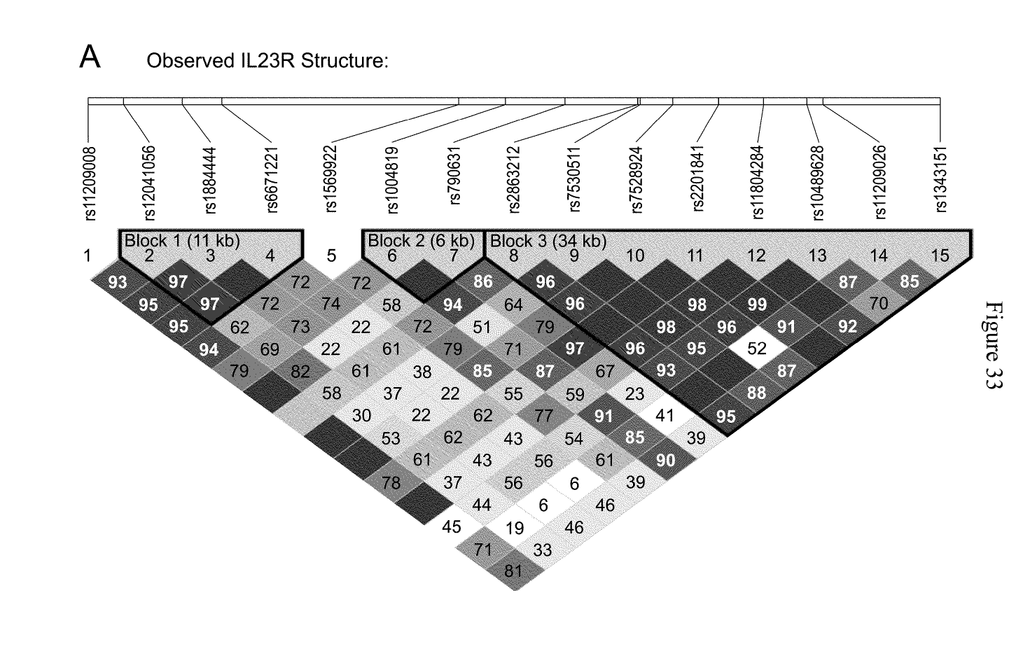

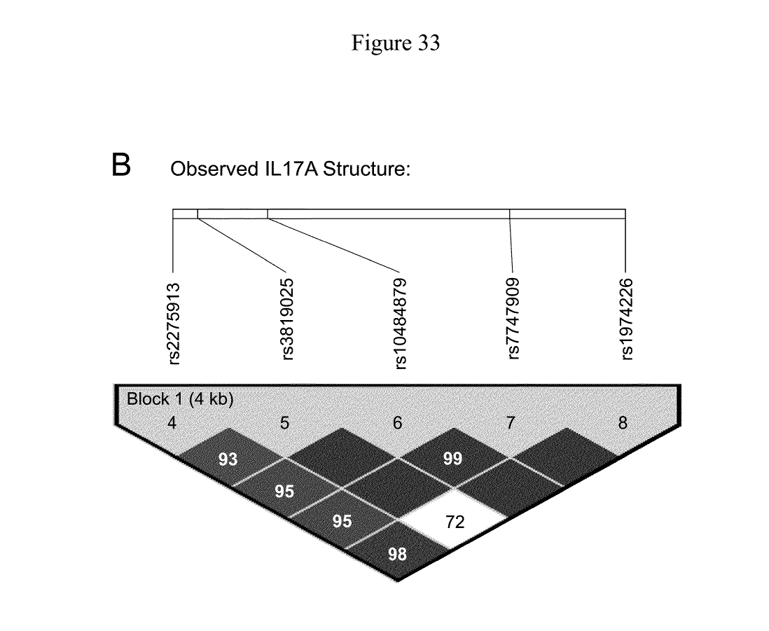

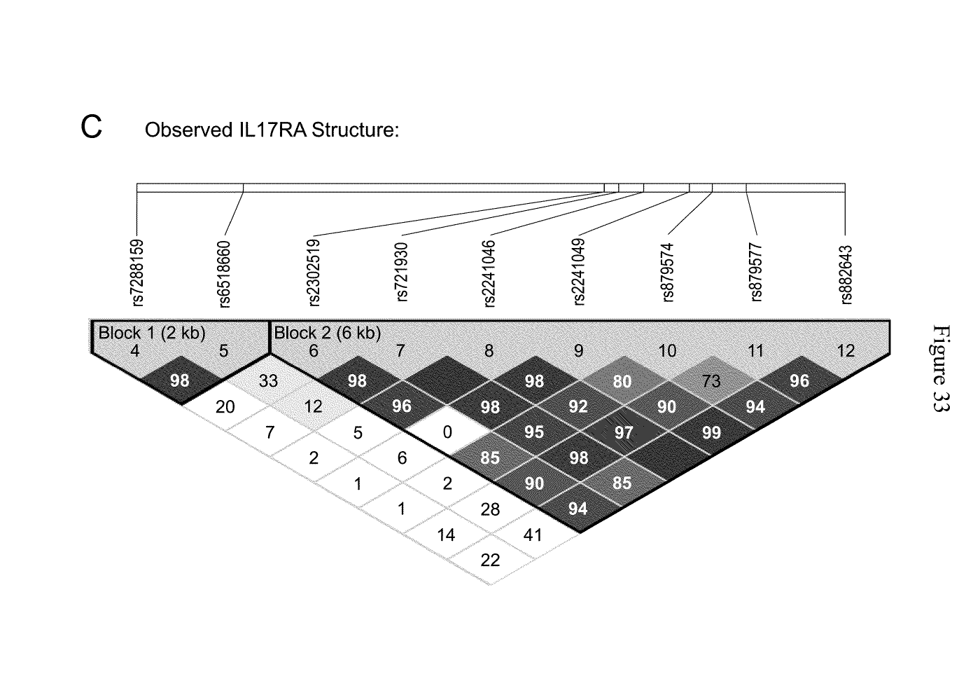

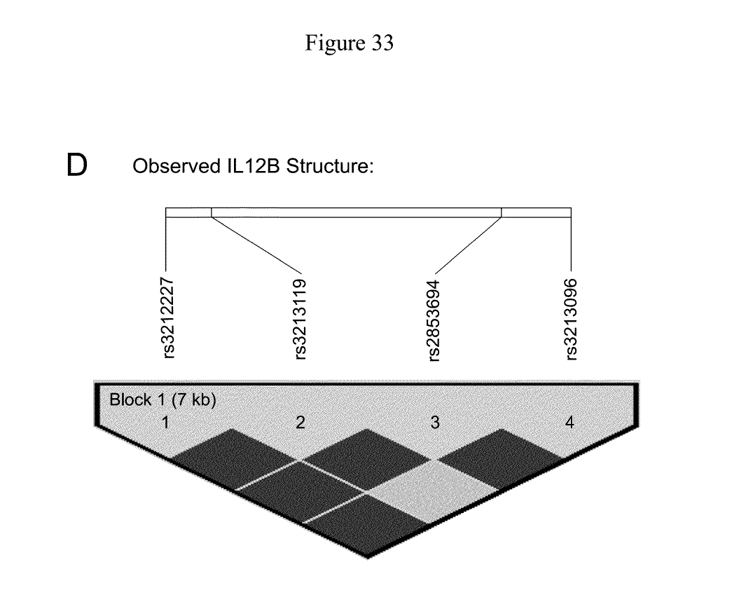

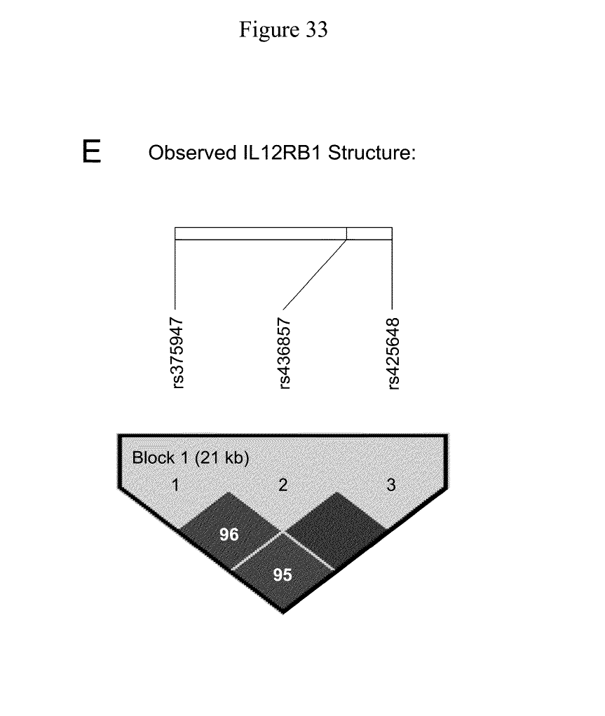

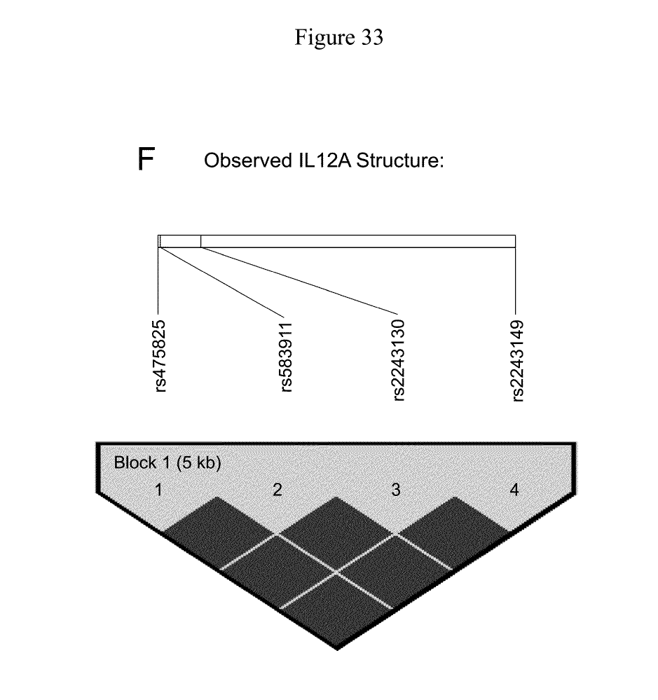

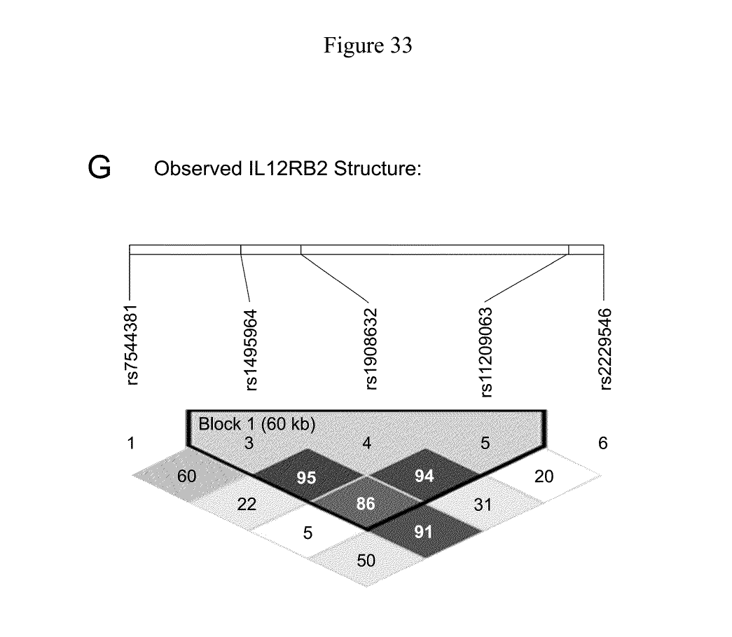

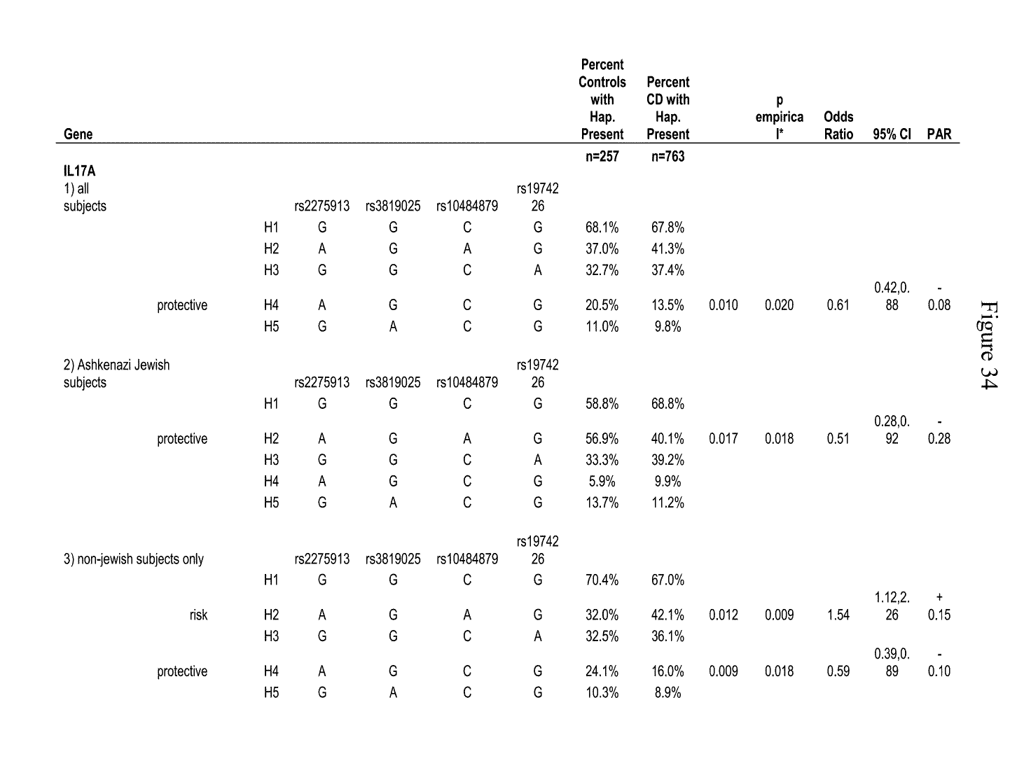

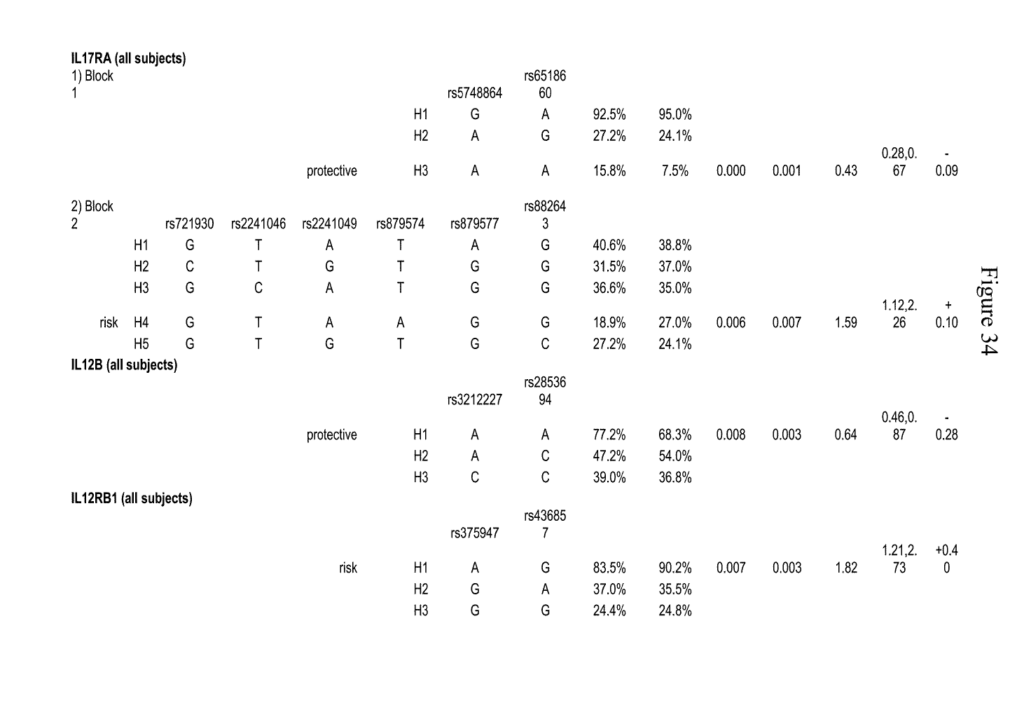

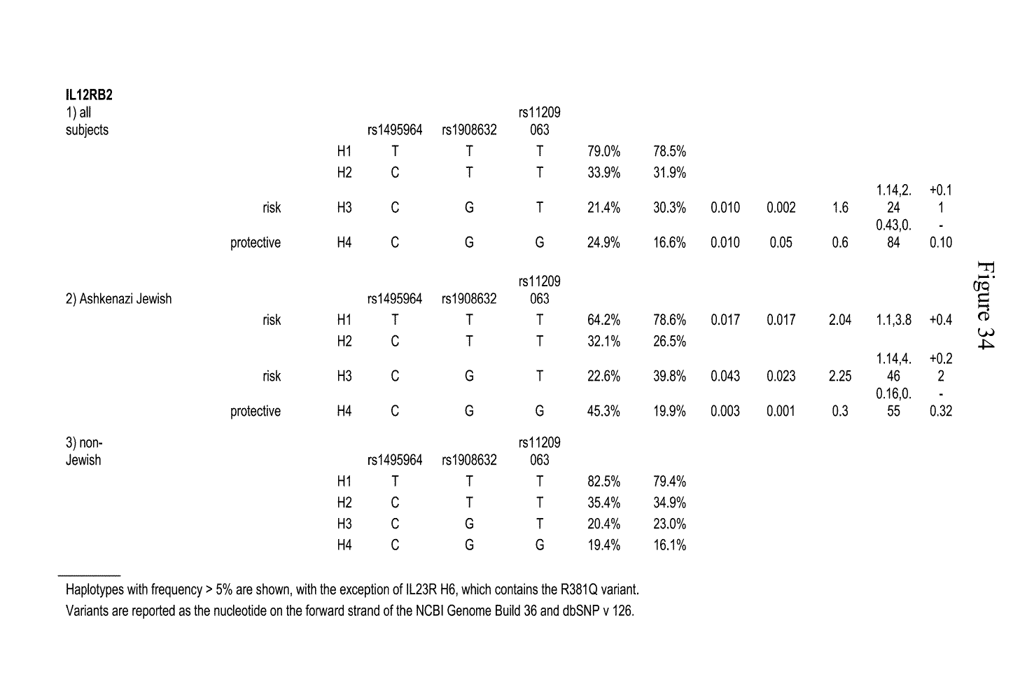

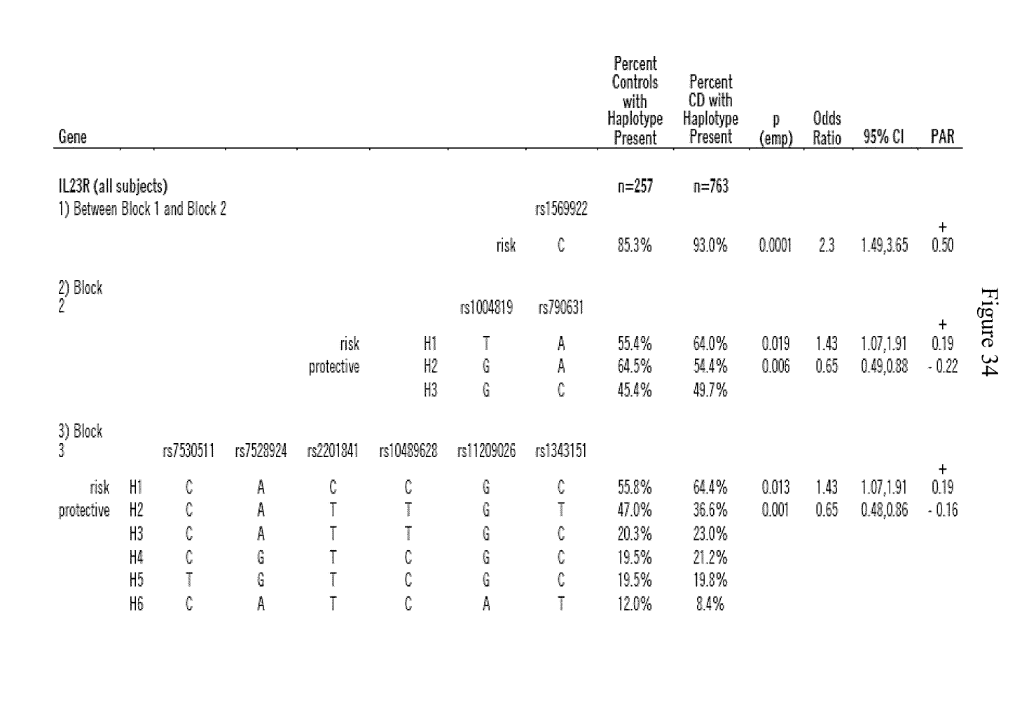

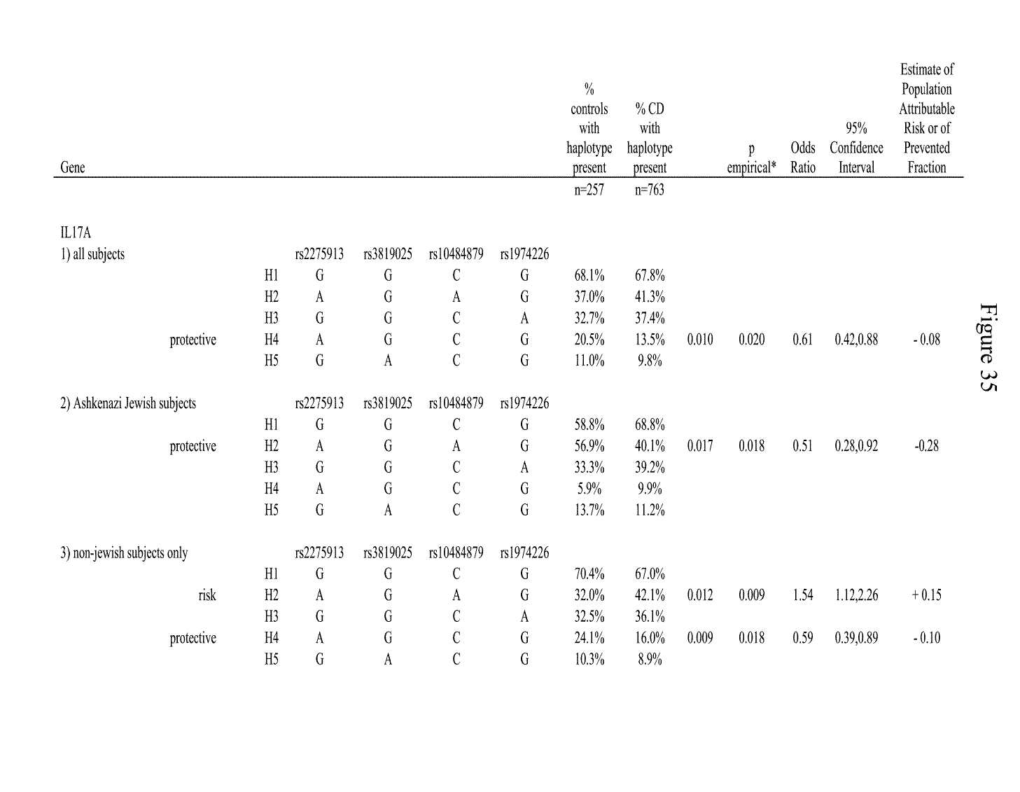

The identities of the IL12B markers, their location on the gene and their nucleotide substitutions may be found in FIG. 24.

As disclosed herein, an example of an IL23R genetic sequence is described as SEQ ID NO: 1. An example of an IL23R peptide sequence is described herein as SEQ ID NO: 2.

As disclosed herein, an example of an IL17A genetic sequence is described as SEQ ID NO: 3. An example of an IL17A peptide sequence is described herein as SEQ ID NO: 4.

As disclosed herein, an example of an IL17RA genetic sequence is described as SEQ ID NO: 5. An example of an IL17RA peptide sequence is described herein as SEQ ID NO: 6.

As disclosed herein, an example of an IL12B(p40) genetic sequence is described as SEQ ID NO: 7. An example of an IL12B(p40) peptide sequence is described herein as SEQ ID NO: 8.

As used herein, IL23R SNPs rs1004819 (SEQ ID NO: 9), rs790631 (SEQ ID NO: 10), rs2863212 (SEQ ID NO: 11), rs7530511 (SEQ ID NO: 12), rs7528924 (SEQ ID NO: 13), rs2201841 (SEQ ID NO: 14), rs11804284 (SEQ ID NO: 15), rs10489628 (SEQ ID NO: 16), rs11209026 (SEQ ID NO: 17), rs1343151 (SEQ ID NO: 18), rs1569922 (SEQ ID NO: 37) and rs11465804 (SEQ ID NO: 61). Examples of the IL23R genetic sequence are provided herein as SEQ ID NO: 1 and SEQ ID NO: 43.

As used herein, IL17A SNPs rs2275913 (SEQ ID NO: 1), rs3819025 (SEQ ID NO: 20), rs10484879 (SEQ ID NO: 21), rs7747909 (SEQ ID NO: 22) and rs1974226 (SEQ ID NO: 23). Examples of the IL17A genetic sequence are provided herein as SEQ ID NO: 3 and SEQ ID NO: 44.

As used herein, IL17RA SNPs rs7288159 (SEQ ID NO: 24), rs6518660 (SEQ ID NO: 25), rs2302519 (SEQ ID NO: 26), rs721930 (SEQ ID NO: 27), rs2241046 (SEQ ID NO: 28), rs2241049 (SEQ ID NO: 29), rs879574 (SEQ ID NO: 30), rs879577 (SEQ ID NO: 31) and rs882643 (SEQ ID NO: 32). Examples of the IL17RA genetic sequence are provided herein as SEQ ID NO: 5 and SEQ ID NO: 45.

Examples of the IL12B(p40) (IL12B) polymorphisms rs3212227 (SEQ ID NO: 33), rs3213119 (SEQ ID NO: 34), rs2853694 (SEQ ID NO: 35), and rs3213096 (SEQ ID NO: 36).

As used herein, IL12RB1 SNPs rs375947 (SEQ ID NO: 38), rs436857 (SEQ ID NO: 39) and rs425648 (SEQ ID NO: 62). Examples of the IL12RB1 genetic sequence are provided herein as SEQ ID NO: 46, SEQ ID NO: 47, SEQ ID NO: 48 and SEQ ID NO: 49.

As used herein, IL12RB2 SNPs rs1495964 (SEQ ID NO: 40), rs1908632 (SEQ ID NO: 41) and rs11209063 (SEQ ID NO: 42). Examples of the IL12RB2 genetic sequence are provided herein as SEQ ID NO: 50 and SEQ ID NO: 51.

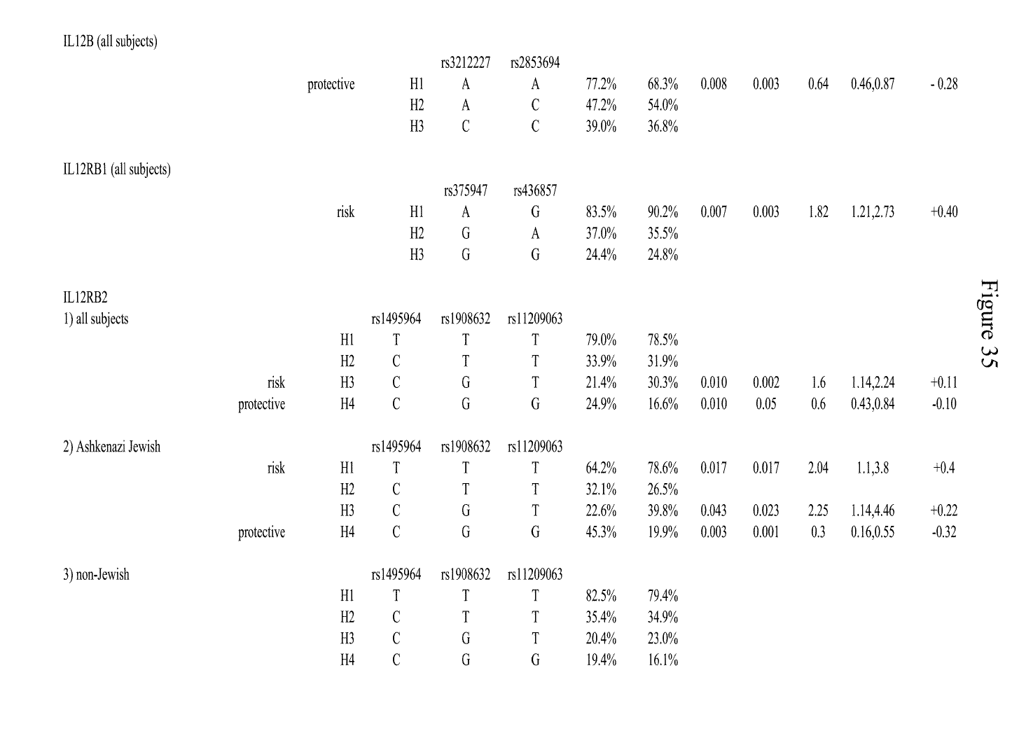

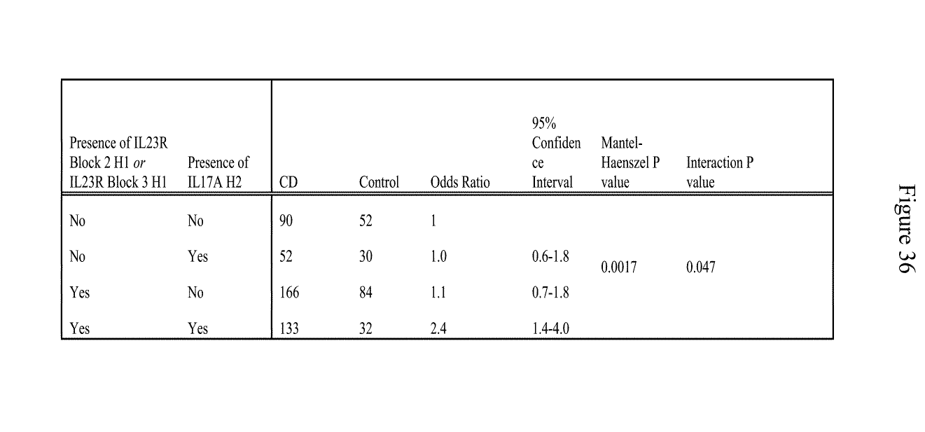

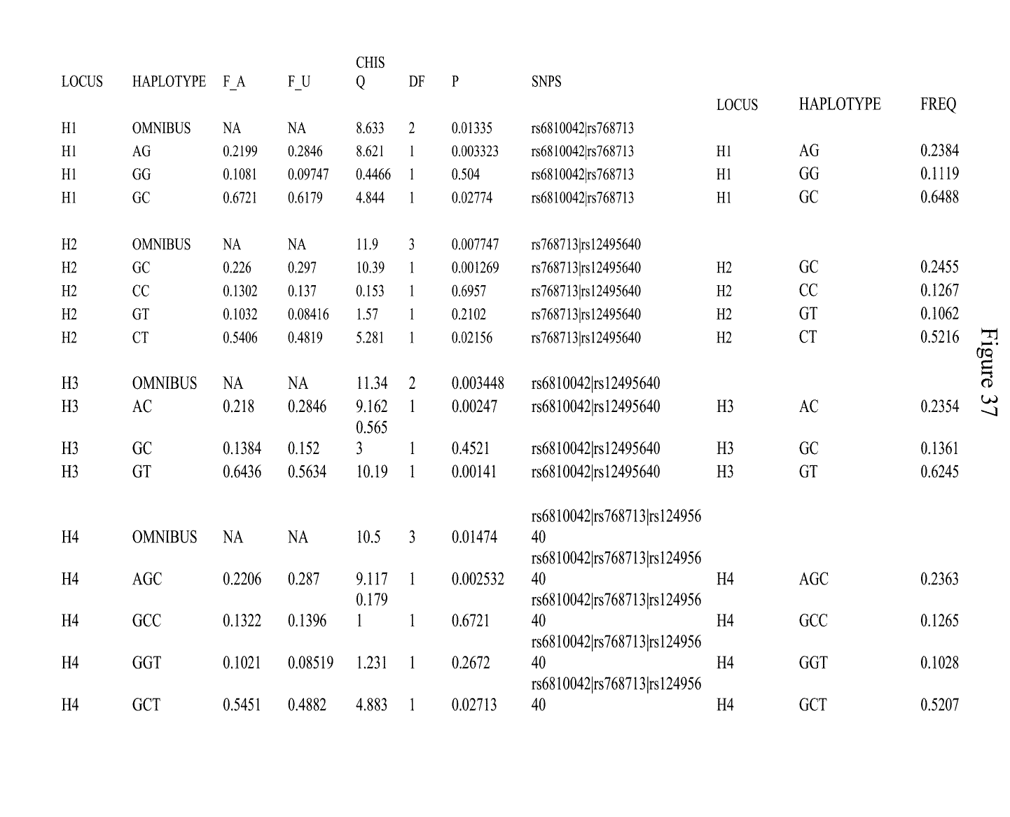

As used herein, the abbreviation “B” designates a haplotype block and “H” designates a haplotype. For example, “IL17RD B2H2” refers to Block 2 Haplotype 2 at the IL17RD genetic locus. Similarly, “IL17RD B1H2” and “IL17RD B2H3” refers to Block 1 Haplotype 2 and Block 2 Haplotype 3, respectively, at the IL17RD genetic locus. Although in no way limited, various SNPs and alleles described in FIG. 35 herein may be used to describe the various haplotypes referenced herein. For example, Block 2 at the IL17RD genetic locus includes SNPs rs12495640 (SEQ ID NO: 52), rs6788981 (SEQ ID NO: 53), and rs7374667 (SEQ ID NO: 54). Similarly, Block 1 at the IL17RD genetic locus includes SNPs rs6809523 (SEQ ID NO: 55), rs2129821 (SEQ ID NO: 56), rs17057718 (SEQ ID NO: 57), rs6780995 (SEQ ID NO: 58), rs747089 (SEQ ID NO: 59), and rs6810042 (SEQ ID NO: 60). Similarly, risk haplotype H4 at the IL12RB2 genetic locus includes SNPs rs1495964 (SEQ ID NO: 40), rs1908632 (SEQ ID NO: 41), and rs11209063 (SEQ ID NO: 42).

As known to one of ordinary skill in the art, there are presently various treatments and therapies available for those diagnosed with Inflammatory Bowel Disease, including but not limited to surgery, anti-inflammatory medications, steroids, and immunosuppressant's.

The inventors performed a genome-wide association study testing autosomal single nucleotide polymorphisms (SNPs) on the Illumina HumanHap300 Genotyping BeadChip. Based on these studies, the inventors found single nucleotide polymorphisms (SNPs) and haplotypes that are associated with increased or decreased risk for inflammatory bowel disease, including but not limited to CD. These SNPs and haplotypes are suitable for genetic testing to identify at risk individuals and those with increased risk for complications associated with serum expression of Anti-Saccharomyces cerevisiae antibody, and antibodies to I2, OmpC, and Cbir. The detection of protective and risk SNPs and/or haplotypes may be used to identify at risk individuals predict disease course and suggest the right therapy for individual patients. Additionally, the inventors have found both protective and risk allelic variants for Crohn's Disease and Ulcerative Colitis.

Based on these findings, embodiments of the present invention provide for methods of diagnosing and/or predicting susceptibility for or protection against inflammatory bowel disease including but not limited to Crohn's Disease and ulcerative colitis. Other embodiments provide for methods of prognosing inflammatory bowel disease including but not limited to Crohn's Disease and ulcerative colitis. Other embodiments provide for methods of treating inflammatory bowel disease including but not limited to Crohn's Disease and ulcerative colitis.

The methods may include the steps of obtaining a biological sample containing nucleic acid from the individual and determining the presence or absence of a SNP and/or a haplotype in the biological sample. The methods may further include correlating the presence or absence of the SNP and/or the haplotype to a genetic risk, a susceptibility for inflammatory bowel disease including but not limited to Crohn's Disease and ulcerative colitis, as described herein. The methods may also further include recording whether a genetic risk, susceptibility for inflammatory bowel disease including but not limited to Crohn's Disease and ulcerative colitis exists in the individual. The methods may also further include a prognosis of inflammatory bowel disease based upon the presence or absence of the SNP and/or haplotype. The methods may also further include a treatment of inflammatory bowel disease based upon the presence or absence of the SNP and/or haplotype.

In one embodiment, a method of the invention is practiced with whole blood, which can be obtained readily by non-invasive means and used to prepare genomic DNA, for example, for enzymatic amplification or automated sequencing. In another embodiment, a method of the invention is practiced with tissue obtained from an individual such as tissue obtained during surgery or biopsy procedures.

In one embodiment, the present invention provides methods of diagnosing and/or predicting susceptibility to Crohn's Disease in an individual by determining the presence or absence of IL23R risk haplotype H1 of Block 2. In another embodiment, the present invention provides methods of diagnosing and/or predicting protection against Crohn's Disease in an individual by determining the presence or absence of IL23R protective haplotype H2 in Block 2.

In another embodiment, the present invention provides methods of treatment of Crohn's Disease in an individual by determining the presence or absence of IL23R risk haplotype H1 of Block 2, and then treating the Crohn's Disease.

In one embodiment, the present invention provides methods of diagnosing and/or predicting susceptibility to Crohn's Disease in an individual by determining the presence or absence of IL23R risk haplotype H1 of Block 3. In another embodiment, the present invention provides methods of diagnosing and/or predicting protection against Crohn's Disease in an individual by determining the presence or absence of IL23R protective haplotype H2 in Block 3. In another embodiment, the present invention provides methods of diagnosing and/or predicting protection against Crohn's Disease in an individual by determining the presence or absence of IL23R protective haplotype H6 in Block 3.

In another embodiment, the present invention provides methods of treatment of Crohn's Disease in an individual by determining the presence or absence of IL23R risk haplotype H1 of Block 3, and then treating the Crohn's Disease.

In one embodiment, the present invention provides methods of diagnosing and/or predicting susceptibility to Crohn's Disease in an individual by determining the presence or absence of IL23R risk haplotype H1 of Block 2 and/or IL23R risk haplotype H1 of Block 3.

In another embodiment, the present invention provides methods of treatment of Crohn's Disease in an individual by determining the presence or absence of IL23R risk haplotype H1 of Block 2 and/or IL23R risk haplotype H1 of Block 3, followed by administering treatment of the Crohn's Disease.

In one embodiment, the present invention provides methods of diagnosing and/or predicting protection against Crohn's Disease in an individual by determining the presence or absence of IL23R protective haplotype H2 in Block 2, IL23R protective haplotype H2 in Block 3, and/or IL23R protective haplotype H6 in Block 3.

In one embodiment, the present invention provides methods of diagnosing and/or predicting susceptibility to Crohn's Disease in an individual by determining the presence or absence of IL23R risk haplotype H1 of Block 3 and increased serum expression of anti-I2 antibody.

In another embodiment, the present invention provides methods of treatment of Crohn's Disease in an individual by determining the presence or absence of IL23R risk haplotype H1 of Block 3 and increased serum expression of anti-I2 antibody, followed by administering treatment for the Crohn's Disease.

As further disclosed herein, IL23R risk haplotypes confer marked, additional CD risks compared with the functional, protective SNP IL23R R381Q. IL23R therefore accounts for a substantial increase in CD risk. Furthermore, IL23R haplotypes are associated with serum expression of antibody to 12, a Pseudomonas related antigen. Subjects with these haplotypes will be important for studying IL23R function.

In one embodiment, the present invention provides methods of diagnosing and/or predicting protection against IBD in an individual by determining the presence or absence of the protective R381Q SNP (rs11209026) of the IL23R gene. In another embodiment, the IBD comprises Crohn's Disease. In another embodiment, the IBD comprises ulcerative colitis. In another embodiment, the individual is a pediatric. In another embodiment, the individual is non-Jewish.

In one embodiment, the present invention provides methods of diagnosing and/or predicting susceptibility to Crohn's Disease in a non-Jewish individual by determining the presence or absence of a high frequency of IL17A haplotype H2 and a lower frequency of IL17A haplotype H4. In another embodiment, the present invention provides methods of diagnosing and/or predicting susceptibility to Crohn's Disease in a Jewish individual by determining the presence or absence of a low frequency of IL17A haplotype H2.

In another embodiment, the present invention provides methods of treatment for Crohn's Disease in a non-Jewish individual by determining the presence or absence of a high frequency of IL17A haplotype H2 and a lower frequency of IL17A haplotype H4, followed by administering treatment for the Crohn's Disease. In another embodiment, the present invention provides methods of treatment for Crohn's Disease in a Jewish individual by determining the presence or absence of a low frequency of IL17A haplotype H2, followed by administering treatment for the Crohn's Disease.

In one embodiment, the present invention provides methods of diagnosing and/or predicting susceptibility to Inflammatory Bowel Disease in an individual by determining the presence or absence of a low frequency of IL17RA haplotype H3 and a high frequency of IL17RA haplotype H4.

In another embodiment, the present invention provides methods of treatment for Inflammatory Bowel Disease in an individual by determining the presence or absence of a low frequency of IL17RA haplotype H3 and a high frequency of IL17RA haplotype H4, and then administering treatment for the Crohn's Disease.

In one embodiment, the present invention provides methods of diagnosing and/or predicting susceptibility for Crohn's Disease in an individual by determining the presence or absence of one or more risk haplotypes at the IL-23R locus and/or the IL-17A locus. In another embodiment, the present invention provides methods of treatment of Crohn's Disease in an individual by determining the presence or absence of one or more risk haplotypes as the IL23R locus and/or the IL-17A locus, and then administering a treatment for the Crohn's Disease.

In one embodiment, the present invention provides methods of diagnosing and/or predicting susceptibility to Crohn's Disease in an individual by determining the presence or absence of IL23R risk haplotype block 3 H1, IL23R risk haplotype block 2 H1, and/or IL17A risk haplotype H2. In another embodiment, the present invention provides methods of treatment of Crohn's Disease in an individual by determining the presence or absence of IL23R risk haplotype block 3 H1, IL23R risk haplotype block 2 H1, and/or I17A risk haplotype H2.

In one embodiment, the present invention provides methods of diagnosing and/or predicting susceptibility for Crohn's Disease in an individual by determining the presence or absence of one or more risk haplotypes at the IL-23R locus and/or IL-17RA locus. In another embodiment, the present invention provides methods of treatment of Crohn's Disease in an individual by determining the presence or absence of one or more risk haplotypes at the IL-23R locus and/or IL-17RA locus, and then administering a treatment for the Crohn's Disease.

In one embodiment, the present invention provides methods of diagnosing and/or predicting susceptibility to Crohn's Disease in an individual by determining the presence or absence of IL23R risk haplotype block 3 H1, IL23R risk haplotype block 2 H1, and/or IL17RA risk haplotype H4. In another embodiment, the present invention provides methods of treatment of Crohn's Disease in an individual by determining the presence or absence of IL23R risk haplotype block 3 H1, IL23R risk haplotype block 2 H1, and/or IL17RA risk haplotype H4, and then administering a treatment for the Crohn's Disease.

In one embodiment, the present invention provides methods of diagnosing and/or predicting protection against Crohn's Disease in an individual by determining the presence or absence of H1. In another embodiment, the present invention provides methods of treatment of Crohn's Disease in an individual by determining the presence or absence of H1, and then administering a treatment for the Crohn's Disease.

In one embodiment, the present invention provides methods of diagnosing and/or predicting protection against Crohn's Disease in an individual by determining the presence or absence of H3 with a lack of anti-Cbir1 expression. In another embodiment, the present invention provides methods of treatment of Crohn's Disease in an individual by determining the presence or absence of H3 with a lack of anti-Cbir1 expression, and then administering a treatment for the Crohn's Disease.

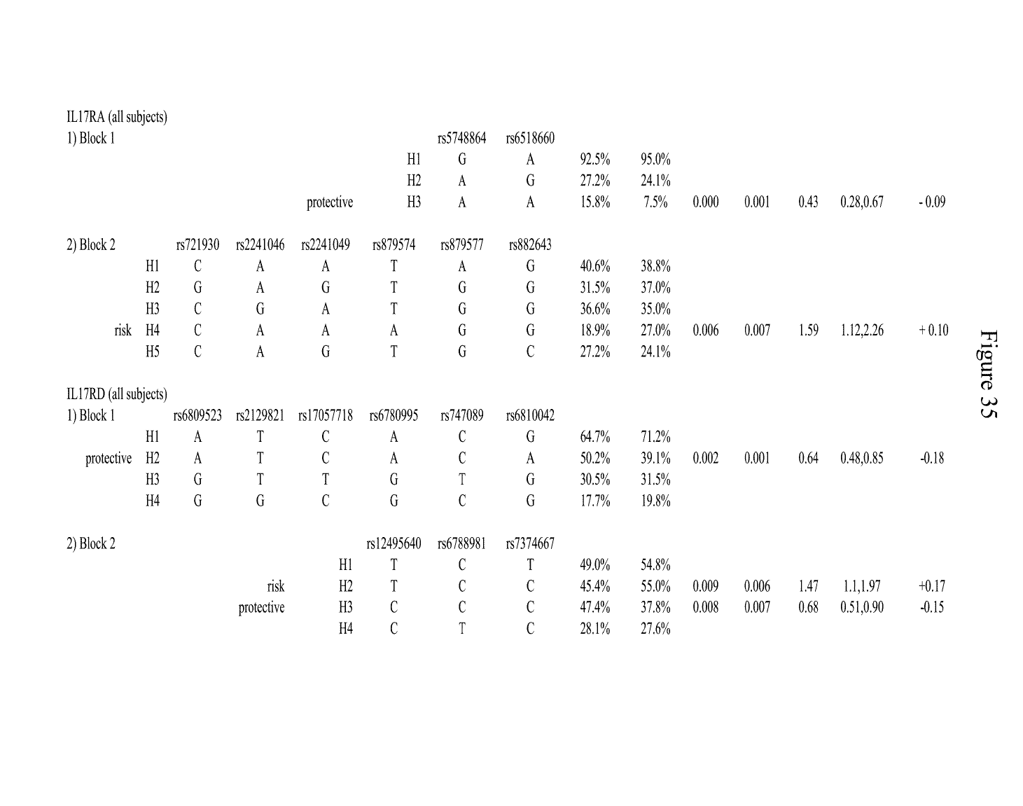

In one embodiment, a subject is at increased risk of IBD if the subject has the presence of B2H1, B3H1, or a combination thereof in IL23R, and absence of B2H2, B3H2 and B3H6 in I123R, as exemplified in FIG. 34, FIG. 35, Table A and Table B.

In one embodiment, a subject is at increased risk of IBD if the subject has the presence of H2 haplotype in IL17A and the absence of H4 haplotypes in IL17A, as exemplified in FIGS. 34 and 35.

In one embodiment, a subject is at increased risk of IBD if the subject has the presence of B2H4 in IL17RA and the absence of B1H3 in IL17RA, as exemplified in FIGS. 34 and 35.

In one embodiment, a subject is at increased risk of IBD if the subject has (i) the presence of B2H1, B3H1 or a combination thereof in IL23R, and absence of B2H2, B3H2, B3H6, or a combination thereof in I123R; (ii) the presence of H2 haplotype in IL17A and the absence of H4 haplotypes in IL17A; (iii) the presence of B2H4 in IL17RA and the absence of B1H3 in IL17RA, or a combination thereof.

In one embodiment, a subject is at decreased risk of IBD if the subject has the absence of the H1 haplotype in IL12B(p40) and/or the H3 haplotype in IL12B(p40) plus Cbir1 antibody expression, as exemplified in FIGS. 34 and 35.

In one embodiment, a non-Jewish subject is at an increased risk of IBD if the subject has the presence of any one or more of B2H1, B3H1 or a combination thereof in IL23R and absence of B2H2, B3H2 and B3H6 in IL23R. In another embodiment, the non-Jewish subject is a child.

In one embodiment, a non-Jewish subject is at an increased risk of IBD if the subject has (i) the presence of any one or more of B2H1, B3H1 or a combination thereof in IL23R and absence of B2H3, B3H2 and B3H6 in IL23R and (ii) presence of H2 haplotype in IL17A and absence of H4 haplotype in IL17A.

In one embodiment, a Jewish subject is at an increased risk of IBD if the subject has (i) the presence of any one or more of B2H1, B3H1 or a combination thereof in IL23R and absence of B2H2, B3H2 and B3H6 in IL23R; (ii) presence of H4 haplotype in IL17A and absence of H2 haplotype in IL17A.

TABLE A
IL23R Block 2 Haplotypes

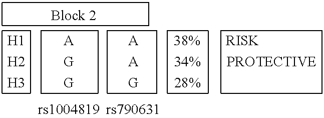

TABLE B
IL23R Block 3 Haplotypes

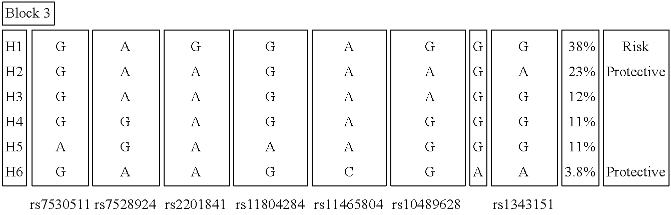

As disclosed herein, the inventors tested the hypothesis that risk haplotypes in genes of the IL23/IL17 pathway contribute to increased susceptibility for CD. 763 CD subjects and 254 controls were genotyped for single nucleotide polymorphisms in the IL23R, IL23R, IL17A, IL17RA, IL12A, IL12B, IL12RB1, and IL12RB2 genes. Genotyping was performed using both Illumina bead array and ABI TaqMan MGB technologies. Common haplotypes, with control frequencies greater than 5%, were assigned using Phase v2 and were tested for association with CD by chi square, with significance assessed using permutation.

As further disclosed herein, the inventors found that haplotypes with increased risk for CD were observed in the IL23R, IL17A, IL17RA genes, and IL12RB1 genes (IL23R, 55% control, 64% CD, p=0.015; IL17A, 32% control, 36% CD, p=0.015; IL17A, 19% control, 27% CD, p=0.003; IL12RB1, 84% control, 90% CD, p=0.004). These haplotypes substantially increase CD risk as seen by a large estimated population attributable risk (PAR, IL23R risk, ˜19%; IL17A risk, ˜16%; IL17RA risk, ˜10%). The odds ratio for CD increased with the number of risk haplotypes from these 4 genes (OR=1 for 0 or 1 risk haplotype, 1.3 for 2, 2.5 for 3, and 4 for 4 risk haplotypes, p<0.0001). Furthermore, a synergy was observed between IL23R and IL17A, and between IL23R and IL17RA, in that an increased odds ratio (OR) for CD was observed when a risk haplotype from both genes was present (OR ˜1 for the presence of the risk haplotype from IL23R or IL17A and 2.4 for both, p=0.047 for interaction; OR ˜1.1 for IL23R or IL17RA and ˜3 for both, p=0.036 for interaction). Similarly, no interaction between any of the genes tested and NOD2/CARD15 mutations was observed.

In one embodiment, the present invention provides methods of diagnosing and/or predicting susceptibility to Crohn's Disease by determining the presence or absence of risk haplotypes in IL23R, IL17A, and/or IL17RA genes. In another embodiment, the present invention provides methods of prognosis of Crohn's Disease by determining the presence or absence of risk haplotypes in IL23R, IL17A, and/or IL17RA genes. In another embodiment, the present invention provides methods of treatment of Crohn's Disease by inhibiting the IL23/IL17 pathway.

As disclosed herein, for IL12B, the tagSNPs formed one haplotype block and H1 was associated with a modestly decreased susceptibility for CD (“protective,” Controls, 77.2%, CD 68.3%, p=0.004) and a population attributable risk of minus ˜28%. For IL12RB1, the tagSNPs formed one haplotype block and H1 was associated with a greater susceptibility for CD (“risk,” Control, 83.5%, CD, 90.2%, p=0.004). For IL12RB2, the tagSNPs formed one haplotype block and H4 was associated with a decrease in susceptibility for CD (“protective,” Control, 24.3%, CD, 18.5%, p=0.036). In contrast to the other observed associations, this association of CD and IL12RB2 haplotypes was particular to Ashkenazi Jewish subjects because when Ashkenazi Jewish and non-Jewish CD subjects were analyzed separately, the association of CD and the IL12RB2 H4 protective haplotype was observed in the Jewish subjects only (Jewish: Control. 43.4%, CD 21.9%, p=0.001; non-Jewish: Control, 19.4%, CD, 16.1%, p is not significant). Furthermore, a significant risk haplotype for this population was also observed, the presence of IL12RB2 H1 (Jewish: Control, 62.3%, CD, 78.6%, p=0.009; non-Jewish: Control, 82.5%, CD, 79.4%, p not significant).

In one embodiment, the present invention provides methods of diagnosing and/or predicting susceptibility to Crohn's Disease in an individual by determining the presence or absence of H1 susceptibility haplotype of IL12RB1 in the individual. In another embodiment, the present invention provides method of treatment of Crohn's Disease in an individual by inhibiting the expression of H1 susceptibility haplotype of IL12RB1 in the individual. In another embodiment, the present invention provides method of treatment of Crohn's Disease by determining the presence or absence of H1 susceptibility haplotype of IL12RB1 and treating the Crohn's Disease.

In one embodiment, the present invention provides methods of diagnosing and/or predicting susceptibility to Crohn's Disease in a Jewish individual by determining the presence or absence of H1 susceptibility haplotype of IL12RB2 in the Jewish individual. In another embodiment, the present invention provides methods of treatment of Crohn's Disease by inhibiting the expression of H1 susceptibility haplotype of IL12RB2 in the Jewish individual. In another embodiment, the present invention provides method of treatment of Crohn's Disease by determining the presence of H1 susceptibility haplotype of IL12RB2 in the Jewish individual and treating the Crohn's Disease.

In one embodiment, the present invention provides methods of diagnosing and/or predicting susceptibility to Crohn's Disease in an individual by determining the presence or absence of H3 susceptibility haplotype of IL12RB2 in the individual. In another embodiment, the present invention provides methods of treatment of Crohn's Disease by inhibiting the expression of H3 susceptibility haplotype of IL12RB2 in the individual. In another embodiment, the present invention provides method of treatment of Crohn's Disease by determining the presence of H3 susceptibility haplotype of IL12RB2 in the individual and treating the Crohn's Disease.

In one embodiment, the present invention provides methods of diagnosing and/or predicting protection against Crohn's Disease in a Jewish individual by determining the presence or absence of H4 protective haplotype of IL12RB2 in the individual. In another embodiment, the present invention provides methods of prognosis of Crohn's Disease in an individual by determining the presence or absence of H4 protective haplotype of IL12RB2 in the individual. In another embodiment, the present invention provides methods of treatment of Crohn's Disease in an individual by inhibiting the expression of H4 protective haplotype of IL12RB2 in the individual.

In one embodiment, a subject is at increased risk of IBD if the subject has the presence of B2H1, B3H1 or a combination thereof in IL23R, and absence of B2H2, B3H2 and B3H6 in I123R, as exemplified in FIG. 34, FIG. 35, Table A and Table B.

In one embodiment, a subject is at increased risk of IBD if the subject has the presence of H2 haplotype in IL17A and the absence of H4 haplotypes in IL17A, as exemplified in FIGS. 34 and 35.

In one embodiment, a subject is at increased risk of IBD if the subject has the presence of B2H4 in IL17RA and the absence of B1H3 in IL17RA, as exemplified in FIGS. 34 and 35.

In one embodiment, a subject is at increased risk of IBD if the subject has the presence B1H1 in IL12RB1, as exemplified in FIGS. 34 and 35.

In one embodiment, a subject is at increased risk of IBD if the subject has (i) the presence of B2H1, B3H1, or a combination thereof in IL23R, and absence of B2H2, B3H2 and B3H6, or a combination thereof in I123R; (ii) the presence of H2 haplotype in IL17A and the absence of H4 haplotypes in IL17A; (iii) the presence of B2H4 in IL17RA and the absence of B1H3 in IL17RA, (iv) the presence B1H1 in IL12RB1, a combination thereof.

In one embodiment, a subject of Ashkenazi Jewish decent is at an increased risk of IBD if the subject has the presence of the H1 and H3 haplotypes in IL12RB2 and the absence of B2H4, as exemplified in FIGS. 34 and 35.

As disclosed herein, the inventors have determined that IL17RD is associated with CD and that there is a gene-gene interaction within IL23-IL17 pathway genes. 763 CD subjects and 254 controls were genotyped for single nucleotide polymorphisms in the IL23R, IL23R, IL17A, IL17RA, IL12B, IL12RB1, IL12RB2 and IL17RD genes using Illumina and ABI platforms. Haplotypes were assigned using Phase v2 and were tested for association with CD by chi square test. The inventors utilized multidimensionality reduction (MDR) to explore gene-gene interactions.

As further disclosed herein, two Blocks (B) of IL17RD were associated with CD. CD patients had a higher frequency of haplotype2 in block2 (B2H2, 55.0% vs. 45.4%, OR=1.5, p=0.01) and a lower frequency of B1H2 (39.1% vs. 50.2%, OR=0.64, p=0.002) and B2H3 (37.8% vs. 47.4%, OR=0.68, p=0.01) when compared with controls. Haplotypes with increased risk for CD were observed in the IL23R_B2H1 and B3H1, IL17A_H2, IL17RA_B2H4, IL12RB1_H1 and IL12RB2_H3; haplotypes with decreased risk were observed in the IL23R_B2H2 and B3H2, IL17A_H4, IL17RA_B1H3, IL12B_H1 and IL12RB2_H4. MDR analysis suggested interaction between IL23R_B2H2, IL12RB2_H4 and IL17RD_B2H2 (CV consistency 10/10, tested accuracy 59.7%, p=0.002). The following logistic regression analysis confirmed the interaction (IL23R_B2H2*IL12RB2_H4, p<0.0001; IL23R_B2H2*IL17RD_B2H2, p=0.02). Thus, the inventors have found IL17RD to be significantly associated with CD and likely to interact with IL23R in the risk of developing CD.

In one embodiment, the present invention provides a method of diagnosing and/or predicting susceptibility to Crohn's Disease by determining the presence or absence of a risk haplotype and/or variant at the IL17RD locus, where the presence of the risk haplotype and/or variant at the IL17RD locus is indicative of susceptibility to Crohn's Disease. In another embodiment, the present invention provides a method of treating Crohn's Disease by determining the presence of a risk haplotype and/or variant at the IL17RD locus and treating the Crohn's Disease. In another embodiment, the risk haplotype at the IL17RD locus is IL17RD Block 2 Haplotype 2. In another embodiment, the present invention provides a method of diagnosing and/or predicting susceptibility to Crohn's Disease by determining the presence or absence of an interaction between IL17RD Block 2 Haplotype 2 and IL23R Block 2 Haplotype 2 and/or IL12RB2 Haplotype 4, where the presence of an interaction between IL17RD Block 2 Haplotype 2 and IL23R Block 2 Haplotype 2 and/or IL12RB2 Haplotype 4 is indicative of susceptibility to Crohn's Disease.

In another embodiment, the present invention provides a method of diagnosing and/or predicting protection against Crohn's Disease by determining the presence or absence of a protective haplotype at the IL17RD locus, where the presence of the protective haplotype at the IL17RD locus is indicative of a decreased likelihood of susceptibility to Crohn's Disease relative to a healthy individual. In another embodiment, the present invention provides a method of diagnosing and/or predicting protection against Crohn's Disease by determining the presence or absence of a protective variant at the IL17RD locus, where the presence of the protective variant at the IL17RD locus is indicative of a decreased likelihood of susceptibility to Crohn's Disease relative to a healthy individual. In another embodiment, the protective haplotype at the IL17RD locus is IL17RD Block 1 Haplotype 2. In another embodiment, the protective haplotype at the IL17RD locus is IL17RD Block 2 Haplotype 3.

In one embodiment, a subject is at increased risk of IBD if the subject has the presence of B2H1, B3H1 or a combination thereof in IL23R, and absence of B2H2, B3H2 and B3H6 in I123R, as exemplified in FIG. 34, FIG. 35, Table A and Table B.

In one embodiment, a subject is at increased risk of IBD if the subject has the presence of H2 haplotype in IL17A and the absence of H4 haplotypes in IL17A, as exemplified in FIGS. 34 and 35.

In one embodiment, a subject is at increased risk of IBD if the subject has the presence of B2H4 in IL17RA and the absence of B1H3 in IL17RA, as exemplified in FIGS. 34 and 35.

In one embodiment, a subject is at increased risk of IBD if the subject has the presence B1H1 in IL12RB1, as exemplified in FIGS. 34 and 35.

In one embodiment, a subject is at increased risk of IBD if the subject has the presence of B2H2 in IL17RD and the absence of B1H2 and B2H3 in IL17RD, as exemplified in FIG. 35.

In one embodiment, a subject is at increased risk of IBD if the subject has (i) the presence of B2H1, B3H1, or a combination thereof in IL23R, and absence of B2H2, B3H2 and B3H6, or a combination thereof in I123R; (ii) the presence of H2 haplotype in IL17A and the absence of H4 haplotypes in IL17A; (iii) the presence of B2H4 in IL17RA and the absence of B1H3 in IL17RA, (iv) the presence B1H1 in IL12RB1; (v) the presence of B2H2 in IL17RD and the absence of B1H2 and B2H3 in IL17RD and/or a combination thereof.

In one embodiment, a subject is at increased risk of IBD if the subject has (i) the presence of B2H1 and/or B3H1 in IL23R, or a combination thereof and absence of B2H2, B3H2 and B3H6 in IL23R, or a combination thereof; (ii) the presence of H2 haplotype in IL17A and the absence of H4 haplotypes in IL17A; (iii) the presence of B2H4 in IL17RA and the absence of B1H3 in IL17RA; (iv) the absence of H1 haplotype in IL12B(p40) and the H3 haplotype in IL12B(p40) plus Cbir1 antibody expression, (v) the presence B1H1 in IL and (v) the presence of B2H2 in IL17RD and the absence of B1H2 and B2H3 in IL17RD, and/or a combination thereof.

In another embodiment, the subject is at decreased risk of IBD if the subject has (i) the absence of B2H1 and/or B3H1 in IL23R, or a combination thereof and presence of B2H2, B3H2 and B3H6, or a combination thereof; (ii) the absence of H2 haplotype in IL17A and the presence of H4 haplotypes in IL17A; (iii) the absence of B2H4 in IL17RA and the presence of B1H3 in IL17RA; (iv) the presence of H1 haplotype in IL12B(p40) and the H3 haplotype in IL12B(p40) plus Cbir1 antibody expression, (v) the absence B1H1 in IL12RB1 and (v) the absence of B2H2 in IL17RD and the presence of B1H2 and B2H3 in IL17RD, and/or a combination thereof.

Various embodiments of the present invention provides for methods of diagnosing susceptibility to Inflammatory Bowel Disease (IBD) in a subject in need thereof, comprising: providing a sample from the subject; assaying the sample to detect risk and/or protective haplotypes in one or more genes selected from the group consisting of: IL23R, IL17A, IL17RA, IL17RD, IL12B(p40), IL12RB1 and IL12RB2; and determining that the subject has an increased susceptibility to IBD if one or more risk haplotypes are present and the protective haplotypes are absent or determining that the subject has a decreased susceptibility to IBD if one or more protective haplotypes are present and the risk haplotypes are absent. In other embodiment, IBD comprises Crohn's Disease (CD) and ulcerative colitis (UC). In another embodiment, the risk haplotypes located at the: IL23R locus are IL23R Block 2 Haplotype 1 (B2H1) and/or IL23R Block 3 Haplotype 1 (B3H1); IL17A locus are IL17A Haplotype 2 (H2); IL17RA locus is IL17RA Block 2 Haplotype 4 (B2H4); IL17RD locus is IL17RD Block 2 Haplotype 2 (B2H2); IL12RB1 locus is IL12RB1 Haplotype 1 (H1); and IL12RB2 locus are IL12RB2 Haplotype 1 (H1) and/or IL12RB2 Haplotype 3 (H3). In other embodiment, the protective haplotypes located at the: IL23R locus are IL23R Block 2 Haplotype 2 (B2H2), IL23R Block 3 Haplotype 2 (B3H2) and/or IL23R Block 3 Haplotype 6 (B3H6); IL17A locus is IL17A Haplotype 4 (H4); IL17RA locus is IL17RA Block 1 Haplotype 3 (B1H3); IL17RD locus are IL17RD Block 1 Haplotype 2 (B1H2) and/or IL17RD Block 2 Haplotype 3 (B2H3); IL12B(p40) locus are IL12B(p40) Haplotype 1 (H1) and/or IL12B(p40) Haplotype 3 (H3); and IL12RB2 locus is IL12RB2 Haplotype 4 (H4).

In various other embodiments, the detection of IL23R B2H1 and/or IL23R B3H1 in a pediatric non-Jewish subject indicates an increased susceptibility to IBD. In other embodiments, the detection of IL12RB2 H4 haplotype in a subject of Ashkenazi Jewish descent indicates a decreased susceptibility to IBD and wherein the detection of IL12B2 H1 and/or IL12B2H3 haplotypes in a subject of Ashkenazi Jewish descent indicates an increased susceptibility to IBD. In other embodiments, the detection of IL17A H2 haplotype in a non-Jewish subject or the detection of IL17A H4 haplotype in a Jewish subject indicates an increased susceptibility to IBD. In other embodiments, the detection of IL12B(p40) H3 haplotype and Cbir1 antibody expression indicates a decreased susceptibility to UC in a subject.

In various other embodiments, the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP) and denaturing gradient gel electrophoresis (DGGE). In other embodiments, the detection of risk and/or protective haplotypes is relative to that detected in a healthy subject. In other embodiments, the presence of ten risk haplotypes presents a greater susceptibility than the presence of nine, eight, seven, six, five, four, three, two, one or none of the risk haplotypes, and the presence of nine risk haplotypes presents a greater susceptibility than the presence of eight, seven, six, five, four, three, two, one or none of the risk haplotypes, and the presence of eight risk haplotypes presents a greater susceptibility than the presence of seven, six, five, four, three, two, one or none of the risk haplotypes, and the presence of seven risk haplotypes presents a greater susceptibility than the presence of six, five, four, three, two, one or none of the risk haplotypes, and the presence of six risk haplotypes presents a greater susceptibility than the presence of five, four, three, two, one or none of the risk haplotypes, and the presence of five risk haplotypes presents a greater susceptibility than the presence of four, three, two, one or none of the risk haplotypes, and the presence of four risk haplotypes presents a greater susceptibility than the presence of three, two, one or none of the risk haplotypes, and the presence of three risk haplotypes presents a greater susceptibility than the presence of two, one or none of the risk haplotypes, and the presence of two risk haplotypes presents a greater susceptibility than the presence of one or none of the risk haplotypes, and the presence of one risk haplotype presents a greater susceptibility than the presence of none of the risk haplotypes.

The invention also provides embodiments, for a process for selecting a therapy for a subject susceptible to IBD comprising providing a sample from the subject; assaying the sample to detect risk and/or protective haplotypes from genes selected from the group consisting of: IL23R, IL17A, IL17RA, IL17RD, IL12B(p40), IL12RB1 and IL12RB2; determining that the subject has an increased susceptibility to IBD if one or more risk haplotypes are present and the protective haplotypes are absent or determining that the subject has a decreased susceptibility to IBD if one or more protective haplotypes are present and the risk haplotypes are absent; and selecting a preventative therapy for a subject with an increased susceptibility to IBD. In other embodiments, IBD comprises Crohn's Disease (CD) and ulcerative colitis (UC). In another embodiment, the risk haplotypes located at the: IL23R locus are IL23R Block 2 Haplotype 1 (B2H1) and/or IL23R Block 3 Haplotype 1 (B3H1); IL17A locus are IL17A Haplotype 2 (H2); IL17RA locus is IL17RA Block 2 Haplotype 4 (B2H4); IL17RD locus is IL17RD Block 2 Haplotype 2 (B2H2); IL12RB1 locus is IL12RB1 Haplotype 1 (H1); and IL12RB2 locus are IL12RB2 Haplotype 1 (H1) and/or IL12RB2 Haplotype 3 (H3). In other embodiment, the protective haplotypes located at the: IL23R locus are IL23R Block 2 Haplotype 2 (B2H2), IL23R Block 3 Haplotype 2 (B3H2) and/or IL23R Block 3 Haplotype 6 (B3H6); IL17A locus is IL17A Haplotype 4 (H4); IL17RA locus is IL17RA Block 1 Haplotype 3 (B1H3); IL17RD locus are IL17RD Block 1 Haplotype 2 (B1H2) and/or IL17RD Block 2 Haplotype 3 (B2H3); IL12B(p40) locus are IL12B(p40) Haplotype 1 (H1) and/or IL12B(p40) Haplotype 3 (H3); and IL12RB2 locus is IL12RB2 Haplotype 4 (H4).

In various other embodiments, the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP) and denaturing gradient gel electrophoresis (DGGE). In other embodiments, the presence of ten risk haplotypes presents a greater susceptibility than the presence of nine, eight, seven, six, five, four, three, two, one or none of the risk haplotypes, and the presence of nine risk haplotypes presents a greater susceptibility than the presence of eight, seven, six, five, four, three, two, one or none of the risk haplotypes, and the presence of eight risk haplotypes presents a greater susceptibility than the presence of seven, six, five, four, three, two, one or none of the risk haplotypes, and the presence of seven risk haplotypes presents a greater susceptibility than the presence of six, five, four, three, two, one or none of the risk haplotypes, and the presence of six risk haplotypes presents a greater susceptibility than the presence of five, four, three, two, one or none of the risk haplotypes, and the presence of five risk haplotypes presents a greater susceptibility than the presence of four, three, two, one or none of the risk haplotypes, and the presence of four risk haplotypes presents a greater susceptibility than the presence of three, two, one or none of the risk haplotypes, and the presence of three risk haplotypes presents a greater susceptibility than the presence of two, one or none of the risk haplotypes, and the presence of two risk haplotypes presents a greater susceptibility than the presence of one or none of the risk haplotypes, and the presence of one risk haplotype presents a greater susceptibility than the presence of none of the risk haplotypes.

In other embodiments, the detection of risk and/or protective haplotypes is relative to that detected in a healthy subject.

Various embodiments of the present invention provides for a process for stratifying patients comprising: providing a sample from the subject; assaying the sample for risk and/or protective haplotypes from genes selected from the group consisting of: IL23R, IL17A, IL17RA, IL17RD, IL12B(p40), IL and IL12RB2; and a stratifying patients as having a low probability to develop IBD if an increased number of protective haplotypes and a decreased number of risk haplotypes are detected or stratifying patients as having a high probability to develop IBD if a decreased number of protective haplotypes and an increased number of risk haplotypes are detected. In other embodiments, IBD comprises Crohn's Disease (CD) and ulcerative colitis (UC).

In another embodiment, the risk haplotypes located at the: IL23R locus are IL23R Block 2 Haplotype 1 (B2H1) and/or IL23R Block 3 Haplotype 1 (B3H1); IL17A locus are IL17A Haplotype 2 (H2); IL17RA locus is IL17RA Block 2 Haplotype 4 (B2H4); IL17RD locus is IL17RD Block 2 Haplotype 2 (B2H2); IL12RB1 locus is IL12RB1 Haplotype 1 (H1); and IL12RB2 locus are IL12RB2 Haplotype 1 (H1) and/or IL12RB2 Haplotype 3 (H3). In other embodiment, the protective haplotypes located at the: IL23R locus are IL23R Block 2 Haplotype 2 (B2H2), IL23R Block 3 Haplotype 2 (B3H2) and/or IL23R Block 3 Haplotype 6 (B3H6); IL17A locus is IL17A Haplotype 4 (H4); IL17RA locus is IL17RA Block 1 Haplotype 3 (B1H3); IL17RD locus are IL17RD Block 1 Haplotype 2 (B1H2) and/or IL17RD Block 2 Haplotype 3 (B2H3); IL12B(p40) locus are IL12B(p40) Haplotype 1 (H1) and/or IL12B(p40) Haplotype 3 (H3); and IL12RB2 locus is IL12RB2 Haplotype 4 (H4).

In various other embodiments, the detection of IL23R B2H1 and/or IL23R B3H1 in a pediatric non-Jewish subject indicates an increased probability for IBD. In other embodiments, the detection of IL12RB2H4 in a subject of Ashkenazi Jewish descent indicates a decreased susceptibility to IBD and wherein the detection of IL12B2H1 and/or IL12B2H3 in a subject of Ashkenazi Jewish descent indicates an increased susceptibility to IBD. In other embodiments, the detection of IL17A H2 in a non-Jewish subject and the detection of IL17A H4 in a Jewish subject indicates an increased probability for IBD.

In various other embodiments, the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP) and denaturing gradient gel electrophoresis (DGGE). In other embodiments, the presence of ten risk haplotypes presents a greater susceptibility than the presence of nine, eight, seven, six, five, four, three, two, one or none of the risk haplotypes, and the presence of nine risk haplotypes presents a greater susceptibility than the presence of eight, seven, six, five, four, three, two, one or none of the risk haplotypes, and the presence of eight risk haplotypes presents a greater susceptibility than the presence of seven, six, five, four, three, two, one or none of the risk haplotypes, and the presence of seven risk haplotypes presents a greater susceptibility than the presence of six, five, four, three, two, one or none of the risk haplotypes, and the presence of six risk haplotypes presents a greater susceptibility than the presence of five, four, three, two, one or none of the risk haplotypes, and the presence of five risk haplotypes presents a greater susceptibility than the presence of four, three, two, one or none of the risk haplotypes, and the presence of four risk haplotypes presents a greater susceptibility than the presence of three, two, one or none of the risk haplotypes, and the presence of three risk haplotypes presents a greater susceptibility than the presence of two, one or none of the risk haplotypes, and the presence of two risk haplotypes presents a greater susceptibility than the presence of one or none of the risk haplotypes, and the presence of one risk haplotype presents a greater susceptibility than the presence of none of the risk haplotypes.

In other embodiments, the detection of risk and/or protective haplotypes is relative to that detected in a healthy subject.

Various embodiments of the present invention provides for a method of diagnosing susceptibility of IBD in a pediatric non-Jewish subject comprising: providing a sample from the pediatric non-Jewish subject; assaying the sample for the risk haplotypes IL23R B2H1 and/or IL23R B3H1; and determining that the subject has an increased susceptibility to IBD if the risk haplotypes IL23R B2H1 and/or IL23R B3H1 are present or determining that the subject has a decreased susceptibility to IBD if the risk haplotypes IL23R B2H1 and/or IL23R B3H1 are absent. In other embodiments, IBD comprises Crohn's Disease (CD) and ulcerative colitis (UC). In other embodiments, the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP) and denaturing gradient gel electrophoresis (DGGE). In other embodiments, the detection of risk haplotypes is relative to that detected in a healthy subject. In other embodiments, the detection of risk haplotypes is relative to that detected in a healthy subject. In other embodiments, there is a greater susceptibility to IBD when an increased number of risk haplotypes are present and a decreased susceptibility when a decreased number of risk haplotypes are present.

Various embodiments of the present invention provides for a method of diagnosing susceptibility of IBD in subject of Ashkenazi Jewish descent comprising: providing a sample from the subject of Ashkenazi Jewish descent; assaying the sample for the risk haplotypes IL12B2H1 and/or IL12B2H3; and determining that the subject has an increased susceptibility to IBD if the risk haplotypes IL12B2H1 and/or IL12B2H3 are present or determining that the subject has a decreased susceptibility to IBD if the risk haplotypes IL12B2H1 and/or IL12B2H3 are absent. In other embodiments, IBD comprises Crohn's Disease (CD) and ulcerative colitis (UC). In other embodiments, the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP), denaturing gradient gel electrophoresis (DGGE). In other embodiments, the detection of risk haplotypes is relative to that detected in a healthy subject.

In other embodiments, there is a greater susceptibility to IBD when an increased number of risk haplotypes are present and a decreased susceptibility when a decreased number of risk haplotypes are present.

### Variety of Methods and Materials

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a genetic variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well-known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well-known assays described above or another art-recognized genetic assay.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### High Frequency IL23R Haplotypes Explain a High Percentage Risk

The inventors studied the association of IL23R haplotypes with CD and associated serotypes. CD subjects (n=763) and ethnically-matched controls (254) were genotyped for 20 single-nucleotide polymorphisms (SNPs) using Illumina and TaqMan MGB technologies. SNPs were selected to tag Caucasian haplotypes using HapMap data. Serum expression of antibodies was determined by ELISA. Presence of disease, IL23R genotype, and serum antibodies were each determined blinded. Haplotypes were determined with PHASE v2; associations with disease were tested by chi-square and to antibody expression by Wilcoxon.

Three haplotype blocks were observed in the IL23R gene. Block 3 spans the protective SNP R381Q. Associations with both a “risk” haplotype and a “protective” haplotype were observed in Blocks 2 and 3 (Block 2: Risk, 64% in CD, 55% in controls, p=0.015; Protective, 54% in CD, 65% in controls, p=0.005; Block 3: Risk, 64% CD, 56% controls, p=0.015; Protective, 37% CD, 47% controls, p=0.003). Block 2 risk and Block 3 risk are additive for increased risk (ptrend=0.0072) and Block 2 protective and Block 3 protective are additive for decreased risk (ptrend<0.0001). Population attributable risk (PAR) for Block 2 and Block 3 risk is ˜10-20% and is much greater than the PAR for the low frequency R381Q (˜2%). The Block 3 risk haplotype was associated with increased serum expression of anti-I2 antibody (median 12 level for presence of risk haplotype 27.5 compared with 19.6 for absence of risk haplotype, p=0.01).

Thus, IL23R risk haplotypes confer marked, additional CD risks compared with the functional, protective SNP IL21R R381Q. IL23R therefore accounts for a substantial increase in CD risk. Furthermore, IL23R haplotypes are associated with serum expression of antibody to 12, a Pseudomonas related antigen. Subjects with these haplotypes will be important for studying IL23R function. These observations increase the relative importance of this gene in the etiology of CD.

### IL23 Receptor (IL23R) Gene Protects Against Pediatric Crohn's Disease

IL23R has recently been found to be associated with small bowel Crohn's disease (CD) in a large whole genome association study and the rare allele of the R381Q SNP conferred protection against CD. In the IL10-knockout mouse model of colitis, IL23R has been demonstrated to play a role in intestinal inflammation. It is unknown whether IL23R is associated with IBD in children.

The inventors examined the association of IL23R with susceptibility to ulcerative colitis (UC) and CD in pediatric patients. DNA was collected from 610 subjects (152 CD trios, 52 UC trios). Both parents and the affected child were genotyped for the protective R381Q SNP (rs11209026) of the IL23R gene and 4 variants of the CARD15 gene (SNP5, SNP8, SNP12, SNP13) using Taqman technology. The transmission disequilibrium test (TDT) was used to test association to disease using GENEHUNTER 2.0.

The rare allele of R381Q SNP was present in 5.3% of CD and 5.9% UC probands. CARD15 frequency (any variant) was 35% (CD) and 11% (UC). Similar frequencies were observed for parents for both genes. The IL23R allele was negatively associated with IBD: the R381Q SNP was under transmitted in children with IBD (8 transmitted (T) vs. 27 untransmitted (UT); p=0.001). This association was significant for all CD patients (6 T vs. 19 UT; p=0.009), especially for non-Jewish CD patients (2 T vs. 17 UT; p=0.0006). TDT showed a borderline association for UC (T 2 vs. 8 UT; p=0.06). As expected, CARD15 was associated with CD in children by the TDT: (63 T vs. 30 UT p=0.0006), but not with UC (Table 1).

TABLE 1

| | IBD | | | CD | | | UC | | |
|---|---|---|---|---|---|---|---|---|---|
| IL23R rare | | | P | | | P | | | P |
| allele | T | UT | VALUE | T | UT | VALUE | T | UT | VALUE |
| R381Q SNP | 8 | 27 | 0.001 | 6 | 19 | 0.009 | 2 | 8 | P = 0.06 |

T = Transmitted
UT = Undertransmitted

Thus, the CARD15 association acted as a control in this study: the observed association with CARD15 demonstrated that applying the TDT to this pediatric cohort will be useful in further gene finding for IBD. The protective IL23R R381Q variant was particularly associated with CD in non-Jewish children. Thus, the initial whole genome association study based on ileal CD in adults has been extended to the pediatric population and beyond small bowel CD.

Different Haplotypes of the IL12B(p40) Gene are Associated with Clinical Crohn's Disease (CD) and with CD Patients Expressing Cbir1 Antibodies, Respectively

The IL12B gene codes for the p40 subunit shared in common by IL12 and IL23, key cytokines that bridge innate and Th1/Th17 adaptive immune responses. CD has been associated with increased secretion of IL12 and IL23, and treatment with p40 antibody has been effective in certain CD patients. The inventors have previously shown that the antibody response to microbial antigens defines different groups of IBD patients, including those with complicated disease.

The inventors investigated IL12B associations with CD and antibody expression. Four IL12B SNPs: rs3212227 (previously associated with autoimmune disease), F298V, rs2853694 (intron 4), and I33V were genotyped by Illumina GoldenGate Assay in 763 CD patients, and 254 controls. Serum antimicrobial antigens were measured by ELISA. Chi-square was used to test for association of haplotypes with disease and presence of antibody.

One haplotype block was found by Haploview 3.3. Individual haplotypes were obtained by PHASE and ordered by frequency. Among three common haplotypes, haplotype 1 (H1:2212) was negatively associated with CD, i.e. protective (CD vs. control: 68.3% vs. 77.2%, p=0.007), with similar direction in both Jews and non-Jews. The inventors also observed an association between haplotype 3 (H3:1222) and anti-Cbir1 expression in these CD patients, in that H3 frequency was significantly lower in the patients who were anti-Cbir1 positive (31.8% vs. 43.9%, p=0.001). This association was again observed in both Jews and non-Jews.

The inventors have identified one IL12B gene haplotype protective for clinical CD and a different protective haplotype in CD patients who expressed antibody to CBir1. These results support the concept that IL12B variants, and therefore, IL12 and/or IL23 are involved in the overall susceptibility to CD as well as the subtype of CD patients defined by anti-CBir1 expression.

### Association Between IL17A and IL17RA Genes and Inflammatory Bowel Disease

IL17A is produced by TH17 CD4+ T cells, and in some mouse models of colitis, IL17A is responsible for mucosal inflammation. Its role in human IBD is not yet known. IL17RA is a ubiquitously expressed receptor that is essential for IL17A biologic activity. The inventors determined whether IL17A and/or IL17RA genes are associated with IBD. SNPs were selected to tag common Caucasian haplotypes in IL17A (#3605) and IL17RA (#23765) and genotyped in 763 Crohn's disease (CD), 351 ulcerative colitis (UC) and 254 controls using Illumina technology. Analysis was first done in the total sample, and then Haploview 3.3. Individual haplotypes were obtained by PHASE v2 and ordered by frequency (Tables 2 and 3).

TABLE 2
Haplotype of IL17A (1: rare allele)

| SNP | H1 | H2 | H3 | H4 | H5 |
|---|---|---|---|---|---|
| rs2275913 | 2 | 1 | 2 | 1 | 2 |
| rs3819025 | 2 | 2 | 2 | 2 | 1 |
| rs10484879 | 2 | 1 | 2 | 2 | 2 |
| rs7747909 | 2 | 1 | 2 | 2 | 2 |
| rs1974226 | 2 | 2 | 1 | 2 | 2 |

TABLE 3
Haplotype of IL17RA (1: rare allele)

| SNP | H1 | H2 | H3 | H4 | H5 |
|---|---|---|---|---|---|
| Block1: | | | | | |
| rs7288159 | 2 | 1 | 1 | | |
| rs6518660 | 2 | 1 | 2 | | |
| Block2: | | | | | |
| rs2302519 | 1 | 2 | 2 | 2 | 1 |
| rs721930 | 2 | 1 | 2 | 2 | 2 |
| rs2241046 | 2 | 2 | 1 | 2 | 2 |
| rs2241049 | 2 | 1 | 2 | 2 | 1 |
| rs879574 | 2 | 2 | 2 | 1 | 2 |
| rs879577 | 1 | 2 | 2 | 2 | 2 |
| rs882643 | 2 | 2 | 2 | 2 | 1 |

Two major haplotypes (H2 and H4) of IL17A were associated with CD. In non-Jews, CD patients had a higher frequency of H2 (23.7% vs. 18.2%, p=0.03) and a lower frequency of H4 (8.5% vs. 12.3%, P=0.03) when compared with controls; however, an opposite trend was found in the Jewish population for H2 (22.1% vs. 31.4%, P=0.04). Diplotype (i.e. haplogenotype) analysis for IL17A in non-Jews showed a significant trend for odds ratio (OR):H4/no H2 (OR 0.8), other combinations (OR 1), H2/no H4 (OR 1.7, P Mantel-Hanzel=0.004). IL17RA. Two haplotype blocks were identified for IL-17RA. In the total sample, haplotype 3 (H3) in block 1 was negatively associated with both CD and UC when compared with controls (4.0% vs. 8.1%, P<0.0001). In block 2, H4 was positively associated with IBD (14.8% vs. 10.4%, P=0.01). The results were similar in Jews and non-Jews. The combined analysis for the two blocks of IL17RA also displayed a significant trend for increased OR: H3 block 1/no H4 block 2 (OR 0.55), other, (OR 1), H4 no H3 (OR: 1.84, P Mantel-Hanzel <0.0001).

IL17A appears to be an ethnic specific gene for CD, and IL17RA is a gene associated with both CD and UC. As is the case in mouse colitis, this cytokine/receptor pair could be important in the pathogenesis of a subtype of CD.

### An Interaction Between IL-23R and IL-17A and Between IL-23R and IL-17RA Haplotypes is Necessary for Susceptibility to Crohn's Disease

The inventors determined whether an interaction exists between IL-23R and IL-17A/IL-17RA for conferring susceptibility to CD development. SNPs were selected to tag common haplotypes and genotyped in 763 CD and 254 controls using Illumina technology. Haplotype blocks were constructed using Haploview 3.3. Analysis was done in the total sample first, and then in Jewish and non-Jewish subjects separately. Analysis for gene interaction was performed using the Breslow-Day test.

Two IL23R risk haplotypes were identified (IL23R block 3 H1 and block 2 H1) and one each for IL17A (IL17A H2) and IL17RA (IL17RA H4) to confer increased risk for CD. IL23R and IL17A interaction: while the risk haplotype for each gene contributed susceptibility individually, there was no increased risk for disease if either of the two genes' risk haplotypes were absent. IL-23R absent/IL-17A risk (OR 1.04, p=NS); IL-23R risk/IL-17A absent (OR 1.1, p=NS); however, the combination of the risk haplotypes from IL23R with the risk haplotype from IL17A dramatically increased risk for CD (30% in non-Jewish CD vs. 16% of controls, OR 2.4; p for interaction 0.047). IL23R and IL17RA interaction: IL23R absent/IL17RA risk (OR 1.1, p=NS); IL23R risk/IL17RA absent (OR 1.3, p=NS): i.e. no increased risk if a risk haplotype was absent. Yet again the combination dramatically increased risk in the total CD sample (OR 3.0, p for interaction 0.036). IL17A and IL17RA interaction: In contrast, the inventors found no interaction between the IL17A and the IL17RA haplotypes in non-Jewish CD (P=0.4). When all three haplotypes were examined sequentially for interaction, the OR for CD in the non-Jewish population increased from 1 when neither haplotype was present to 3.7 (CI 1.3-10.1, P_{Mantel-Hanzel}=0004) (Table 4).

TABLE 4

| IL23R risk | IL17RA risk | IL17A risk | OR (CI) | P value |
|---|---|---|---|---|
| No | No | No | 1 | 0.004 |
| * | * | * | 1.0(0.7, 1.6) | |
| ** | ** | ** | 1.9(1.1, 3.2) | |
| Yes | Yes | Yes | 3.7(1.3, 10.1) | |

* One risk Haplotype present (of either IL23R, IL17RA or IL-17A),
** Two risk Haplotypes present (of either IL23R, IL17RA or IL-17A)

The inventors' data demonstrate the multiple and likely complex interactions between the individual components of the IL-23/IL-17 axis, which therefore appear to be playing a significant role in CD mucosal inflammation.

As mentioned above, the identities of the IL23R Block 2 markers, their location on the gene and their nucleotide substitutions may be found in FIGS. 4-6; the identities of the IL23R Block 3 markers, their location on the gene and their nucleotide substitutions may be found in FIGS. 4 and 8-9; the identities of the IL17A markers, their location on the gene and their nucleotide substitutions may be found in Table 2, as well as FIG. 14 and the identities of the IL17RA markers, their location on the gene and their nucleotide substitutions may be found in Table 3, as well as FIG. 17.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

### Example 2

### IL23/IL17 Pathway Genes and their Interactions Provide Major Genetic Susceptibility to Crohn's Disease

The inventors tested the hypothesis that haplotypes in genes of the IL23/IL17 pathway contribute to increased susceptibility for CD. 763 CD subjects and 254 controls were genotyped for single nucleotide polymorphisms in the IL23R, IL23R, IL17A, IL17RA, IL12A, IL12B, IL12RB1, and IL12RB2 genes. Genotyping was performed using both Illumina bead array and ABI TaqMan MGB technologies. Common haplotypes, with control frequencies greater than 5%, were assigned using Phase v2 and were tested for association with CD by chi square, with significance assessed using permutation.

The inventors found that haplotypes with increased risk for CD were observed in the IL23R, IL17A, IL17RA genes, and IL12RB1 genes (IL23R, 55% control, 64% CD, p=0.015; IL17A, 32% control, 36% CD, p=0.015; IL17A, 19% control, 27% CD, p=0.003; IL12RB1, 84% control, 90% CD, p=0.004). These haplotypes substantially increase CD risk as seen by a large estimated population attributable risk (PAR, IL23R risk, ˜19%; IL17A risk, ˜16%; IL17RA risk, ˜10%; IL12RB1 risk). The odds ratio for CD increased with the number of risk haplotypes from these 4 genes (OR=1 for 0 or 1 risk haplotype, 1.3 for 2, 2.5 for 3, and 4 for 4 risk haplotypes, p<0.0001). Furthermore, a synergy was observed between IL23R and IL17A, and between IL23R and IL17RA, in that an increased odds ratio (OR) for CD was observed when a risk haplotype from both genes was present (OR ˜1 for the presence of the risk haplotype from IL23R or IL17A and 2.4 for both, p=0.047 for interaction; OR ˜1.1 for IL23R or IL17RA and ˜3 for both, p=0.036 for interaction). Similarly, no interaction between any of the genes tested and NOD2/CARD15 mutations was observed.

The identification of an IL23R risk haplotype with high population frequency and large population attributable risk demonstrates the importance of this gene for CD susceptibility. The observation of associations between CD and IL17A, IL17RA, and IL12RB1 haplotypes suggests that the IL23/IL17 pathway is important for CD pathogenesis and may be a target for therapy. The lack of interaction of IL23/IL17-related risk variants with NOD2/CARD15 mutations suggest that the IL23/IL17 pathway and NOD2/CARD15 act separately to promote CD.

### Subjects

Recruitment of subjects at the Cedars-Sinai Medical Center Inflammatory Bowel Disease center was conducted under the approval of the Cedars-Sinai Medical Center Institutional Review Board. Disease phenotype was assigned using a combination of standard endoscopic, histological, and radiographic features. Ashkenazi Jewish ethnicity was assigned when two or more grandparents were of Ashkenazi Jewish origin.

### Selection of SNPs

SNPs were selected by applying the “Tagger” option in the program Haploview to data from the International HapMap Project. SNPs that “tagged” major Caucasian haplotypes and at the same time were predicted to be compatible with the Illumina genotyping technology using the Illumina Assay Design Tool were genotyped in the initial phases of this study. Since the inventors were interested in major genetic effects for this study rather than rare alleles, the goal of “tagging” was to find a set of tagSNPs in linkage disequilibrium with all SNPs in the HapMap data with a minor allele frequency ≧5%; in some cases this goal was not completely met due to the limitations of the Illumina technology. A few SNPs were also added that were: 1) non-synonymous and had a minor allele frequency greater than 3%, 2) redundant in order to accommodate some assay failure in the initial Illumina run, and 3) markers suggested by information provided by SeattleSNPs. SNPs showing positive associations were selected for further genotyping by ABI technology.

TABLE 5
SNPs Genotyped

| | | | Percent with Minor Allele | | |
|---|---|---|---|---|---|
| | | | Controls | CD | |
| | | TaqMan Assay | N = | N = | |
| dbSNP | Gene | if used | 257 | 753 | p-value |
| rs2853694 | IL12B, | c_2084298_10 | 77.3 | 67.9 | 0.0042 |
| | p40 | | | | |
| rs3212227 | IL12B, | c_2084293_10 | 40.5 | 38.2 | |
| | p40 | | | | |
| rs3213096 | IL12B, | | 0.8 | 1.8 | |
| | p40 | | | | |
| rs3213119 | IL12B, | | 2.8 | 4.6 | |
| | p40 | | | | |
| rs375947 | IL52RB1 | | 55.1 | 56.2 | |
| rs376008 | IL12RB1 | c_795459_10 | 54.7 | 56.4 | |
| rs425548 | IL12RB1 | | 37.6 | 35.8 | |
| rs436657 | IL12RB1 | | 37.6 | 36.7 | |
| rs438421 | IL12RB1 | c_795437_10 | 40.5 | 50.0 | 0.01 |
| rs10484879 | IL17A | custom design | 38.9 | 43.5 | |
| rs1892280 | IL17A | c_12029406_10 | 42.7 | 52.0 | 0.01 |
| rs1974226 | IL17A | custom design | 32.0 | 39.2 | 0.04 |
| rs2275913 | IL17A | c_15979983_10 | 52.8 | 52.0 | |
| rs2894798 | IL17A | | 44.9 | 52.6 | 0.034 |
| rs3819024 | IL17A | | 55.9 | 56.0 | |
| rs3819025 | IL17A | c_292278_10 | 11.2 | 10.3 | |
| rs4711998 | IL17A | | 45.7 | 40.2 | |
| rs7747909 | IL17A | custom design | 39.4 | 44.4 | |
| rs8193036 | IL17A | | 40.9 | 40.9 | |
| rs2041629 | IL17RA | | 30.7 | 34.4 | |
| rs2241042 | IL17RA | | 50.6 | 65.1 | |
| rs2241046 | IL17RA | c_2666438_1_{—} | 36.9 | 36.0 | |
| rs2241048 | IL17RA | | 57.0 | 78.0 | <0.0001 |
| rs2241049 | IL17RA | custom design | 57.1 | 60.1 | |
| rs2302519 | IL17RA | c_15757768_10 | 68.5 | 65.1 | |
| rs6518660 | IL17RA | | 27.6 | 32.2 | |
| rs721930 | IL17RA | c_12689_10 | 33.1 | 38.2 | |
| rs7296159 | IL17RA | | 41.7 | 37.2 | |
| rs879574 | IL17RA | c_11283754_10 | 21.7 | 30.8 | 0.005 |
| rs879575 | IL17RA | c_7620883_10 | 44.4 | 41.2 | |
| rs878577 | IL17RA | c_2666446_20 | 44.9 | 42.4 | |
| rs882643 | IL17RA | c_7620881_10 | 28.4 | 24.8 | |
| rs887786 | IL17RA | | 33.9 | 32.5 | |
| rs9906603 | IL17RA | | 70.9 | 72.4 | |
| rs11171806 | IL23A, | c_25985467_10 | 10.7 | 10.8 | |
| | p19 | | | | |
| rs1004819 | IL23R | c_1272321_10 | 55.4 | 64.0 | 0.015 |
| rs10489628 | IL23R | c_11283754_10 | 63.4 | 56.1 | 0.045 |
| rs11209008 | IL23R | | 7.6 | 4.2 | 0.032 |
| rs11465797 | IL23R | | 11.9 | 8.6 | |
| rs11804284 | IL23R | c_2990003_10 | 19.5 | 20.2 | |
| rs12041056 | IL23R | | 19.6 | 17.7 | |
| rs1343151 | IL23R | c_8367043_10 | 57.7 | 45.2 | |
| rs1569922 | IL23R | | 14.7 | 7.0 | 0.0000 |
| rs1684444 | IL23R | | 89.7 | 68.0 | |
| rs2201841 | IL23R | | 55.8 | 64.4 | 0.014 |
| rs2863212 | IL23R | | 16.7 | 16.7 | |
| rs6671221 | IL23R | | 69.7 | 68.0 | |
| rs752892 | IL23R | c_2990015_10 | 37.5 | 39.5 | |
| rs7530511 | IL23R | c_2990018_10 | 19.9 | 20.8 | |
| rs790631 | IL23R | c_1272311_10 | 46.4 | 49.6 | |

### Genotyping

DNA was isolated from Epstein Barr virus transformed lymphoblastoid cell lines using proteinase K digestion, organic extraction, and ethanol precipitation. Single nucleotide markers (SNPs) were genotyped using one of two methods: (1) the oligonucleotide ligation assay, Illumina Golden Gate technology, following the manufacturer's protocol (Illumina, San Diego, Calif.), and (2) the 5′-extension reaction, TaqMan MGB technology, following the manufacturer's protocol (Applied Biosystems, Bulletin #4322856). Consistency of SNP genotyping between the two methods was checked for each SNP by genotyping 100 samples with both methods.

### Statistical Analyses

Haplotype blocks were determined using the “Tagger” routine of the program Haploview. Haplotypes of subjects were inferred from the genotyping data using the program PHASE v2. The association of the presence of a haplotype was tested using the chi-square test and the significance of results was assessed by applying a permutation test to the data in order to correct for multiple testing due to the number of haplotypes. Results with significance were defined by p<0.05 by permutation test. Due to sample size considerations, the results reported are for all CD and control subjects with Jewish and non-Jewish subjects combined. The notable exception to this is that an IL17A “risk” haplotype specific to the non-Jewish population was identified in the hypothesis-generating phase of this study and used for subsequent gene-gene interaction studies. Population attributable risk was estimated by assuming that 1) the frequency of a particular haplotype in the controls reflected the population frequency of that haplotype, and 2) the odds ratio for the association of a given haplotype reflected the relative risk of that haplotype for Crohn's disease. For this report, haplotypes are numbered in order of frequency in controls (H1, H2, and so forth) and the nucleotides for each tagSNP are listed in Table 5 according to the forward strand of the NCBI human genome build 36 and dbSNP. A “major” haplotype in this report is a haplotype with a population frequency greater than 5% in the controls.

### TagSNPs Selected in Genes Related to the IL12/IL23 Pathway

TagSNPs were first selected for the major Caucasian haplotypes in eight genes related to the IL12/IL23 pathway (Table 6), genotyped in a CD case-control cohort, used to infer haplotypes, and then tested for association with Crohn's disease.

TABLE 6
Genes related to the IL12/IL23 pathway

| GENE | GENE | |
|---|---|---|
| ID* | ABBREVIATION | GENE DESCRIPTION |
| 3592 | IL12A | Interleukin 12A (natural killer cell |
| | | stimulatory factor 1, cytotoxic |
| | | lymphocyte maturation factor 1, p35) |
| 3593 | IL12B | Interleukin 12B (natural killer cell |
| | | stimulatory factor 2, cytotoxic |
| | | lymphocyte maturation factor 2, p40) |
| 3594 | IL12RB1 | Interleukin 12 receptor beta 1 |
| 3595 | IL12RB2 | Interleukin 12 receptor beta 2 |
| 3605 | IL17A | Interleukin 17A |
| 23765 | IL17RA | Interleukin 17 receptor A |
| 51561 | IL23A | Interleukin 23 alpha subunit p19 |
| 148233 | IL23R | Interleukin 23 receptor |

*Gene ID from dbGene of the National Center for Biotechnology Information, National Library of Medicine, National Institutes of Health.

### IL23R

IL23R haplotypes with high population frequency were observed to be associated with CD. Three IL23R haplotype blocks were inferred from tagSNP data. No associations between CD and IL23R Block 1 haplotypes were observed. However, CD was associated with the individual SNP rs1569922, located between Block 1 and Block 2 (85% in controls compared with 93% in CD subjects, p<0.0001) as well as haplotypes in blocks 2 and 3. Haplotypes that both increased CD risk (“risk,” IL23R Block 2 H1 and IL23R Block 3 H1) and decreased CD risk (“protective,” IL23R Block 2 H2 and IL23R Block 3 H2) were observed. Furthermore, within each block, the odds ratio for CD was increased with the number of copies of the “risk” haplotype and was decreased with the number of copies of the “protective” haplotype from 0 through 1 to 2 copies (“risk:” IL23R Block 2 H1, p(trend)=0.0091, IL23R Block 3 H1, p(trend)=0.0097; “protective:” IL23R Block 2 H2, p(trend)=0.0002, Block 2 H2, p(trend)=0.0011). The odds ratio for CD risk was increased with the number of “risk” haplotypes from both haplotype blocks (“risk,” p(trend)=0.0072; “protective,” p(trend)<0.0001). In this study, the IL23R functional and “protective” allele (R381Q, rs11209026) was located on IL23R Block 3 H6. The magnitude of the population attributable risk, or the amount of the disease that would not exist if a risk factor is removed from a population, was much greater for the presence of the “risk” or the “protective” haplotypes reported here than for the previously reported IL23R Block 3 H6 containing IL23R R381Q (˜20% for the presence of either the “risk” haplotypes together or the “protective” haplotypes together and ˜4% for the presence of Block 3 H6).

### IL17A

The tagSNPs formed one haplotype block spanning most of this gene. When all subjects were considered, IL H4 was “protective,” conferring a decreased risk for CD (frequency in controls 20.5%, in CD 13.5%, p=0.007). When only non-Jewish subjects were considered, IL17A H4 remained “protective” (Controls, 24.1%, CD 16.0%, p=0.014) and IL17A H2 was a “risk” haplotype, conferring increased risk for CD (Controls, 32.0%, CD, 42.1%, p=0.015). These IL17A haplotypes were associated with a substantial risk for CD in non-Jewish subjects; the magnitude of the population attributable risk was ˜16% for IL17A H2 (“risk”) in non-Jewish subjects and minus ˜10% for IL17A H4 (“protective”).

### IL17RA

The tagSNPs formed two haplotype blocks. IL17RA Block 1 H3 was associated with a decreased susceptibility for CD (“protective,” Controls 15.8%, CD 7.5%, p<0.0000) and IL17RA Block 2 H4 was associated with an increased susceptibility for CD (Controls, 18.9%, CD, 27.0%, p=0.01). The magnitude of the population attributable risk for IL17RA Block 2 H4 was ˜10% and for Block 1 H3 was minus ˜3%.

### IL12B, IL12RB1, IL12RB2

For IL12B, the tagSNPs formed one haplotype block and H1 was associated with a modestly decreased susceptibility for CD (“protective,” Controls, 77.2%, CD 68.3%, p=0.004) and a population attributable risk of minus ˜28%. For IL12RB1, the tagSNPs formed one haplotype block and H1 was associated with a greater susceptibility for CD (“risk,” Control, 83.5%, CD, 90.2%, p=0.004). For IL12RB2, the tagSNPs formed one haplotype block and H4 was associated with a modest decrease in susceptibility for CD (“protective,” Control, 24.3%, CD, 18.5%, p=0.036). In contrast to the other observed associations, this association of CD and IL12RB2 haplotypes was particular to Ashkenazi Jewish subjects because when Ashkenazi Jewish and non-Jewish CD subjects were analyzed separately, the association of CD and the IL12RB2 H4 protective haplotype was observed in the Jewish subjects only (Jewish: Control. 43.4%, CD 21.9%, p=0.001; non-Jewish: Control, 19.4%, CD, 16.1%, p is not significant). Furthermore, a significant risk haplotype for this population was also observed, the presence of IL12RB2 H1 (Jewish: Control, 62.3%, CD, 78.6%, p=0.009; non-Jewish: Control, 82.5%, CD, 79.4%, p not significant).

IL12A (p35) and IL23 (p19)

No association was observed between CD and haplotypes formed by 4 IL12A tagSNP's nor between IL23 tagSNP rs11171806.

### Interactions Between IL23R, IL17A, IL17RA, and IL12RB1

One hypothesis was that the combination of variations in genes related to the IL23/IL17 pathway contributes to increased risk of CD. Therefore the inventors analyzed combinations of the risk and protective haplotypes observed to increase CD susceptibility individually (Table 7).

TABLE 7
(a-b): Gene-Gene Interactions Between IL23R, IL17A, and IL17RA
(a)

| Presence | | | | | | | |
|---|---|---|---|---|---|---|---|
| of | | | | | | | |
| IL23R | | | | | | | |
| Block 2 | | | | | | | |
| H1 or | Presence | | | | | | Inter- |
| IL23R | of | | | | | Mantel- | action |
| Block 3 | IL17A | | | Odds | 95% | Haenszel | P |
| H1 | H2 | CD | Control | Ratio | CI | P value | value |
| No | No | 90 | 52 | 1 | | 0.0017 | 0.047 |
| No | Yes | 52 | 30 | 1.0 | 0.6-1.8 | | |
| Yes | No | 166 | 84 | 1.1 | 0.7-1.8 | | |
| Yes | Yes | 133 | 32 | 2.4 | 1.4-4.0 | | |

(b)

| Presence | | | | | | | |
|---|---|---|---|---|---|---|---|
| of IL23R | | | | | | | |
| Block 2 | | | | | | | |
| H1 or | Presence | | | | | | Inter- |
| IL23R | of | | | | | Mantel- | action |
| Block 3 | IL17RA | | | Odds | 95% | Haenszel | P |
| H1 | H4 | CD | Control | Ratio | CI | P value | value |
| No | No | 175 | 78 | 1 | | 0.0003 | 0.036 |
| No | Yes | 65 | 27 | 1.1 | 0.6-1.8 | | |
| Yes | No | 370 | 126 | 1.3 | 0.9-1.8 | | |
| Yes | Yes | 138 | 20 | 3.0 | 1.8-5.2 | | |

First, IL23R and IL17A variation interacted to increase CD susceptibility. When the IL23R and IL17A “risk” haplotypes were present together, the odds ratio for CD increased substantially over the odds ratio for CD when either “risk” haplotype was present alone (IL23R H1 from either Block 2 or 3 present and IL17A H4 present, OR=2.4, compared with 1.0-1.1 when either one “risk” haplotype was present or no “risk” haplotype was present, p(Mantel-Haenszel)=0.0017, p(logistic regression test for interaction)=0.047). The Mantel-Haenszel analysis suggested that the trend from no “risk” haplotypes through one to two is significant while the logistic regression analysis for interaction suggested that the two risk haplotypes synergistically interacted to increase CD susceptibility.

Second, IL23R and IL17RA variation also interacted to increase CD susceptibility. When the IL23R and IL17RA risk haplotypes were present together, the odds ratio for CD increased over the odds ratio when either “risk” haplotype was present alone (IL23R H1 from either Block 2 or 3 present and IL17RA H4 present, OR=3.0, compared with 1.0-1.3 when either one “risk” haplotype or no “risk” haplotype was present, p(Mantel-Haenszel)=0.0003, p(logistic regression for interaction)=0.036). Again, the Mantel-Haenszel analysis suggested that the trend from no “risk” haplotype through one to two is significant while the logistic regression analysis suggested that the two risk haplotypes synergistically interacted to increase CD susceptibility.

Third, but in contrast, IL17A and IL17RA variation was additive for each but with no interaction. The odds ratio for CD when both IL17A and IL17RA “risk” haplotypes were present was not greater than the odds ratio for CD when only one of the IL17A or IL17RA “risk” haplotypes was present (IL17A H2 and IL17RA H4 present, OR=1.7, either IL17A H2 or IL17RA H4 present, OR=1.5-1.7, no IL17A or IL17RA “risk” haplotype present, OR=1.0, p(Mantel-Haenszel)=0.005, p(logistic regression for interaction) was not significant). The Mantel-Haenszel analysis suggested that the presence of either IL17A or IL17RA “risk” haplotype significantly increased CD susceptibility, but the non-significant logistic regression analysis suggested that variants in these two genes were not interacting to increase CD susceptibility.

Combining the risk haplotypes from IL23R, IL17A, IL17RA, and IL12RB1 in a single analysis showed a significant increase in the odds ratio for CD from no “risk” haplotype to 3 “risk” haplotypes (OR for CD is 1, 1.1, 1.3, 2.5, and 3.7 for 0, 1, 2, 3, 4 “risk” haplotypes, respectively, p(Mantel-Haenszel)<0.0001). This analysis demonstrated that IL23R, IL17A, IL17RA, and IL12RB1 genetic variation contributes substantially to CD susceptibility.

Interaction with CARD15 Mutations

Since a recent genome-wide association study observed that CARD15 and IL23R were the two greatest contributors to CD risk, the interaction between three common CARD15 mutations and IL23/IL17 haplotypes was examined (Table 8). CD susceptibility was significantly increased when one CARD15 “risk” mutation was present with one of the IL23R, IL17A, and IL17RA “risk” haplotypes (p-values for Mantel-Haenszel tests were significant). However, when tested for interaction, the presence of a CARD15 mutation did not interact with the presence of one of the IL23R, IL17A or IL17RA “risk” haplotypes (p-values for the interaction test were not significant).

TABLE 8
Interactions between CARD 15 mutations and IL23R, IL17A, and IL17RA “risk” haplotypes
a) CARD15 and IL23R in all subjects

| | Presence of | | | | | | |
|---|---|---|---|---|---|---|---|
| Presence of | IL23R Block | | | | | | Inter- |
| at least 1 | 2 H1 or | | | | 95% | Mantel- | action |
| CARD15 | IL23R Block 3 | | | Odds | Confidence | Haenszel | P |
| mutation* | H1 | CD | Control | Ratio | Interval | P value | value |
| No | No | 150 | 97 | 1 | | <0.0001 | 0.07 |
| Yes | No | 90 | 8 | 7.3 | 3.4-15.7 | | |
| No | Yes | 339 | 126 | 1.7 | 1.3-2.4 | | |
| Yes | Yes | 167 | 20 | 5.4 | 3.2-9.2 | | |

b) CARD15 and IL17A in non-Jewish subjects

| Presence of | | | | | | | Inter- |
|---|---|---|---|---|---|---|---|
| at least 1 | | | | | 95% | Mantel- | action |
| CARD15 | Presence of | | | Odds | Confidence | Haenszel | P |
| mutation | IL17A H2 | CD | Control | Ratio | Interval | P value | value |
| No | No | 159 | 120 | 1 | | <0.0001 | 0.5 |
| Yes | No | 101 | 18 | 4.2 | 2.4-7.4 | | |
| No | Yes | 131 | 59 | 1.7 | 1.1-2.6 | | |
| Yes | Yes | 58 | 4 | 10.9 | 3.9-31 | | |

c) CARD15 and IL17R in all subjects

| Presence of at | | | | | | | Inter- |
|---|---|---|---|---|---|---|---|
| least 1 | | | | | 95% | Mantel- | action |
| CARD15 | Presence of | | | Odds | Confidence | Haenszel | P |
| mutation | IL17A H2 | CD | Control | Ratio | Interval | P value | value |
| No | No | 361 | 181 | 1 | | <0.0001 | 0.6 |
| Yes | No | 196 | 25 | 3.9 | 2.5-6.2 | | |
| No | Yes | 140 | 44 | 1.6 | 1.1-3.2 | | |
| Yes | Yes | 66 | 4 | 8.3 | 3.0-23.0 | | |

*CARD 15 mutations are commonly known as SNP8 (CARD15 R702W; rs2066844), SNP12 (G908R; rs2066845); and SNP13 (L1007fsinsC; rs2066647).

### Role of Th17 Cell in Crohn's Disease Pathogenesis

The significant genetic associations and high population attributable risks reported here support the hypothesis that genes in the IL23R/IL17 pathway, individually and in interaction, are major contributors to the genetic susceptibility of Crohn's disease (CD). Since increasing evidence implicates this pathway in the proliferation and subsequent action of the Th17 cell, these results suggest a role for this cell type in CD pathogenesis.

The association of CD with ten IL23R single nucleotide polymorphisms (SNPs), in particular rs11209026 (Arg381Gln), was observed in a whole genome association study of ileal CD; the inventors have confirmed this finding in a pediatric cohort. These observations support the concept that the IL23R gene is a genetic determinant of CD. However, based on the low frequency of the minor allele of IL23R Arg381Gln in the general population, the population attributable risk (PAR) for this allele would be on the order of ˜4% (Control, 7%, CD 1.9%). The “risk” and “protective” IL23R haplotypes reported here are at a much higher frequency in the general population, substantially raising the estimate of the PAR for the IL23R gene to the order of ˜20%. These considerations support the concept that the IL23R gene is a major genetic determinant of CD, on the order of the presence of a CARD15/NOD2 mutation (Table 9).

TABLE 9
Cumulative Population Attributable Risk (PAR) for CD
a) Odds Ratio and Population Attributable
Risk for Carriers of Risk Haplotype

| Gene | OR | PAR |
|---|---|---|
| IL23R | 1.5 | 23 |
| IL17A | 1.6 | 16 |
| IL17RA | 1.6 | 16 |
| NOD2 | 4.2 | 26 |

b) Population Attributable Risk for Carriers
of Two Risk Haplotypes, Pairwise

PAR

| | | Risk Haplotype | |
|---|---|---|---|
| | | from Either | Risk Haplotype |
| | | or Both | from Both |
| Gene 1 | Gene 2 | Genes Present | Genes Present |
| IL23R | IL17A | 22 | 15 |
| IL23R | IL17RA | 25 | 8 |
| IL23R | NOD2 | 48 | 1 |
| IL17A | IL17RA | 21 | 0.1 |
| IL17A | NOD2 | 41 | 2 |
| IL17RA | NOD2 | 34 | 2 |

In addition to IL23R, associations were also observed between CD and common haplotypes in other genes in the IL23/IL17 pathway: IL17A, IL17RA, IL12B, and IL12RB1. A “risk” haplotype, conferring a greater susceptibility to CD, or a “protective” haplotype, conferring a reduced susceptibility to CD, or both, was observed in each of these genes. Both were observed with IL17A and with IL17RA in non-Jewish CD subjects, with PAR on the order of ˜10%; an IL12B protective haplotype was also observed with PAR on the order of ˜28%. Furthermore, risk haplotypes of IL23R and IL17A and of IL23R and IL17RA interacted to increase CD risk only when both were present, supporting the concept that CD pathophysiology involves the products of these genes together. Further support for this concept was the observation of increasing odds ratio for CD as the risk haplotypes for these genes were combined.

While additive to increase CD risk, no interactions between mutations in CARD15/NOD2 and risk haplotypes in the IL23/IL17 pathway were observed. This observation suggests that CARD15/NOD2 and IL23/IL17 variants define two separate pathways to intestinal inflammation. Extensive work with mouse models of intestinal inflammation, developed by “knocking out” many different immune-related genes, has demonstrated that there are multiple genetic pathways to intestinal inflammation. If so, then variation in IL23/IL17 related genes may be useful to distinguish CD subtypes with different underlying pathophysiological mechanisms, and suggests that therapies targeted at IL23/IL17 successfully treat IL23/IL17 pathway-related CD subtypes.

### Example 3

Previous evidence has shown that the IL23-IL17 pathway is important in pathogenesis of Crohn's disease (CD) and that IL23-IL17 pathway genes including IL12b, IL12RB1, IL12RB2, IL17A, IL17RA are associated with CD. IL17RD, another member of the IL17 receptor family, has been detected in various cells, but its role in human CD has been previously unclear. The inventors determined whether IL17RD is associated with CD and whether there is a gene-gene interaction within IL23-IL17 pathway genes. 763 CD subjects and 254 controls were genotyped for single nucleotide polymorphisms in the IL23A, IL23R, IL17A, IL17RA, IL12B, IL12RB1, IL12RB2 and IL17RD genes using Illumina and ABI platforms. Haplotypes were assigned using Phase v2 and were tested for association with CD by chi square test. The inventors utilized multidimensionality reduction (MDR) to explore gene-gene interactions. Two Blocks (B) of IL17RD were found to be associated with CD. CD patients had a higher frequency of haplotype2 in block2 (B2H2, 55.0% vs. 45.4%, OR=1.5, p=0.01) and a lower frequency of B1H2 (39.1% vs. 50.2%, OR=0.64, p=0.002) and B2H3 (37.8% vs. 47.4%, OR=0.68, p=0.01) when compared with controls. Haplotypes with increased risk for CD were observed in the IL23R_B2H1 and B3H1, IL17A_H2, IL17RA_B2H4, IL12RB1_H1 and IL12RB2_H3; haplotypes with decreased risk were observed in the IL23R_B2H2 and B3H2, IL17A_H4, IL17RA_B1H3, IL12B_H1 and IL12RB2_H4. MDR analysis suggested interaction between IL23R_B2H2, IL12RB2_H4 and IL17RD_B2H2 (CV consistency 10/10, tested accuracy 59.7%, p=0.002). The following logistic regression analysis confirmed the interaction (IL23R_B2H2*IL12RB2_H4, p<0.0001; IL23R_B2H2*IL17RD_B2H2, p=0.02). Thus, the inventors have shown that IL17RD is significantly associated with CD and is likely to interact with IL23R in the risk of developing CD.

TABLE 10
Negative Association of IL17-IL23 pathway-
related SNPs with Crohn's Disease

| | | | | | Percent | |
|---|---|---|---|---|---|---|
| | | | | | with | |
| | | | | | Minor | |
| | | | | | Allele | |
| | | | | TaqMan | Controls | CD |
| | | | | Assay | N = | N = |
| db SNP | Gene(s) | Chr | Position | (if used) | 257 | 753 |
| rs475825 | IL12A, | 3 | 161, 193, | C__2936113_10 | 29.3 | 32.9 |
| | p35 | | 022 | | | |
| rs583911 | IL12A, | 3 | 161, 193, | C__2936112_10 | 71.0 | 67.2 |
| | p35 | | 084 | | | |
| rs2243130 | IL12A, | 3 | 161, 193, | C__2936111_10 | 7.0 | 11.0 |
| | p35 | | 686 | | | |
| rs2243149 | IL12A, | 3 | 161, 198, | C__2936107_10 | 64.7 | 63.7 |
| | p35 | | 406 | | | |
| rs2254073 | IL17C | 16 | 87, 233, | | 27.6 | 25.3 |
| | | | 305 | | | |
| rs7985552 | IL17D | 13 | 20, 178, | | 20.6 | 19.1 |
| | | | 738 | | | |
| rs6490604 | IL17D | 13 | 20, 182, | | 26.4 | 24.6 |
| | | | 828 | | | |
| rs9579932 | IL17D | 13 | 20, 188, | | 11.0 | 10.7 |
| | | | 644 | | | |
| rs7787 | IL17D | 13 | 20, 195, | | 50.4 | 48.9 |
| | | | 198 | | | |
| rs721430 | IL17F | 6 | 52, 210, | | 35.1 | 37.1 |
| | | | 599 | | | |
| rs11465551 | IL17F | 6 | 52, 211, | | 7.7 | 8.6 |
| | | | 823 | | | |
| rs7771511 | IL17F | 6 | 52, 212, | | 3.5 | 4.9 |
| | | | 141 | | | |
| rs12201582 | IL17F | 6 | 52, 212, | | 18.2 | 17.4 |
| | | | 648 | | | |
| rs12203582 | IL17F | 6 | 52, 213, | | 42.4 | 40.3 |
| | | | 516 | | | |
| rs1266828 | IL17F | 6 | 52, 216, | | 25.5 | 24.9 |
| | | | 021 | | | |
| rs455863 | IL17RE/ | 3 | 9, 931, | | 48.1 | 47.5 |
| | IL17RC | | 279 | | | |
| rs8883 | IL17RE/ | 3 | 9, 932, | | 48.1 | 46.8 |
| | IL17RC | | 898 | | | |
| rs4686383 | IL17RE/ | 3 | 9, 934, | | 17.7 | 18.5 |
| | IL17RC | | 713 | | | |
| rs708567 | IL17RE/ | 3 | 9, 935, | | 48.1 | 47.4 |
| | IL17RC | | 070 | | | |
| rs7627880 | IL17RE/ | 3 | 9, 944, | | 45.7 | 45.9 |
| | IL17RC | | 328 | | | |
| rs279545 | IL17RE/ | 3 | 9, 947, | | 19.0 | 20.0 |
| | IL17RC | | 494 | | | |
| rs11171806 | IL23A, | 5 | 55, 019, | C__25985467_10 | 10.7 | 10.8 |
| | p19 | | 798 | | | |

TABLE 11
Synergistic interaction between IL23R and IL17RA
Interaction between IL23R risk haplotypes and
IL17RA risk haplotype in all subjects.

| Presence of | Presence | | | | 95% | | |
|---|---|---|---|---|---|---|---|
| IL23R Block 2 | of | | | | Con- | Mantel- | Inter- |
| H1 or IL23R | IL17RA | | Con- | Odds | fidence | Haenszel | action |
| Block 3 H1 | H4 | CD | trol | Ratio | Interval | P value | P value |
| No | No | 175 | 78 | 1 | | 0.0003 | 0.036 |
| No | Yes | 65 | 27 | 1.1 | 0.6-1.8 | | |
| Yes | No | 370 | 126 | 1.3 | 0.9-1.8 | | |
| Yes | Yes | 138 | 20 | 3.0 | 1.8-5.2 | | |

TABLE 12
Interaction between IL23R, IL12RB2, and IL17RD -
Multifactor dimensionality analysis (MDR)

| | balanced | CV | |
|---|---|---|---|
| Model | accuracy | consistency | P |
| IL23R Block 2 Haplotype 2 | 0.4902 | 4/10 | ns |
| IL23R B2H2, IL12RB2 Haplotype 4 | 0.5667 | 9/10 | 0.06 |
| IL23R B2H2, IL12RB2 H4, | 0.5967 | 10/10 | 0.002 |
| IL17RD Block 2 H2 | | | |

TABLE 13
Interaction between IL23R, IL12RB2, and IL17RD -
Further test of MDR model by logistic regression

| parameter | estimate | P |
|---|---|---|
| IL23R Block 2 Haplotype 2 | −1.17 | <0.0001 |
| IL12RB2 Haplotype 4 | −1.35 | <0.0001 |
| IL17RD Block 2 Haplotype 2 | −1 | NS |
| IL23R B2H2 * IL12RB2 H4 | 1.49 | <0.0001 |
| IL23R B2H2 * IL17RD B2H2 | 0.75 | 0.02 |

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention. Furthermore, one of skill in the art would recognize that the invention can be applied to various inflammatory conditions and disorders and autoimmune diseases besides that of inflammatory bowel disease. It will also be readily apparent to one of skill in the art that the invention can be used in conjunction with a variety of phenotypes, such as serological markers, additional genetic variants, biochemical markers, abnormally expressed biological pathways, and variable clinical manifestations.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Accordingly, the invention is not limited except as by the appended claims.

What is claimed is:

1. A method of diagnosing susceptibility to Inflammatory Bowel Disease (IBD) in a subject in need thereof, comprising:
Providing a sample from the subject;
Assaying the sample to detect risk and/or protective haplotypes in one or more genes selected from the group consisting of: IL23R, IL17A, IL17RA, IL17RD, IL12B(p40), IL12RB1 and IL12RB2; and
Determining that the subject has an increased susceptibility to IBD if one or more risk haplotypes are present and the protective haplotypes are absent or determining that the subject has a decreased susceptibility to IBD if one or more protective haplotypes are present and the risk haplotypes are absent.

2. The method of claim 1, wherein IBD comprises Crohn's Disease (CD) and ulcerative colitis (UC).

3. The method of claim 1, wherein the risk haplotypes located at the:
IL23R locus are IL23R Block 2 Haplotype 1 (B2H1) and/or IL23R Block 3 Haplotype 1 (B3H1);
IL17A locus are IL17A Haplotype 2 (H2) and/or IL17A Haplotype 4 (H4);
IL17RA locus is IL17RA Block 2 Haplotype 4 (B2H4);
IL17RD locus is IL17RD Block 2 Haplotype 2 (B2H2);
IL12RB1 locus is IL12RB1 Haplotype 1 (H1); and
IL12RB2 locus are IL12RB2 Haplotype 1 (H1), IL12RB2 Haplotype 3 (H3) and/or IL12RB2 Haplotype 4 (H4).

4. The method of claim 1, wherein the protective haplotypes located at the:
IL23R locus are IL23R Block 2 Haplotype 2 (B2H2), IL23R Block 3 Haplotype 2 (B3H2) and/or IL23R Block 3 Haplotype 6 (B3H6);
IL17RA locus is IL17RA Block 1 Haplotype 3 (B1H3);
IL17RD locus are IL17RD Block 1 Haplotype 2 (B1H2) and/or IL17RD Block 2 Haplotype 3 (B2H3);
IL12B(p40) locus are IL12B(p40) Haplotype (H1) and/or IL12B(p40) Haplotype (H3); and
IL12RB2 locus is IL12RB2 Haplotype 4 (H4).

5. The method of claim 1, wherein the detection of IL23R B2H1 and/or IL23R B3H1 haplotype in a pediatric non-Jewish subject indicates an increased susceptibility to IBD.

6. The method of claim 1, wherein the detection of IL12RB2 H4 haplotype in a subject of Ashkenazi Jewish descent indicates a decreased susceptibility to IBD and wherein the detection of IL12B2H1 haplotype and/or IL12B2H3 haplotype in a subject of Ashkenazi Jewish descent indicates an increased susceptibility to IBD.

7. The method of claim 1, wherein the detection of IL17A H2 haplotype in a non-Jewish subject or the detection of IL17A H4 haplotype in a Jewish subject indicates an increased susceptibility to IBD.

8. The method of claim 1, wherein the detection of IL12B(p40) H3 haplotype and Cbir1 antibody expression indicates a decreased susceptibility to UC in a subject.

9. The method of claim 1, wherein the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP) and denaturing gradient gel electrophoresis (DGGE).

10. The method of claim 1, wherein the detection of risk and/or protective haplotypes is relative to that detected in a healthy subject.

11. The method of claim 1, wherein the presence of ten risk haplotypes presents a greater susceptibility than the presence of nine, eight, seven, six, five, four, three, two, one or none of the risk haplotypes, and the presence of nine risk haplotypes presents a greater susceptibility than the presence of eight, seven, six, five, four, three, two, one or none of the risk haplotypes, and the presence of eight risk haplotypes presents a greater susceptibility than the presence of seven, six, five, four, three, two, one or none of the risk haplotypes, and the presence of seven risk haplotypes presents a greater susceptibility than the presence of six, five, four, three, two, one or none of the risk haplotypes, and the presence of six risk haplotypes presents a greater susceptibility than the presence of five, four, three, two, one or none of the risk haplotypes, and the presence of five risk haplotypes presents a greater susceptibility than the presence of four, three, two, one or none of the risk haplotypes, and the presence of four risk haplotypes presents a greater susceptibility than the presence of three, two, one or none of the risk haplotypes, and the presence of three risk haplotypes presents a greater susceptibility than the presence of two, one or none of the risk haplotypes, and the presence of two risk haplotypes presents a greater susceptibility than the presence of one or none of the risk haplotypes, and the presence of one risk haplotype presents a greater susceptibility than the presence of none of the risk haplotypes.

12. A process for selecting a therapy for a subject susceptible to IBD comprising:
Providing a sample from the subject;
Assaying the sample to detect risk and/or protective haplotypes from genes selected from the group consisting of: IL23R, IL17A, IL17RA, IL17RD, IL12B(p40), IL12RB1 and IL12RB2;
Determining that the subject has an increased susceptibility to IBD if one or more risk haplotypes are present and the protective haplotypes are absent or determining that the subject has a decreased susceptibility to IBD if one or more protective haplotypes are present and the risk haplotypes are absent; and
Selecting a preventative therapy for a subject with an increased susceptibility to IBD.

13. The process of claim 12, wherein IBD comprises Crohn's Disease (CD) and ulcerative colitis (UC).

14. The process of claim 12, wherein the risk haplotypes located at the:
IL23R locus are IL23R Block 2 Haplotype 1 (B2H1) and/or IL23R Block 3 Haplotype 1 (B3H1);
IL17A locus are IL17A Haplotype 2 (H2) and/or IL17A Haplotype 4 (H4);
IL17RA locus is IL17RA Block 2 Haplotype 4 (B2H4);
IL17RD locus is IL17RD Block 2 Haplotype 2 (B2H2);
IL12RB1 locus is IL12RB1 Haplotype 1 (H1); and
IL12RB2 locus are IL12RB2 Haplotype 1 (H1), IL12RB2 Haplotype 3 (H3) and/or IL12RB2 Haplotype 4 (H4).

15. The process of claim 12, wherein the protective haplotypes located at the:
IL23R locus are IL23R Block 2 Haplotype 2 (B2H2), IL23R Block 3 Haplotype 2 (B3H2) and/or IL23R Block 3 Haplotype 6 (B3H6);
IL17RA locus is IL17RA Block 1 Haplotype 3 (B1H3);
IL17RD locus are IL17RD Block 1 Haplotype 2 (B1H2) and/or IL17RD Block 2 Haplotype 3 (B2H3);
IL12B(p40) locus are IL12B(p40) Haplotype (H1) and/or IL12B(p40) Haplotype (H3); and
IL12RB2 locus is IL12RB2 Haplotype 4 (H4).

16. The process of claim 12, wherein the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP) and denaturing gradient gel electrophoresis (DGGE).

17. The process of claim 12, wherein the presence of ten risk haplotypes presents a greater susceptibility than the presence of nine, eight, seven, six, five, four, three, two, one or none of the risk haplotypes, and the presence of nine risk haplotypes presents a greater susceptibility than the presence of eight, seven, six, five, four, three, two, one or none of the risk haplotypes, and the presence of eight risk haplotypes presents a greater susceptibility than the presence of seven, six, five, four, three, two, one or none of the risk haplotypes, and the presence of seven risk haplotypes presents a greater susceptibility than the presence of six, five, four, three, two, one or none of the risk haplotypes, and the presence of six risk haplotypes presents a greater susceptibility than the presence of five, four, three, two, one or none of the risk haplotypes, and the presence of five risk haplotypes presents a greater susceptibility than the presence of four, three, two, one or none of the risk haplotypes, and the presence of four risk haplotypes presents a greater susceptibility than the presence of three, two, one or none of the risk haplotypes, and the presence of three risk haplotypes presents a greater susceptibility than the presence of two, one or none of the risk haplotypes, and the presence of two risk haplotypes presents a greater susceptibility than the presence of one or none of the risk haplotypes, and the presence of one risk haplotype presents a greater susceptibility than the presence of none of the risk haplotypes.

18. The process of claim 12, wherein the detection of risk and/or protective haplotypes is relative to that detected in a healthy subject.

19. A process for stratifying patients comprising:
Providing a sample from the subject;
Assaying the sample for risk and/or protective haplotypes from genes selected from the group consisting of: IL23R, IL17A, IL17RA, IL17RD, IL12B(p40), IL12RB1 and IL12RB2; and
Stratifying patients as having a low probability to develop IBD if an increased number of protective haplotypes and a decreased number of risk haplotypes are detected or stratifying patients as having a high probability to develop IBD if a decreased number of protective haplotypes and an increased number of risk haplotypes are detected.

20. The process of claim 19, wherein IBD comprises Crohn's Disease (CD) and ulcerative colitis (UC).

21. The process of claim 19, wherein the risk haplotypes located at the:
IL23R locus are IL23R Block 2 Haplotype 1 (B2H1) and/or IL23R Block 3 Haplotype 1 (B3H1);
IL17A locus are IL17A Haplotype 2 (H2) and/or IL17A Haplotype 4 (H4); IL17RA locus is IL17RA Block 2 Haplotype 4 (B2H4);
IL17RD locus is IL17RD Block 2 Haplotype 2 (B2H2);
IL12RB1 locus is IL12RB1 Haplotype 1 (H1); and
IL12RB2 locus are IL12RB2 Haplotype 1 (H1), IL12RB2 Haplotype 3 (H3) and/or IL12RB2 Haplotype 4 (H4).

22. The process of claim 19, wherein the protective haplotypes located at the:
IL23R locus is IL23R Block 2 Haplotype 2 (B2H2), IL23R Block 3 Haplotype 2 (B3H2) and/or IL23R Block 3 Haplotype 6 (B3H6);
IL17RA locus is IL17RA Block 1 Haplotype 3 (B1H3);
IL17RD locus IL17RD Block 1 Haplotype 2 (B1H2) and/or IL17RD Block 2 Haplotype 3 (B2H3);
IL12B(p40) locus is IL12B(p40) Haplotype (H1) and/or IL12B(p40) Haplotype; and
IL12RB2 locus is IL12RB2 Haplotype 4 (H4).

23. The process of claim 19, wherein the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP) and denaturing gradient gel electrophoresis (DGGE).

24. The process of claim 19, wherein the detection of IL23R B2H1 haplotype and/or IL23R B3H1 haplotype in a pediatric non-Jewish subject indicates an increased probability for IBD.

25. The process of claim 19, wherein the detection of IL12RB2 H4 haplotype in a subject of Ashkenazi Jewish descent indicates a decreased susceptibility to IBD and wherein the detection of IL12B2H1 haplotype and/or IL12B2H3 haplotype in a subject of Ashkenazi Jewish descent indicates an increased susceptibility to IBD.

26. The process of claim 19, wherein the detection of IL17A H2 haplotype in a non-Jewish subject and the detection of IL17A_H4 haplotype in a Jewish subject indicates an increased probability for IBD.

27. The process of claim 19, wherein the presence of ten risk haplotypes presents a greater susceptibility than the presence of nine, eight, seven, six, five, four, three, two, one or none of the risk haplotypes, and the presence of nine risk haplotypes presents a greater susceptibility than the presence of eight, seven, six, five, four, three, two, one or none of the risk haplotypes, and the presence of eight risk haplotypes presents a greater susceptibility than the presence of seven, six, five, four, three, two, one or none of the risk haplotypes, and the presence of seven risk haplotypes presents a greater susceptibility than the presence of six, five, four, three, two, one or none of the risk haplotypes, and the presence of six risk haplotypes presents a greater susceptibility than the presence of five, four, three, two, one or none of the risk haplotypes, and the presence of five risk haplotypes presents a greater susceptibility than the presence of four, three, two, one or none of the risk haplotypes, and the presence of four risk haplotypes presents a greater susceptibility than the presence of three, two, one or none of the risk haplotypes, and the presence of three risk haplotypes presents a greater susceptibility than the presence of two, one or none of the risk haplotypes, and the presence of two risk haplotypes presents a greater susceptibility than the presence of one or none of the risk haplotypes, and the presence of one risk haplotype presents a greater susceptibility than the presence of none of the risk haplotypes.

28. The process of claim 19, wherein the detection of risk and/or protective haplotypes is relative to that detected in a healthy subject.

29. A method of diagnosing susceptibility of IBD in a pediatric non-Jewish subject comprising:
Providing a sample from the pediatric non-Jewish subject;
Assaying the sample for the risk haplotypes IL23R B2H1 and/or IL23R B3H1; and
Determining that the subject has an increased susceptibility to IBD if the risk haplotypes IL23R B2H1 and/or IL23R B3H1 are present or determining that the subject has a decreased susceptibility to IBD if the risk haplotypes IL23R B2H1 and/or IL23R B3H1 are absent.

30. The method of claim 29, wherein IBD comprises Crohn's Disease (CD) and ulcerative colitis (UC).

31. The method of claim 29, wherein the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP) and denaturing gradient gel electrophoresis (DGGE).

32. The method of claim 29, wherein the detection of risk haplotypes is relative to that detected in a healthy subject.

33. The method of claim 29, wherein there is a greater susceptibility to IBD when an increased number of risk haplotypes are present and a decreased susceptibility when a decreased number of risk haplotypes are present.

34. A method of diagnosing susceptibility of IBD in subject of Ashkenazi Jewish descent comprising:
Providing a sample from the subject of Ashkenazi Jewish descent;
Assaying the sample for the risk haplotypes IL12B2H1 and/or IL12B2H3; and
Determining that the subject has an increased susceptibility to IBD if the risk haplotypes IL and/or IL12B2H3 are present or determining that the subject has a decreased susceptibility to IBD if the risk haplotypes IL12B2H1 and/or IL12B2H3 are absent.

35. The method of claim 34, wherein IBD comprises Crohn's Disease (CD) and ulcerative colitis (UC).

36. The method of claim 34, wherein the detection of risk and/or protective haplotypes comprises using a technique selected from the group consisting of allelic discrimination assay, allele-specific oligonucleotide hybridization assay, heteroplex mobility assay (HMA), single strand conformational polymorphism (SSCP), denaturing gradient gel electrophoresis (DGGE).

37. The method of claim 34, wherein the detection of risk haplotypes is relative to that detected in a healthy subject.

38. The method of claim 34, wherein there is a greater susceptibility to IBD when an increased number of risk haplotypes are present and a decreased susceptibility when a decreased number of risk haplotypes are present.


SEQUENCE LISTING (62 sequences)

SEQ ID NO: 1 - DNA, 2826 bp, Homo sapiens
acaagggtggcagcctggctctgaagtggaattatgtgcttcaaacaggttgaaagagggaaacagtcttttcctgcttccagacatgaatcaggtcactattcaatgggatgcagtaatagccctttacatactcttcagctggtgtcatggaggaattacaaatataaactgctctggccacatctgggtagaaccagccacaatttttaagatgggtatgaatatctctatatattgccaagcagcaattaagaactgccaaccaaggaaacttcatttttataaaaatggcatcaaagaaagatttcaaatcacaaggattaataaaacaacagctcggctttggtataaaaactttctggaaccacatgcttctatgtactgcactgctgaatgtcccaaacattttcaagagacactgatatgtggaaaagacatttcttctggatatccgccagatattcctgatgaagtaacctgtgtcatttatgaatattcaggcaacatgacttgcacctggaatgctgggaagctcacctacatagacacaaaatacgtggtacatgtgaagagtttagagacagaagaagagcaacagtatctcacctcaagctatattaacatctccactgattcattacaaggtggcaagaagtacttggtttgggtccaagcagcaaacgcactaggcatggaagagtcaaaacaactgcaaattcacctggatgatatagtgataccttctgcagccgtcatttccagggctgagactataaatgctacagtgcccaagaccataatttattgggatagtcaaacaacaattgaaaaggtttcctgtgaaatgagatacaaggctacaacaaaccaaacttggaatgttaaagaatttgacaccaattttacatatgtgcaacagtcagaattctacttggagccaaacattaagtacgtatttcaagtgagatgtcaagaaacaggcaaaaggtactggcagccttggagttcactgttttttcataaaacacctgaaacagttccccaggtcacatcaaaagcattccaacatgacacatggaattctgggctaacagttgcttccatctctacagggcaccttacttctgacaacagaggagacattggacttttattgggaatgatcgtctttgctgttatgttgtcaattctttctttgattgggatatttaacagatcattccgaactgggattaaaagaaggatcttattgttaataccaaagtggctttatgaagatattcctaatatgaaaaacagcaatgttgtgaaaatgctacaggaaaatagtgaacttatgaataataattccagtgagcaggtcctatatgttgatcccatgattacagagataaaagaaatcttcatcccagaacacaagcctacagactacaagaaggagaatacaggacccctggagacaagagactacccgcaaaactcgctattcgacaatactacagttgtatatattcctgatctcaacactggatataaaccccaaatttcaaattttctgcctgagggaagccatctcagcaataataatgaaattacttccttaacacttaaaccaccagttgattccttagactcaggaaataatcccaggttacaaaagcatcctaattttgctttttctgtttcaagtgtgaattcactaagcaacacaatatttcttggagaattaagcctcatattaaatcaaggagaatgcagttctcctgacatacaaaactcagtagaggaggaaaccaccatgcttttggaaaatgattcacccagtgaaactattccagaacagaccctgcttcctgatgaatttgtctcctgtttggggatcgtgaatgaggagttgccatctattaatacttattttccacaaaatattttggaaagccacttcaataggatttcactcttggaaaagtagagctgtgtggtcaaaatcaatatgagaaagctgccttgcaatctgaacttgggttttccctgcaatagaaattgaattctgcctctttttgaaaaaaatgtattcacatacaaatcttcacatggacacatgttttcatttcccttggataaatacctaggtaggggattgctgggccatatgataagcatatgtttcagttctaccaatcttgtttccagagtagtgacatttctgtgctcctaccatcaccatgtaagaattcccgggagctccatgcctttttaattttagccattcttctgcctcatttcttaaaattagagaattaaggtcccgaaggtggaacatgcttcatggtcacacatacaggcacaaaaacagcattatgtggacgcctcatgtattttttatagagtcaactatttcctctttattttccctcattgaaagatgcaaaacagctctctattgtgtacagaaagggtaaataatgcaaaatacctggtagtaaaataaatgctgaaaattttcctttaaaatagaatcattaggccaggcgtggtggctcatgcttgtaatcccagcactttggtaggctgaggtaggtggatcacctgaggtcaggagttcgagtccagcctggccaatatgctgaaaccctgtctctactaaaattacaaaaattagccggccatggtggcaggtgcttgtaatcccagctacttgggaggctgaggcaggagaatcacttgaaccaggaaggcagaggttgcactgagctgagattgtgccactgcactccagcctgggcaacaagagcaaaactctgtctggaaaaaaaaaaaaaa

SEQ ID NO: 2 - protein, 629 aa, Homo sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 3 - DNA, 1859 bp, Homo sapiens
gcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaacgaactttaagtatgagaaaagttcagcccaagtaaaataaaaactcaatcacattcaattccagagtagtttcaagtttcacatcgtaaccattttcgccc

SEQ ID NO: 4 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 5 - DNA, 3429 bp, Homo sapiens
ctgggcccgggctggaagccggaagcgagcaaagtggagccgactcgaactccaccgcggaaaagaaagcctcagaacgttcgttcgctgcgtccccagccggggccgagccctccgcgacgccagccgggccatgggggccgcacgcagcccgccgtccgctgtcccggggcccctgctggggctgctcctgctgctcctgggcgtgctggccccgggtggcgcctccctgcgactcctggaccaccgggcgctggtctgctcccagccggggctaaactgcacggtcaagaatagtacctgcctggatgacagctggattcaccctcgaaacctgaccccctcctccccaaaggacctgcagatccagctgcactttgcccacacccaacaaggagacctgttccccgtggctcacatcgaatggacactgcagacagacgccagcatcctgtacctcgagggtgcagagttatctgtcctgcagctgaacaccaatgaacgtttgtgcgtcaggtttgagtttctgtccaaactgaggcatcaccacaggcggtggcgttttaccttcagccactttgtggttgaccctgaccaggaatatgaggtgaccgttcaccacctgcccaagcccatccctgatggggacccaaaccaccagtccaagaatttccttgtgcctgactgtgagcacgccaggatgaaggtaaccacgccatgcatgagctcaggcagcctgtgggaccccaacatcaccgtggagaccctggaggcccaccagctgcgtgtgagcttcaccctgtggaacgaatctacccattaccagatcctgctgaccagttttccgcacatggagaaccacagttgctttgagcacatgcaccacatacctgcgcccagaccagaagagttccaccagcgatccaacgtcacactcactctacgcaaccttaaagggtgctgtcgccaccaagtgcagatccagcccttcttcagcagctgcctcaatgactgcctcagacactccgcgactgtttcctgcccagaaatgccagacactccagaaccaattccggactacatgcccctgtgggtgtactggttcatcacgggcatctccatcctgctggtgggctccgtcatcctgctcatcgtctgcatgacctggaggctagctgggcctggaagtgaaaaatacagtgatgacaccaaatacaccgatggcctgcctgcggctgacctgatccccccaccgctgaagcccaggaaggtctggatcatctactcagccgaccaccccctctacgtggacgtggtcctgaaattcgcccagttcctgctcaccgcctgcggcacggaagtggccctggacctgctggaagagcaggccatctcggaggcaggagtcatgacctgggtgggccgtcagaagcaggagatggtggagagcaactctaagatcatcgtcctgtgctcccgcggcacgcgcgccaagtggcaggcgctcctgggccggggggcgcctgtgcggctgcgctgcgaccacggaaagcccgtgggggacctgttcactgcagccatgaacatgatcctcccggacttcaagaggccagcctgcttcggcacctacgtagtctgctacttcagcgaggtcagctgtgacggcgacgtccccgacctgttcggcgcggcgccgcggtacccgctcatggacaggttcgaggaggtgtacttccgcatccaggacctggagatgttccagccgggccgcatgcaccgcgtaggggagctgtcgggggacaactacctgcggagcccgggcggcaggcagctccgcgccgccctggacaggttccgggactggcaggtccgctgtcccgactggttcgaatgtgagaacctctactcagcagatgaccaggatgccccgtccctggacgaagaggtgtttgaggagccactgctgcctccgggaaccggcatcgtgaagcgggcgcccctggtgcgcgagcctggctcccaggcctgcctggccatagacccgctggtcggggaggaaggaggagcagcagtggcaaagctggaacctcacctgcagccccggggtcagccagcgccgcagcccctccacaccctggtgctcgccgcagaggagggggccctggtggccgcggtggagcctgggcccctggctgacggtgccgcagtccggctggcactggcgggggagggcgaggcctgcccgctgctgggcagcccgggcgctgggcgaaatagcgtcctcttcctccccgtggaccccgaggactcgccccttggcagcagcacccccatggcgtctcctgacctccttccagaggacgtgagggagcacctcgaaggcttgatgctctcgctcttcgagcagagtctgagctgccaggcccaggggggctgcagtagacccgccatggtcctcacagacccacacacgccctacgaggaggagcagcggcagtcagtgcagtctgaccagggctacatctccaggagctccccgcagccccccgagggactcacggaaatggaggaagaggaggaagaggagcaggacccagggaagccggccctgccactctctcccgaggacctggagagcctgaggagcctccagcggcagctgcttttccgccagctgcagaagaactcgggctgggacacgatggggtcagagtcagaggggcccagtgcatgagggcggctccccagggaccgcccagatcccagctttgagagaggagtgtgtgtgcacgtattcatctgtgtgtacatgtctgcatgtgtatatgttcgtgtgtgaaatgtaggctttaaaatgtaaatgtctggattttaatcccaggcatccctcctaacttttctttgtgcagcggtctggttatcgtctatccccaggggaatccacacagcccgctcccaggagctaatggtagagcgtccttgaggctccattattcgttcattcagcatttattgtgcacctactatgtggcgggcatttgggataccaagataaattgcatgcggcatggccccagccatgaaggaacttaaccgctagtgccgaggacacgttaaacgaacaggatgggccgggcacggtggctcacgcctgtaatcccagcacactgggaggccgaggcaggtggatcactctgaggtcaggagtttgagccagcctggccaacatggtgaaaccccatctccactaaaaatagaaaaattagccgggcatggtgacacatgcctgtagtcctagctacttgggaggctgaggcaggagaattgcttgaatctgggaggcagaggttgcagtgagccgagattgtgccattgcactgcagcctggatgacagagcgagactctatctcaaaaaaaaaaaaaaaaaa

SEQ ID NO: 6 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 7 - DNA, 2347 bp, Homo sapiens
ctgtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaattttatttgtactgacaacttttcaagcaaggctgcaagtacatcagttttatgacaatcaggaagaatgcagtgttctgataccagtgccatcatacacttgtgatggatgggaacgcaagagatacttacatggaaacctgacaatgcaaacctgttgagaagatccaggagaacaagatgctagttcccatgtctgtgaagacttcctggagatggtgttgataaagcaatttagggccacttacacttctaagcaagtttaatctttggatgcctgaattttaaaagggctagaaaaaaatgattgaccagcctgggaaacataacaagaccccgtctctacaaaaaaaatttaaaattagccaggcgtggtggctcatgcttgtggtcccagctgttcaggaggatgaggcaggaggatctcttgagcccaggaggtcaaggctatggtgagccgtgattgtgccactgcataccagcctaggtgacagaatgagaccctgtctcaaaaaaaaaaatgattgaaattaaaattcagctttagcttccatggcagtcctcacccccacctctctaaaagacacaggaggatgacacagaaacaccgtaagtgtctggaaggcaaaaagatcttaagattcaagagagaggacaagtagttatggctaaggacatgaaattgtcagaatggcaggtggcttcttaacagccctgtgagaagcagacagatgcaaagaaaatctggaatccctttctcattagcatgaatgaacctgatacacaattatgaccagaaaatatggctccatgaaggtgctacttttaagtaatgtatgtgcgctctgtaaagtgattacatttgtttcctgtttgtttatttatttatttatttttgcattctgaggctgaactaataaaaactcttctttgtaatc

SEQ ID NO: 8 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 9 - DNA, 692 bp, Homo sapiens
ctggggctctgaaattgcttaggaccattttaagcaccctcaaggccataaatttctcacctcctcctgtcacccacctccacctctgagttcggcttggccactgttatagcagcacaagcattctaggacccttttggcaaaagaattattctgaggagaaagtaaaaatctgtttagtcttatgagaaatgcagatagcayagtaagaatcacagcataaagcaggtcagtgcaatccagatttaagtctttaagtttgaatgagttcatatttttgcaaactggcatttattatgtaatacatacttgaatatttagtttgttacacaagactcagatgttgaatttttattcttactgattaggtttcacatatttccaccagatcttacattttaaaaaagtatcgggaggtcgaggcgggccgatcatgaggtcaggagatcgagaccatcctggctaacatggtgaaaccccatctctactaaaaatacaaaaaattagcggggcgaggtggctggcgcctgtagtcccagctactcgggaggctgaggcaggagaatggcgtgaacccggaaggcggagcttgcagtgagccgagatcgcaccactgcactccagcctgggcgacagagcgagactccgtctcaaaaaaagaaaaaaaaaagtatcaattttattatagatgt

SEQ ID NO: 10 - DNA, 705 bp, Homo sapiens
ttgtttgcatacacttaaatgggatccacgttctgcatcatttgattgataatcaagtgaagatcctgctgaattccttttgcatatgcagaatttagattaaatttcaaaacaacacaaatacaattctcaagtcytagattctgaattaatggggttttatcctaataagacacctggggtccttgtatagtatcacagtcatagaatgatattaaagaatactgagtttcttaggctgggtgcagtggctcatgcctgtaatcccagcactttgggaggccaaggcaggcggatcacctgagctcagggattgaagaccagactggccatcatggcaaaaccccgtctctactgaaaatacaaaaaatttagccaagcctggtggtgtgtgcctgtaatcccagctactcagaaggctgaggcaagagaatcgcttgaatctgggaggtggaggttgcaatgagccaagatggagccactgcactccagcctgggtgacagagtgactctgtctccagaggaaaaaaaaaaaaaggataccaaatcctcttacttcatgcaaataggagtatgtaatagactagaaaaagtgtttagaaaatagaaaggaattatattatcagtgtctctgaataagttttcagaagccaactgttttctggttgaaactcttattctctgctccccctggtggtgctacat

SEQ ID NO: 11 - DNA, 1077 bp, Homo sapiens
cagctaccatttctccaccccattaaaagagtatattccaaaattaagaatatattccaaaattaagaatatattccaaaattaaggctgggtatggtggctcactcctgtaatctcaacactttgggaggccaaggcagagagatgacttgtgcccaggagaccagcctgggcaatataatgagaacttatctctacagaaaaatttaaaaattatccaatcatggtagtgcatgcctgtagtcccagctacttgggaggctgaggcaggaggatcacttcagcccaggaggaggtggaggttgcagtgagctgtgatcgagccactgcactccacagtccagcctgggcaacagagtgggaccctatctagaaaaaaaataaaataaaaaatatatatatacacacacacacatataaataaataaatatatatacacacataaataaatatatatacacatatatataatatcacatttggactttctggagatttgagacagttgtcaaacataaagcagtatgggctgggcacggtggctcacacctgtaatcccagcactttgggaggccaaggtgggcggatcacttgaggtcaaaaattcaagaccagcctggccaacatgatgataccccatctttactaaaaatacaaaaaagtagccaggtgttgtagtgcatgactgtaatcccagttacttgggaggctgaggcagaagaatcgcttgaacccgggaggcggaggttgcagtgaactgagatcgagccaccgcactccagcctgggcaatagagcgagactccatctcaaaaaaagcagtgtgtgtttcagttttaatgtatttcagagacagtatttgattatgtacggccaygttttatataaagaacactttgttttcctagagtctagaagacagcttggaacataataggtgttccatacatttctgctaaataaaatagttgttttaaaagcacaccacattttattattgttacccatccattttaggttaaagaatttgacaccaattttacatatgtgcaacagtcagaattctacttggagcca

SEQ ID NO: 12 - DNA, 997 bp, Homo sapiens
catttctgctaaataaaatagttgttttaaaagcacaccacattttattattgttacccatccattttaggttaaagaatttgacaccaattttacatatgtgcaacagtcagaattctacttggagccaaacattaagtacgtatttcaagtgagatgtcaagaaacaggcaaaaggtactggcagccttggagttcacygttttttcataaaacacctgaaacaggtgagtgtacttatatattttattctgttgggcttttctttatatatcttttctgctgagcacagtggctcacacctataattccagcactttgagaggccaaggcaggaagattgcttgagcctaggagtttgagactggcctgggcaacatagtgagaccctagtctgtacagaaaaataataattattattagcctgggtggtagaatgcatttgtagtcgcagctacttgggaagctgaggtagtaggattgcgtgagcccgggagtttgatgctgcagtgagctatgatcatcccactgctctctagcctggaggaaagaccaagaccctgtttcctaaaaagtttaaaacagccaggtgcagtggcttatgtctgtaatcccagcactttgggaggccaaggtgggtggattaccttaggtcaggacttcaagacctcctcggccgacatggtgaaaccctgtctctactaaaaatacgaaaattagctgggcatggtggcaggtgcctgtaatctcagctactcggaaggctgaggcaggaaaattgcttgaacccaagaagtggaggttgcagtgaactgagattgtaccaccgcactccagcctggccaagagagagagacttggtctcaaaaaaaaataaaaataaaaataataataataaataagttaaaaacaaaataaagctacaagatattttttttctctttacctttgaccaaaattgacaaaactattctagggcagatgataacatttaaat

SEQ ID NO: 13 - DNA, 720 bp, Homo sapiens
ccagtgtgaaaatactgtgcattttccccaccatccctcagcaatttcattctttaatttcagggaagcagaggagcaacttacttaagtattctaagtataggactacaaatgttcttctttaaacataaaagtcttggcgaggtgtggtggctcatgcctgtaaccccagcactttaagaggccaaggcgagtggatcacctaaggtcaggagtttaagaccaccctggccaacatggtgaaaccccgtctctactaaaaatacaaaaattaactgggtgtggtggcaggtgcctgtaatcccagctactagggaggctgaggcaggagaatctcttgaacttgagaggcggaggttgcagtgagccaagatcctgccactgcactccagcctgggtgacagagcgagactctgtctctaaataaataaataaataaagtaaaataaagataaaagtcttaagcttcaggtagaaggaaataggaacaccacagtttaaatttaaggtctgtttcctraggagaaaaatcacttaagagacaaaaataccaattaaaattaagtatccctgaaaacttggatttattaaagtttaacatgttagctaagagaaaccatagactgttctcttggtacaaattcccttctaagacacattacatgagaaacagtaaaagtgtgttagggaaagtgctcatgttaaatctctttgaaaatg

SEQ ID NO: 14 - DNA, 1001 bp, Homo sapiens
cccatacacatgttggtaatcagaggtcacagaagtgacctgtgttgtgaaagtactatatagcaagagaaattgagtatgttctttctactcagttaccttataaggcaaaagggaattgagaggaagtggctatcctagattacatgggtggatctggtaaaatcacaagagttcttataagcagaagggagaaggttgagagtcagagaaagagattggaagatgctatgcttctggctttgaaaatgaaggatggagccatgagctgaggaatgtaggcagcctctagaatatagaaaaagcaatgaaactgattctgtcctgtagcctccagaaggaacataaccctattgacaccctgatttcagcccagtggttgtgattttggatttctcacctccagaactataagataataaattcatgttgttttaagctttcaagtttgtgatgatttgtgacagtagtaataggaaactaatatagaagatgatgacytcaagaaaaagcataatcataggccaggcatggtggctcctgcctgtaagcccagcactttgggaggccaaggtgggcagagttcttgagtccaggagttcaagaccaacttggcaaacatggtgaaaccctgtctctacaaaaaaaaaaaaaaaggaagaaaaaaaattagctgggtatggtggtgcatgcctgtagttccaggtacttgaaaggccaaggtgagaggattgtttgagcccagatcttatgagctgagatcacaccactgcactccagcctgggtgacagagagagaccctgtctaaaaaagaagggaggaaggaaggaaggaaggaaggaaggaaggaaggaaggaaggaaggaaaaagaaagacagaaagaaagaaggaaagaaagaaagaaagagagagaaagaaagaaagaaagaaagaaaagaaagaaagagagagacagagaaagaaagaaagaaagagaaagaaagaaaagaaagaaaggaa

SEQ ID NO: 15 - DNA, 701 bp, Homo sapiens
aggtgcggtgcctcacacctgtaatcccagcattttgggaggctgaagcaggtggatcacctgaggtcaggagttcgagaccagtctgaccaatatggtgaaatcctgtctctactaaaaattccaaaaaaaaaaaaaaaaaaaaaaaagccacgcgtggtggcatgctcctgtaatcccagctacttgggaggttgagacaggagaattgctagaacccaggaggcagaagttgcagtgagccaggatcatgccactgcactccagcctgggcaacagagggagattctgtcttaaaaaaaaaaatccggttttgattatgtcttcatagcagtgtgaaaacagactagtacggttgatgtagaaagaagagctgaggtgatgatttggcatcatccttaaaatacagatggaatacgttattgctaaaaccaggtccttttgagtggatttgattaaactagcctggtgttttggtaggccaaaaaatatagttgttaygctttaaattttgtccaacaataagaaaccatatttctcgtttgagatcactctaaattcccacaggcacattgtcttcttgtaagactaaagtttggtgccagtgtgtacaagttatataaaaattcttcccaaattaaagataatttggattttttttagtatattcaagtatgtcctgtgagattaataggcataag

SEQ ID NO: 16 - DNA, 886 bp, Homo sapiens
tatttgaagcaactaattgggggtactggctgccacacacccttgggcattaattagtgcctggaagaggatagacagccctcaggtcaacacagtgctcggcaaaggggtctaagcagtagagcagaatgaccaagagcgtggcctgatatacctgggtttgaattaaactctgcctcttatcagctctgtgaccttggggcataattatgaacttgctgagtctcaggttttctcttttggaaaatagagataataatacttatctaacagagctgccatgagttcctaacctccactgatcccacagaaatatcaaggtgtaggtaggtctgtgtaggcatctataattagggaactgtactgaacctaagcacttggcttgyaattgattgataattcagagtgcccttacctttcttcatgtttctttttctttttcttctttttcctcttttttttttttttcctgagacagggtcttgctctgttgcccaggcgggaatgcagtggagctcactgcagcctctatctctggtgctcagttgatcctcccacctcagcctcccaagtagctgggactacaggtacatgacaccacacccatctaatttttgtattttttgtaaaaatggggttttgccatgttgtgcaggctggtctcaaactcctggactcaagcaatctgcctgccttggcctcccaaagtgctgggattacaaaatgtgagccaccatgcctacccacttcatgtttctttacgacacttcaccaccacctgacttttcttcttgttttgtttgctgtttttctgccctgtctggctagaatagaagctccatgaagacaggggctttgctcattgttttcactgctgattccccag

SEQ ID NO: 17 - DNA, 601 bp, Homo sapiens
ttctcaaacaaaaagttgtttcctggggtagttgtgcactctggaaaaacagtcactctgtggcctaaagtaaaggttaattttgcttccccccaccctttctcctttgagacctttgctttgagcagagtaaagagaatagtaattctggtatcaaatgaagactaatgcttggttaaaattatttttctttcctttcattagacaacagaggagacattggacttttattgggaatgatcgtctttgctgttatgttgtcaattctttctttgattgggatatttaacagatcattccraactgggtaggtttttgcagaatttctgttttctgatttagactacatgtatatgtatcaccaaaatttagtcatttcagttgtttactagaaaaatctgttaacatttttattcagataaaggaaaataaaaagaacaatgtttaataagtacttacccatgccaaactctctacaaatgtctttcctttaatcctcaaaatgaccctgccagaaaagcttcctggcctattttacaggtgacttaaatgaggcttaaagaggctaagtcctcagcccagaatcactgaacagtaagcc

SEQ ID NO: 18 - DNA, 601 bp, Homo sapiens
tttgaacattaaaatatttcaagggacttcataatcaagtatattttaaaacagcctcaaataaaattccgtattagtttgccttccttacaagggtattaggaatatgtttattaatgtgtaatttaaattttgaaatattaagttctgagcaaaaaacctatgtagataagaaatcattagtagactttataatagctcatttaaaatctttctactgcacttgattataaatgtaaacgaaagaaagattatttcatgaagcaaatgatggcaagaaggagaaactcagtgccaattyggcaaagaacattcaagtcaaaatttgtgagcaactggacacactggggaactgccacaccaaacaactctaatctatcgagcagcttagaaatactcaatgcatcagtaaaatttagaaatccaagggtcttgcttttctcaaagtctcattttaaataactaaccatagatctttactaataccatcacaggagggaaaaaactgaagggggccaagagtaagggactttggggctgaatgctaaaacactaaaacaattggtaaggaattgacaaatttaaaaattgtcacacttcc

SEQ ID NO: 19 - DNA, 601 bp, Homo sapiens
ttgagtagtttccggaattgtctccacaacacctggccaaggaatctgtgaggaaaagaaagatcaaatggaaaatcaaggtacatgacaccagaagacctacatgttacttcaaactttttcttcctcatgaaccattaaaatagagcataactcttctggcagctgtacatatgttcataaatacatgatattgacccatagcatagcagctctgctcagcttctaacaagtaagaatgaaaagaggacatggtctttaggaacatgaatttctgcccttcccattttccttcagaagragagattcttctatgacctcattgggggcggaaattttaaccaaaatggtgtcacccctgaacccactgcgacacgccacgtaagtgaccacagaaggagaaaagccctataaaaagagagacgatagcgctacattttgtccatctcatagcaggcacaaactcatccatccccagttgattggaagaaacaacgatgactcctgggaagacctcattggtggtgagtcctgcactaacgtgcgatgctcttgctgatttggaccagatagtatttctggaccgtgggcatgaaacgct

SEQ ID NO: 20 - DNA, 801 bp, Homo sapiens
aatagagcataactcttctggcagctgtacatatgttcataaatacatgatattgacccatagcatagcagctctgctcagcttctaacaagtaagaatgaaaagaggacatggtctttaggaacatgaatttctgcccttcccattttccttcagaaggagagattcttctatgacctcattgggggcggaaattttaaccaaaatggtgtcacccctgaacccactgcgacacgccacgtaagtgaccacagaaggagaaaagccctataaaaagagagacgatagcgctacattttgtccatctcatagcaggcacaaactcatccatccccagttgattggaagaaacaacgatgactcctgggaagacctcattggtggtgagtcctgcactaacrtgcgatgctcttgctgatttggaccagatagtatttctggaccgtgggcatgaaacgctgggttctgactatggagatccaggaatactgtatatgtaggataggaaatgaaagctttggtaggtatttaagtcattgtgcagcattttcaagaactgatacacagcagtttgaaagataagattaaaactgaaagatagctatattggggctaaaccacacaagaagtgtcacatgatgctgtgcagtaagaaagaaaatttattgaaagtctgtttttctgagtacaaaggatttaatataattctcccacggcatttttctttaaaatgggtcactatccttgagattttgaaagccgtagcagcaacaacctttgtttccattatctcgtaccata

SEQ ID NO: 21 - DNA, 511 bp, Homo sapiens
ccatggctttaaaatttttttaaaaaaactagtttcaacattctccttttgacttaggaaagacatgttatccattggttggcaataattttaataaaaatgtcaagtcatggcatgtcattagcctatcagcacatgcatcattgtcaggtctgggaaggaataataaccttgattttctaggtagaaatatcctcctgcaccattgttctcagtcccatattctgtgaaactcatcgtgaagtcaaacattcamattggaagaaagagctatagaaaatctatgtggtatcaatattcatgctagaagtgctgttggtgctactggcaggcatccaactaaaaactcgatctccttcatgttttcttaggtatattttccagttgttctaaatttaacatgtattgattctgtaataaaatcagatttcaaaaaagatacttgaagttaaatatttaaaaaatataaaccccacttattctaaaacacagttatacctatgtttagtta

SEQ ID NO: 22 - DNA, 511 bp, Homo sapiens
ggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttrgccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagc

SEQ ID NO: 23 - DNA, 1293 bp, Homo sapiens
gaacctgggtagtatggtattggtggggaggtgggggttccttggagaacttttggaagtgagaatatagtatttggtgatatgtggatgttaggaatgagggagaggcagaaggaaaaagattcagggaagccacatagatttctagcttggatgactaggtacatggtagtgctaactggggaaaatgaagagagaataaaagcaaagtgtatcaggggaggagtacatggaaagcaactgcctcttcccatccgcataccccccacccaaaatctagtgggaaataatggttcaggaccacacacacacacacacacacacatatagacatatacatcctttacaactccctctcccaacaaaaacaaaaacaattttttcttttcatcatcaccgttcagagaaagcttgaaaacgagcagcaggtttttagtgagaagcttgaaagcgtaaaggctgtgaggaactgtccctggaagctgcctggggatttcctgtaggaaaatggtgacagggatggtcacaggaatcaagatgtgagcacaaaatgactgagaggaggtggctggagaggccaacccctggatttggaatagggaaagaagcctagaaaagccatgggcctctgggtgggctggagcacactggatggagcaggatggagtgaagaggaaggtctttcaagaagcagggagcctgcagagtggcctgagaatatctagaggccttcagaagtagggcaagacagcacatgggccatgggggcgaaaatggttacgatgtgaaacttgaaactactctggaattgaatgtgattgagtttttattttacttgggctgaacttttctcatacttaaagttcrttctgccccatcagctcctttctgggttgtgtggtgccttgatcagacagaagccaggccctaggagtgttgcttgaggaagagaaaaatgttggtctgttgatctctgaggggccttaatctccaaaggaagcctgagtctaggggagaaactggacattgtagtctgaagacaatgtctcctcccagaacttcttgtattttggggagggtttcattttccccatatgatctttaataatgacatgccattcctcagggccattatcttatttgctctattcctatcaaaatgcttctgtctacagcattggctaatagtatgaaaaccttagtcggtgttcagtcttgaaggcatgtgaaatcgagaacttggaattttgggtatttccaggtcattgttactcaaagacatgctttgttttg

SEQ ID NO: 24 - DNA, 401 bp, Homo sapiens
aatctccaggccaccagaaactgctgctttcagccctctcagagcacagccaaacttccccctcagtcccagtgggggactcagtctccagtaagtacatccttcctgctacctatgtctcagtttcccaaattctagaaagcacagagaattgctcacaaaggaatccaaagccaaggcctgacgggcttttatcttaamggaacatgcgtatgagccttctggtgacagcaattagagcagccaccttgaagcaatgtgacacagtcccacctttggccgctgagtgattgcagacactcattttgcttgtctgtggtggagagaggtctctggcctcctgctttgaggctgcagccacagcttgcctggccctgtgtaagtgtttgacctatttcata

SEQ ID NO: 25 - DNA, 905 bp, Homo sapiens
tgtatatggtcaatgcgctattacctctaatagaattgttattctatatttcacctatatgtatatttttcagttttgattaaatcttttttccccttatcttccctcatgccatctcctctgcttgtgtgccctttgcccccagcaaactgtgttaacacctgttaacatgtttctattctcatttagtcatacacacactctctctctctctctctctctctctctctctctctatatatatatatatatatatatatattcagggagatctttttcatgacttatttccatagaaattgggatcatactataaaaagttatttgaaacttattttcctctctcatcaacacattccagccatctgcaggtcaacagatgtctatgcaactaattctcattctctttaatatttgcataatattccatagtagatagcaatctattcaaccrtttctagtttgatggacatttagatttaaactagatctcaccttaattcagccattctctattgatgaccattcagtaccacagaaaaagagggaaagctgtccattttttttaaagctagtataagcttaattctaaaacttgacaaatcttatacaaaaagaaaaactatggaccaatctcatttatgaacataaatccatgcaattctaaataaaatattagcaaatgtaatctagcagaatatcaaaagaacaatgcaccataatctagtggtgcattaacaaactttgtttgttaatcagtgctggagaggtatatgatgaggttcagagctgttttgaatgaaaactataaacctaatagtaactgaaggaaaagacttatatgcaaccacaactatcagaaaccaagaccaaaaacagtattccatagggcataccaaactatt

SEQ ID NO: 26 - DNA, 601 bp, Homo sapiens
tggggagcgtgcacaggtggagagtgtggtgtggctggagtggggagcgtgcacaggtggagagagtggtgtggctgggagtggggagcgtgcacaggtggagagagtggtgtggctgggagtggggagcgtgcacaggtggagagtgtggtgtggacgggagtggggagcgtgcacagtctggcattcttgctggtggacaggggaaagcttgtcctctctgtggcaccaagcaccactaccagtcaggattccttgcctggtaaggcactgcccctgcctttctcctgtctggttctcycaccctcacctgggcaggggttcgctgacccgcccttgctggagggagatgatggtcacctggagatcgtggtgtagccagccaggatcccctcctctcacattgccgctgctggctggaaggcatgggcgctctacagttctggagcccttttcctgccctctctgcccgcagatccagcccttcttcagcagctgcctcaatgactgcctcagacactccgcgactgtttcctgcccagaaatgccagacactccaggtaggggacatgcggctgtcctaggccatactgggagaaca

SEQ ID NO: 27 - DNA, 801 bp, Homo sapiens
gcactgcccctgcctttctcctgtctggttctcccaccctcacctgggcaggggttcgctgacccgcccttgctggagggagatgatggtcacctggagatcgtggtgtagccagccaggatcccctcctctcacattgccgctgctggctggaaggcatgggcgctctacagttctggagcccttttcctgccctctctgcccgcagatccagcccttcttcagcagctgcctcaatgactgcctcagacactccgcgactgtttcctgcccagaaatgccagacactccaggtaggggacatgcggctgtcctaggccatactgggagaacaagtggctgaaggcccccagcctgtgctgcgtccttacctggttctgaggggtgattagggaggagastttagtttaacttggagtccttcaggcctgaagtgtggagtggggctttagagtgtcactccctggggctggactcctggctgtctttcattagctatgtagccttaggcaaattacttaatctttttgattctcaacttccttgactggaaaatgaggtggtttttatcctagagccctagttctgtgccatgcactgagcgcagtgctccaacatgccgtccattttttcatcctcactcattgtgagtcacggtactatgcagtagaggatccccccaccccaaaccccaggttcctggataaggaaactgaggcacagagatgttgaataacttgtccaagatcacacagcagggacgctgttttcaaaagtcgcatgccctaatgcacgggaggc

SEQ ID NO: 28 - DNA, 801 bp, Homo sapiens
gcagtagaggatccccccaccccaaaccccaggttcctggataaggaaactgaggcacagagatgttgaataacttgtccaagatcacacagcagggacgctgttttcaaaagtcgcatgccctaatgcacgggaggctgcagccacgtgctcaccagaaggcaaggcgcaggcatggagccaggctggaaggagaacccagcctcccaaggaggaggcaaggtgtctcttcttagaccagcaactcaagtgtctcttgtagatggtttcattaagttcaacctggatctagagtgcctggtgcagggccaacatcattaaagccctcaagggacgtcagttgtgtttcttgtgatgactgggaagggttaagaatgctattttccctttttcctctgttytcattgcagaaccaattccgggtaagcttggatctctctccgacagcactgcagccctcaggggacattccccagtggccacttgagaagtccctgcctcagccaggcagacaaggctgaaccgaggccagcccggggtggggggtgagaccatggtttgtcgtggtggggccagagaggacagagcctggggctggggagcagggctgggggcctcagggtgggcagggcaggccccgccgcatcactcacgctgttctgctcaccgcagactacatgcccctgtgggtgtactggttcatcacgggcatctccatcctgctggtgggctccgtcatcctgctcatcgtctgcatgacctggaggctagctggtaagcgctggggctctggctgtcctg

SEQ ID NO: 29 - DNA, 801 bp, Homo sapiens
ccagccccacctcattagccttgtagtcacaggccagttacttaatacaccatggattcacttttctgtaaaatgtactgataatgcctccctctaagggtgtgacgaaggttaaatgagtagctgaggaaggtgcttgctggtggggattagtacataccagtgtcttctcccacctgcagccctctgctggccaagtcctaagccgggagaacacaggccttccggttggggcttcagcccttgcctgccccaccatgaccctaggctgctccttccgtcatctgggaagctgtttccacccttccctaggctcgtcaggattaggtgttaatcattattaattattatgtggtagaaagaaaaccagccaggcatgggaggacctatgggaggttccrataacattcagtagcatctcggccagtgctccacaggcggtgcagctctctaaaggtttggggctgggcggcggcggcgcttttggtttcctttctgctgttgcgcttctgttttccgaagtgtcctgcaccacagggtgaaggcaagaggagcctcgctgttatttggctgtcttgtgacagttctggggaagagctgaaagggttaggattgagattaaggttctaagtcgtttgctcagtcatctgtggatctcaatcctcccagctgtcactaaggagttaacccccgcagagcagttttttcatcacatctctgaggggaacaattgcttaagtatgtgggttccccttcctcacctcaaaaataccaggaggaaatgttgcaagcagcctggtg

SEQ ID NO: 30 - DNA, 768 bp, Homo sapiens
tcagtgctacaaaataactgtgatcccaattgatwatgtacaacgtgccaggcacgtcacatacacacactcatttaatacccattaaacaagagcaaatacagacccacctcacagaggaagaagctgcatttcagaggcactagtaactgctccaggtcatagtgctcgtagtggcagacccaggactcatgcctgtgcgaccacctagcacggcctcgctgctcagtctcggggctgcccccttacccttcaccctttgtcagggatggggcagacaccctgtgagctggtttctatttctcttcccaaagaaccactccagtgtatttcttttcctttccagggcctggaagtgaaaaatacagtgatgacaccaaatacaccggtcagtatttcctggtttgcatgtttgcttatttttaaagcagtggagggttctcctgggataagtgcgtgggtcgcctcctgtgctctaactcccaagtcccttcaggagaccccaccttagaaacccccttccagtaccccactcagaagggcccccaataacaaggcctggtgccattttttgaatcactcagacaaggaaagaaaggtaagtattttgtgaacagaggtcctccctggagcagaccaacagagcttggtgcctctttttcttttcttattaaagataggctaagacagctgggacgttgagagatcttttccacaggcagcagactgattttttcaggcagcaagggtggatagtacatctgggtt

SEQ ID NO: 31 - DNA, 829 bp, Homo sapiens
acctcgctgaagtagcagactacgtaggtgccgaagcaggctggcctcttgaagtccgggaggatcatgttcatggctgcagtgaacaggtcccccacgggctttccgtggtcgcagcgcagccgcacaggcgccccccggcccaggagcgcctgccacttggcgcgcgtgccgcgggagcacaggacgatgatcttagagttgctctccaccatctcctgcttctgacggcccacccaggtcatgactcctgcctccgagatggcctgctcttccagcaggtccagggccacttccgtgccgcaggcggtgagcaggaactgggcgaatttcaggaccacgtccacgtagagggggtggtcggctgagtagatgatccagactttcctgggcttcagcggtggggggatcaggtcagccrcaggcaggccatctaaggaaacaagaccacacatgctgaccctcaccccagggcccagggcagctctgtgcctgccagcccaggaggggcctggaccaggacacagagcttggctccctccctaagctgagaaacccaactgaggcctgttggaaaaacccagatgttactattccacctttggctgcctgaaaaaaatcagtctgctgccttgtgggaaaaagaatctctcaacgttcctagctggtctttagcctatactttaattaagaaaagaaaaagagcaccaagctctgttggtctgctccagggaggacctctgttcacaaatacttacctttctttccttgtctgagtgattcaaaaatggcacccagcttgttattggggcccttctgagtgggtact

SEQ ID NO: 32 - DNA, 852 bp, Homo sapiens
tgtttagccctcagcctctctccatgcagaggctcatcagacgaaaggtgccccaggcctcaggactgatgcgcacaaggctgtccccacccctgagctctggcgacatccccccaacccccaccccgatctctctcactgcctccctccttcccctccaggctccaccagcagctccctgacaagctcactccactcacctcccagcacttacccacaaactgcttccttgctgggactacgctttccccaaccacaatccttcasctcaggcatctcctcggggatcccccctgacctgggtgcctttcccgtgcatgctcacaaccctgggcaggcttccactccatctttctacttttttattttttttgagacagggtctcactctgctgcccaggctggattgcaatggtaccattatagctcactgcagcctctacctcctgggctcaagtgatcatctggcatcagcctcccgagtagctgggactacaggcatgtgccaccatgactgactaaaaaaaaatttaggtagagatgaggtctcactatgtcgcccaggctggtcttgaactcctgagctcaagcaatccacctgcctcgccttcccaaagtgctgggattacaagcatgagccactgcacctggcccattcagcgtttacatcccgcgtgaccatcttttttttttttttttttgagaagagtctcgctctgtcatccaggctgcagtgcaatggcacaatctcggctcactgcaacctctgcctcccagatcaacattctcctgcctcagcctcccagtagctaggactcagcatgtgttaccatgccccggctattttctatt

SEQ ID NO: 33 - DNA, 601 bp, Homo sapiens
tccaggccacataaggaaggcctgggccttctggcatgaaatccctgaaacccagttgcccaggatcatatgttgtgagaaataagaagagacattgctgttacaatgtcaccccacatcaacttttggcattctcttccaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttmgatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaatgtgaatttaattttatttgtactgacaacttttcaagcaaggctgcaagtacatcagttttatgacaatcaggaagaatgcagtgttct

SEQ ID NO: 34 - DNA, 601 bp, Homo sapiens
ggcaaggcaattgtgctagaaagatgaaagctgggccaaacgatttctccctcaagggcttacaaagtacaaaagctgcacctacatgtggagtgtctgccagtaggtggtgcaagttctatgcacacccctgtgaattgcaagcacagtgccctaagaccaagatgggcttgttttgggagagtatgcattgcagaaacaggctcagcttaccctgtgactatgttgccaaggggtcttcacagctttccttctcttttgcagaaagatagagtcttcacggacaagacctcagccacgktcatctgccgcaaaaatgccagcattagcgtgcgggcccaggaccgctactatagctcatcttggagcgaatgggcatctgtgccctgcagttaggtgagcaggccctcaaaggccagcccaggcctgcactctcagtgcacctggatgcagggatatgattgggggctgtgttggagaggaaagggggatggagtggccagcacccagttgccagaatcagaaacatacatttattcactaacagatatttatttggtgcctttgttatgtaggacactgtgctggccacagggatatt

SEQ ID NO: 35 - DNA, 601 bp, Homo sapiens
ctctggtttctcagcatttttctagaactatttcattaagaaattaagggcaacctctcagtgacctatcagttaatgataatgggaaaagcaaagtcaaacccgtgttttttcaaccgcccttccttgtctacattgaagaaagaacatggagattttagccgattgcttgaataaatgtatgtgttggggcaggatattattgggaactgagaatagtctctgctgtgtttgaacccactcatccaaattgcctggccatgcttcctgaagcctcatagcaccaaagaaagggataaamggagaattcaaagctacaaatgacttgctgaaattgcaccttgagtcaaaaataaaaacaagagctccagggcgtagatcttaggggccctgaagcagactccaaaactcgatgaggcctcccgaaattttcccagggccacctcaactccttttacttctgctgacaccactaatctgaagttcgctgttggtccaatgcacctggactttccgtaagaaagcaacttccataaatacaagacctatgtgttaacccccatgtggcttactttaatcatcaccgaagcaaaccccaggt

SEQ ID NO: 36 - DNA, 801 bp, Homo sapiens
ctgctgaatattgtgccctgccgtattctctatgaaactgaaattgtgctggaagtttctctcccccagacctttggcaaagagtcttgtgctgtttgcagtttttggtatattaaggtgtttccaatctgctaaataatcaaaggttactattaaaggcagccttccagtcaatgagtcgatggcagctataaaactctttgtttctcttttccatgaccttgagcccaagcagggtctcatgccttgagatcatctcagcaagcatttgccaaatacttgttgtaaacaaggttgtgtttaggcaatggggatgcccgaagggttaataaaacacagtcccagagttcctggagcttacagcctggttctccactttatgtgcattccagtttatgtcrtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaaggtaattctatacccttggatagtatcaattttctctttcgctcataagagttaaaaacaacaacaacaacaaattgaaaagccaagtcatggtgagtgtaatgaattaacatcaagtctcttattgatgttaa

SEQ ID NO: 37 - DNA, 1165 bp, Homo sapiens
ttgaaatgatacttaatagccctctctattcccctgttctaaactagatctttcctctttatcttttccatcctcttccacagtgcatgactccctttcccctcatcaaacctgtcattaggtccagtcagttctacactcaaaatccttcatgggttgggcgaggtggctcatgcctataatcccagcactttgggaggccaagacgggcagagtgcttgagttcaagagttggagactagcctgggtatcatgggaaaaccccatgtctacacaaaatacaaaaattaattgggcatggtagtacgtgcctgtggtcccggctactcaggaggctggggtgggaggatcactcgagcccaggaggtcaaggttgcagtgagccaagatggccccactgcacgccagcctgggtgacagagccagaccctgtctcaaaatccttcatatccacccccattggcttccacctatttcagactaataatccaattctggattattgtatagccttttgatttttccactaccaccactttcatttcccacaaatcctccgtaaaggtgctagacagtcccagagaccctcaaaatagtgtgaatctcactatatgcactcactttctatcacttacatgatacagtcctgatccatcagtagctcatggaaggtcttctgacaggcctgctccactacaagtttcttcttgcattttcaggatccaggctattgcacttgttgaaccttctcagaaatgtcatgttatttcaygtatttgtttcagaacatgctggttgtctctgtccaggaggttggccttccccctaccccattgcgatctctcaccccatcctgactcctccccaccactccatcctcaccctatgtcctgatagaaatgtctgacttcacagtccttgcactgaaccaaaatgcagtgctcttttccaaaggcagctattctggctattccaagctatgtccagtgccgacttccctaacaggcacagtaggcacagtggctagggcccacaataattttaggagtccatgaaaatgtttaattttacttaaaatcagaagagaaaaataactgttatgttcatgtatatgcatgtatatggcatgcatatgtatacatctatgtatatgtgtgtatatttt

SEQ ID NO: 38 - DNA, 511 bp, Homo sapiens
acctcaccctgtccctacctctgtatgacattgagtaagcagcaacacctctctgggccttagacacccgctgtgtgccttccctgcaggtttgaacccaccaggacctaaaagggagggcacagaggaggggtaggcgcaggccattccaggccattacccattccagccggatccgggtcttgcggcgcagtcaggctgcaggtggcaaggcccccgtcctggcccacaggctgccattcaatgcaatacgtcrtgctctgagcccgggctggccaatacatggtggtcccgttggttccgacgctgatattcagagccactggttctggaaggagaggggagagacgcatcgagacagttgccatctctctccccagtccccttctctggctcctgcagcctctctcccaccctctcgtctatacccctgaccctacagggccaggggtgtctgtgtttggctgtttccctgcactggaggctgccttggggccaggcacaaagtggg

SEQ ID NO: 39 - DNA, 643 bp, Homo sapiens
  misc_feature (18)..(18): n is a, c, g, or t
ggacacagagtgagaccntacctcgctatgttttacataaataaataaataaataaataagtaaagtatacggaaggatgtgcataggttatatgcaaatactttaccattttatatcaggaacttgagcatctttggattttggtatctgcagagggtcctggaaccattgccccgtaaatatggaaatgactgtatacattatattatgtatgtctatctattgtaaactacatacataagatactatactatgtatacctattagatacatacttgctttttattccttccttagttttttttctcattgaactccgggaggtggaaaaaatttagtgcatttcacagaagagaaaatctagaccagaggaaagatgtctaagttcgatcccagaagccatgactgcccatctcccatttgacagcaggaaagactgaggcacagagagatgaaaccaacccacacagcaggcccacagctctccacacatacacgtgcctccacccagcaagaggagccgccatgccagggtcaggggactcaccgccctgcctggacagcaggaagaggaagaggagggggaccacccaggtcaccagcggctccatcrgatccacgtagagccccacagccccaggg

SEQ ID NO: 40 - DNA, 601 bp, Homo sapiens
ggctttaagaaagaacttttttaaaagctggattcacttctgaatctttaaaataatcccccttaatcaaatctcattttcaccaaataactactaccaacattagtggtaagtttcaactacacttagtccagtggtttttcaaaaaccaatgtgccaaacccaagagttccaatgactgagaagccagagacggctagtgatgactgcctcaggaggagaagaaatgttcgtaggaaaattcatagcaaagggacagaggaaaagaacaagggggcgagaaggtagcctagagatgaayggtctatgaaagaaatgggaggaagcctgagaagaaatatggtggaaaagtgtatagtaaatgccaatgatcagactcatcgttagcaatatgtatgcagagaagtgccatggaaagagaggcgagaagcacatagtgtgaggttggaacttcaggcagcccagttctgcctggatggtcagaacttggaatgcttgagattggcctctgattcaactggaccgattacatttgctcagaaatgtactggctcactccagggatggaggtgagtattctcagagcaagatgcatctgtga

SEQ ID NO: 41 - DNA, 601 bp, Homo sapiens
accttatccagaactgctgcatcgtgactcatctttgttgaaggaaaatgctgtgcgattcgaattgagctactgattggcatgctcataagaggtttcctggtgaactgcactctttatttctcaggactcacatcctcttcactacacctcttccagttgaggatagaaagttttggaattatgaatctgaatgagcacaaaaccagaagagtaagaataacaattataaagtcctctgatggggttcacatgatgggttttgatcattgcacacttcttcagttttaagaaatgagtkatttcttcttctttctcctccctgtctctctcacacacacataaaacgtctggtataagaagagactactataaatagcctggtgaatcatgatcaggctcttcactttgaaactaacactgcagctcaaaatttattttaaaatgaatctactttcaatagcaagacaattctcaaagcaggacatttagaacagccataatttgtgaatattatagaagaacccagcctaattatttccttttcacgagcaagtgactggttaaaaccaaaattcctcctctcatgtaaaggtttctc

SEQ ID NO: 42 - DNA, 644 bp, Homo sapiens
aagatcagttacagtgcaggctaaactgccttaaaaaaaaaaagaggacccaaagcacagtggcttaaataatactttatttatcttccatataacagccctgaggtaaggtatccagactggtgggcagctgtgctccatagttatttgtggactcagttttcttccatctttttggcataccatcccttaggtattgtcctcatctgcatgatctctatgagatcacagacacttctgctcatatttctttggagaaaacctagtcacatggccacatctcttacttcaaaaggttagacaaattgtaaagcatacttttaaaaaaaaaaaaaaatcaaagcttatgaaagccaagtctttaaaaatgtttcatattgttcatgaactttaaaaaaaataagtcacatgcatttataagaaatgtttttaggatttttcctkctacatcataaaagacctgtatttcctcttattgccaaatacaatcactgttaatagatatttcagatatttccctataatacttttttcttggcatttttaacatggttgtgttcaagctatttataccattttgaatcccgtctttttttcaactgttcacataatttacctgtcattacaaactctttgtaaatatt

SEQ ID NO: 43 - DNA, 2800 bp, Homo sapiens
ttttttttttttttccagacagagttttgctcttgttgcccaggctggagtgcagtggcacaatctcagctcagtgcaacctctgccttcctggttcaagtgattctcctgcctcagcctcccaagtagctgggattacaagcacctgccaccatggccggctaatttttgtaattttagtagagacagggtttcagcatattggccaggctggactcgaactcctgacctcaggtgatccacctacctcagcctaccaaagtgctgggattacaagcatgagccaccacgcctggcctaatgattctattttaaaggaaaattttcagcatttattttactaccaggtattttgcattatttaccctttctgtacacaatagagagctgttttgcatctttcaatgagggaaaataaagaggaaatagttgactctataaaaaatacatgaggcgtccacataatgctgtttttgtgcctgtatgtgtgaccatgaagcatgttccaccttcgggaccttaattctctaattttaagaaatgaggcagaagaatggctaaaattaaaaaggcatggagctcccgggaattcttacatggtgatggtaggagcacagaaatgtcactactctggaaacaagattggtagaactgaaacatatgcttatcatatggcccagcaatcccctacctaggtatttatccaagggaaatgaaaacatgtgtccatgtgaagatttgtatgtgaatacatttttttcaaaaagaggcagaattcaatttctattgcagggaaaacccaagttcagattgcaaggcagctttctcatattgattttgaccacacagctctacttttccaagagtgaaatcctattgaagtggctttccaaaatattttgtggaaaataagtattaatagatggcaactcctcattcacgatccccaaacaggagacaaattcatcaggaagcagggtctgttctggaatagtttcactgggtgaatcattttccaaaagcatggtggtttcctcctctactgagttttgtatgtcaggagaactgcattctccttgatttaatatgaggcttaattctccaagaaatattgtgttgcttagtgaattcacacttgaaacagaaaaagcaaaattaggatgcttttgtaacctgggattatttcctgagtctaaggaatcaactggtggtttaagtgttaaggaagtaatttcattattattgctgagatggcttccctcaggcagaaaatttgaaatttggggtttatatccagtgttgagatcaggaatatatacaactgtagtattgtcgaatagcgagttttgcgggtagtctcttgtctccaggggtcctgtattctccttcttgtagtctgtaggcttgtgttctgggatgaagatttcttttatctctgtaatcatgggatcaacatataggacctgctcactggaattattattcataagttcactattttcctgtagcattttcacaacattgctgtttttcatattaggaatatcttcataaagccactttggtattaacaataagatccttcttttaatcccagttcggaatgatctgttaaatatcccaatcaaagaaagaattgacaacataacagcaaagacgatcattcccaataaaagtccaatgtctcctctgttgtcagaagtaaggtgccctgtagagatggaagcaactgttagcccagaattccatgtgtcatgttggaatgcttttgatgtgacctggggaactgtttcaggtgttttatgaaaaaacagtgaactccaaggctgccagtaccttttgcctgtttcttgacatctcacttgaaatacgtacttaatgtttggctccaagtagaattctgactgttgcacatatgtaaaattggtgtcaaattctttaacattccaagtttggtttgttgtagccttgtatctcatttcacaggaaaccttttcaattgttgtttgactatcccaataaattatggtcttgggcactgtagcatttatagtctcagccctggaaatgacggctgcagaaggtatcactatatcatccaggtgaatttgcagttgttttgactcttccatgcctagtgcgtttgctgcttggacccaaaccaagtacttcttgccaccttgtaatgaatcagtggagatgttaatatagcttgaggtgagatactgttgctcttcttctgtctctaaactcttcacatgtaccacgtattttgtgtctatgtaggtgagcttcccagcattccaggtgcaagtcatgttgcctgaatattcataaatgacacaggttacttcatcaggaatatctggcggatatccagaagaaatgtcttttccacatatcagtgtctcttgaaaatgtttgggacattcagcagtgcagtacatagaagcatgtggttccagaaagtttttataccaaagccgagctgttgttttattaatccttgtgatttgaaatctttctttgatgccatttttataaaaatgaagtttccttggttggcagttcttaattgctgcttggcaatatatagagatattcatacccatcttaaaaattgtggctggttctacccagatgtggccagagcagtttatatttgtaattcctccatgacaccagctgaagagtatgtaaagggctattactgcatcccattgaatagtgacctgattcatgtctggaagcaggaaaagactgtttccctctttcaacctgtttgaagcacataattcca

SEQ ID NO: 44 - DNA, 1859 bp, Homo sapiens
gggcgaaaatggttacgatgtgaaacttgaaactactctggaattgaatgtgattgagtttttattttacttgggctgaacttttctcatacttaaagttcgttctgccccatcagctcctttctgggttgtgtggtgccttgatcagacagaagccaggccctaggagtgttgcttgaggaagagaaaaatgttggtctgttgatctctgaggggccttaatctccaaaggaagcctgagtctaggggagaaactggacattgtagtctgaagacaatgtctcctcccagaacttcttgtattttggggagggtttcattttccccatatgatctttaataatgacatgccattcctcagggccattatcttatttgctctattcctatcaaaatgcttctgtctacagcattggctaatagtatgaaaaccttagtcggtgttcagtcttgaaggcatgtgaaatcgagaacttggaattttgggtatttccaggtcattgttactcaaagacatgctttgttttgcttataggagaatttgcataactcttcccaatagggaagagctgagtttgctaaaatacatttaatgagaacaaataaacattttcttttgaaactttattattacagaaggattttattataagggtttaatggaacagagagttagacttgctgtatctcttgtaaaacctcaacaaattgcaaaatttaaattaatacatatgaaagtaggtttaatctttcttttcagccatgttataacttttaaaaacaaagaggaggaaatactatatccgtatgttgtaaagactcagaaattttgcactgccatttaattctatcttacatttacctaggcagaactgataatagtgctttttcacctttttctaaatacttaaacataaatagatccttaaatagctcaaataaatacaggttttaccttttaaataataaaattcccaagcccagaatcttatcttctttaaaaagacaaacaaaataaagtaggtaattcattaaaatggaatcttataccccaaagttatctcagggcatattaaatacataaataacttaaagcccttaaaaaagtaaacaagaagcaaattggtactcagtcaaaccttccttcttggaaagagggaaaggtaattcagatctcatatcttggccaagtgttacctctgaagctttaattggacaaactgccttaagaactctaatgagtttagtccgaaatgaggctgtctttgaaggatgagggttcctgaggggctgggtcggctctccatagtctaactgctttggggagtgtgggctccccagagctcttaggccacatggtggacaatcggggtgacacaggtgcagcccacggacaccagtatcttctccagccggaaggagttggggcagtgtggaggctccctgcgcaggaccaggatctcttgctggatggggacagagttcatgtggtagtccacgttcccatcagcgttgatgcagcccaagtggcggcactttgcctcccagatcacagagggatatctctcagggtcctcattgcggtggagattccaaggtgaggtggatcggttgtagtaatctgaggaccttttgggattggtattggtattccggttatggatgttcaggttgaccatcacagtccgggggaagttcttgtcctcagaatttgggcatcctggatttcgtgggattgtgattcctgccttcactatggcctccaggctcagcagcagtagcagtgacaccaatgaggtcttcccaggagtcatcgttgtttcttccaatcaactggggatggatgagtttgtgcctgc

SEQ ID NO: 45 - DNA, 3429 bp, Homo sapiens
ttttttttttttttttttgagatagagtctcgctctgtcatccaggctgcagtgcaatggcacaatctcggctcactgcaacctctgcctcccagattcaagcaattctcctgcctcagcctcccaagtagctaggactacaggcatgtgtcaccatgcccggctaatttttctatttttagtggagatggggtttcaccatgttggccaggctggctcaaactcctgacctcagagtgatccacctgcctcggcctcccagtgtgctgggattacaggcgtgagccaccgtgcccggcccatcctgttcgtttaacgtgtcctcggcactagcggttaagttccttcatggctggggccatgccgcatgcaatttatcttggtatcccaaatgcccgccacatagtaggtgcacaataaatgctgaatgaacgaataatggagcctcaaggacgctctaccattagctcctgggagcgggctgtgtggattcccctggggatagacgataaccagaccgctgcacaaagaaaagttaggagggatgcctgggattaaaatccagacatttacattttaaagcctacatttcacacacgaacatatacacatgcagacatgtacacacagatgaatacgtgcacacacactcctctctcaaagctgggatctgggcggtccctggggagccgccctcatgcactgggcccctctgactctgaccccatcgtgtcccagcccgagttcttctgcagctggcggaaaagcagctgccgctggaggctcctcaggctctccaggtcctcgggagagagtggcagggccggcttccctgggtcctgctcctcttcctcctcttcctccatttccgtgagtccctcggggggctgcggggagctcctggagatgtagccctggtcagactgcactgactgccgctgctcctcctcgtagggcgtgtgtgggtctgtgaggaccatggcgggtctactgcagcccccctgggcctggcagctcagactctgctcgaagagcgagagcatcaagccttcgaggtgctccctcacgtcctctggaaggaggtcaggagacgccatgggggtgctgctgccaaggggcgagtcctcggggtccacggggaggaagaggacgctatttcgcccagcgcccgggctgcccagcagcgggcaggcctcgccctcccccgccagtgccagccggactgcggcaccgtcagccaggggcccaggctccaccgcggccaccagggccccctcctctgcggcgagcaccagggtgtggaggggctgcggcgctggctgaccccggggctgcaggtgaggttccagctttgccactgctgctcctccttcctccccgaccagcgggtctatggccaggcaggcctgggagccaggctcgcgcaccaggggcgcccgcttcacgatgccggttcccggaggcagcagtggctcctcaaacacctcttcgtccagggacggggcatcctggtcatctgctgagtagaggttctcacattcgaaccagtcgggacagcggacctgccagtcccggaacctgtccagggcggcgcggagctgcctgccgcccgggctccgcaggtagttgtcccccgacagctcccctacgcggtgcatgcggcccggctggaacatctccaggtcctggatgcggaagtacacctcctcgaacctgtccatgagcgggtaccgcggcgccgcgccgaacaggtcggggacgtcgccgtcacagctgacctcgctgaagtagcagactacgtaggtgccgaagcaggctggcctcttgaagtccgggaggatcatgttcatggctgcagtgaacaggtcccccacgggctttccgtggtcgcagcgcagccgcacaggcgccccccggcccaggagcgcctgccacttggcgcgcgtgccgcgggagcacaggacgatgatcttagagttgctctccaccatctcctgcttctgacggcccacccaggtcatgactcctgcctccgagatggcctgctcttccagcaggtccagggccacttccgtgccgcaggcggtgagcaggaactgggcgaatttcaggaccacgtccacgtagagggggtggtcggctgagtagatgatccagaccttcctgggcttcagcggtggggggatcaggtcagccgcaggcaggccatcggtgtatttggtgtcatcactgtatttttcacttccaggcccagctagcctccaggtcatgcagacgatgagcaggatgacggagcccaccagcaggatggagatgcccgtgatgaaccagtacacccacaggggcatgtagtccggaattggttctggagtgtctggcatttctgggcaggaaacagtcgcggagtgtctgaggcagtcattgaggcagctgctgaagaagggctggatctgcacttggtggcgacagcaccctttaaggttgcgtagagtgagtgtgacgttggatcgctggtggaactcttctggtctgggcgcaggtatgtggtgcatgtgctcaaagcaactgtggttctccatgtgcggaaaactggtcagcaggatctggtaatgggtagattcgttccacagggtgaagctcacacgcagctggtgggcctccagggtctccacggtgatgttggggtcccacaggctgcctgagctcatgcatggcgtggttaccttcatcctggcgtgctcacagtcaggcacaaggaaattcttggactggtggtttgggtccccatcagggatgggcttgggcaggtggtgaacggtcacctcatattcctggtcagggtcaaccacaaagtggctgaaggtaaaacgccaccgcctgtggtgatgcctcagtttggacagaaactcaaacctgacgcacaaacgttcattggtgttcagctgcaggacagataactctgcaccctcgaggtacaggatgctggcgtctgtctgcagtgtccattcgatgtgagccacggggaacaggtctccttgttgggtgtgggcaaagtgcagctggatctgcaggtcctttggggaggagggggtcaggtttcgagggtgaatccagctgtcatccaggcaggtactattcttgaccgtgcagtttagccccggctgggagcagaccagcgcccggtggtccaggagtcgcagggaggcgccacccggggccagcacgcccaggagcagcaggagcagccccagcaggggccccgggacagcggacggcgggctgcgtgcggcccccatggcccggctggcgtcgcggagggctcggccccggctggggacgcagcgaacgaacgttctgaggctttcttttccgcggtggagttcgagtcggctccactttgctcgcttccggcttccagcccgggcccag

SEQ ID NO: 46 - DNA, 2100 bp, Homo sapiens
ggtggctgaacctcgcaggtggcagagaggctcccctggggctgtggggctctacgtggatccgatggagccgctggtgacctgggtggtccccctcctcttcctcttcctgctgtccaggcagggcgctgcctgcagaaccagtgagtgctgttttcaggacccgccatatccggatgcagactcaggctcggcctcgggccctagggacctgagatgctatcggatatccagtgatcgttacgagtgctcctggcagtatgagggtcccacagctggggtcagccacttcctgcggtgttgccttagctccgggcgctgctgctacttcgccgccggctcagccaccaggctgcagttctccgaccaggctggggtgtctgtgctgtacactgtcacactctgggtggaatcctgggccaggaaccagacagagaagtctcctgaggtgaccctgcagctctacaactcagttaaatatgagcctcctctgggagacatcaaggtgtccaagttggccgggcagctgcgtatggagtgggagaccccggataaccaggttggtgctgaggtgcagttccggcaccggacacccagcagcccatggaagttgggcgactgcggacctcaggatgatgatactgagtcctgcctctgccccctggagatgaatgtggcccaggaattccagctccgacgacggcagctggggagccaaggaagttcctggagcaagtggagcagccccgtgtgcgttccccctgaaaaccccccacagcctcaggtgagattctcggtggagcagctgggccaggatgggaggaggcggctgaccctgaaagagcagccaacccagctggagcttccagaaggctgtcaagggctggcgcctggcacggaggtcacttaccgactacagctccacatgctgtcctgcccgtgtaaggccaaggccaccaggaccctgcacctggggaagatgccctatctctcgggtgctgcctacaacgtggctgtcatctcctcgaaccaatttggtcctggcctgaaccagacgtggcacattcctgccgacacccacacagaaccagtggctctgaatatcagcgtcggaaccaacgggaccaccatgtattggccagcccgggctcagagcatgacgtattgcattgaatggcagcctgtgggccaggacgggggccttgccacctgcagcctgactgcgccgcaagacccggatccggctggaatggcaacctacagctggagtcgagagtctggggcaatggggcaggaaaagtgttactacattaccatctttgcctctgcgcaccccgagaagctcaccttgtggtctacggtcctgtccacctaccactttgggggcaatgcctcagcagctgggacaccgcaccacgtctcggtgaagaatcatagcttggactctgtgtctgtggactgggcaccatccctgctgagcacctgtcccggcgtcctaaaggagtatgttgtccgctgccgagatgaagacagcaaacaggtgtcagagcatcccgtgcagcccacagagacccaagttaccctcagtggcctgcgggctggtgtagcctacacggtgcaggtgcgagcagacacagcgtggctgaggggtgtctggagccagccccagcgcttcagcatcgaagtgcaggtttctgattggctcatcttcttcgcctccctggggagcttcctgagcatccttctcgtgggcgtccttggctaccttggcctgaacagggccgcacggcacctgtgcccgccgctgcccacaccctgtgccagctccgccattgagttccctggagggaaggagacttggcagtggatcaacccagtggacttccaggaagaggcatccctgcaggaggccctggtggtagagatgtcctgggacaaaggcgagaggactgagcctctcgagaagacagagctacctgagggtgcccctgagctggccctggatacagagttgtccttggaggatggagacaggtgcaaggccaagatgtgatcgttgaggctcagagagggtgagtgactcgcccgaggctacgtagc

SEQ ID NO: 47 - DNA, 2100 bp, Homo sapiens
gctacgtagcctcgggcgagtcactcaccctctctgagcctcaacgatcacatcttggccttgcacctgtctccatcctccaaggacaactctgtatccagggccagctcaggggcaccctcaggtagctctgtcttctcgagaggctcagtcctctcgcctttgtcccaggacatctctaccaccagggcctcctgcagggatgcctcttcctggaagtccactgggttgatccactgccaagtctccttccctccagggaactcaatggcggagctggcacagggtgtgggcagcggcgggcacaggtgccgtgcggccctgttcaggccaaggtagccaaggacgcccacgagaaggatgctcaggaagctccccagggaggcgaagaagatgagccaatcagaaacctgcacttcgatgctgaagcgctggggctggctccagacacccctcagccacgctgtgtctgctcgcacctgcaccgtgtaggctacaccagcccgcaggccactgagggtaacttgggtctctgtgggctgcacgggatgctctgacacctgtttgctgtcttcatctcggcagcggacaacatactcctttaggacgccgggacaggtgctcagcagggatggtgcccagtccacagacacagagtccaagctatgattcttcaccgagacgtggtgcggtgtcccagctgctgaggcattgcccccaaagtggtaggtggacaggaccgtagaccacaaggtgagcttctcggggtgcgcagaggcaaagatggtaatgtagtaacacttttcctgccccattgccccagactctcgactccagctgtaggttgccattccagccggatccgggtcttgcggcgcagtcaggctgcaggtggcaaggcccccgtcctggcccacaggctgccattcaatgcaatacgtcatgctctgagcccgggctggccaatacatggtggtcccgttggttccgacgctgatattcagagccactggttctgtgtgggtgtcggcaggaatgtgccacgtctggttcaggccaggaccaaattggttcgaggagatgacagccacgttgtaggcagcacccgagagatagggcatcttccccaggtgcagggtcctggtggccttggccttacacgggcaggacagcatgtggagctgtagtcggtaagtgacctccgtgccaggcgccagcccttgacagccttctggaagctccagctgggttggctgctctttcagggtcagccgcctcctcccatcctggcccagctgctccaccgagaatctcacctgaggctgtggggggttttcagggggaacgcacacggggctgctccacttgctccaggaacttccttggctccccagctgccgtcgtcggagctggaattcctgggccacattcatctccagggggcagaggcaggactcagtatcatcatcctgaggtccgcagtcgcccaacttccatgggctgctgggtgtccggtgccggaactgcacctcagcaccaacctggttatccggggtctcccactccatacgcagctgcccggccaacttggacaccttgatgtctcccagaggaggctcatatttaactgagttgtagagctgcagggtcacctcaggagacttctctgtctggttcctggcccaggattccacccagagtgtgacagtgtacagcacagacaccccagcctggtcggagaactgcagcctggtggctgagccggcggcgaagtagcagcagcgcccggagctaaggcaacaccgcaggaagtggctgaccccagctgtgggaccctcatactgccaggagcactcgtaacgatcactggatatccgatagcatctcaggtccctagggcccgaggccgagcctgagtctgcatccggatatggcgggtcctgaaaacagcactcactggttctgcaggcagcgccctgcctggacagcaggaagaggaagaggagggggaccacccaggtcaccagcggctccatcggatccacgtagagccccacagccccaggggagcctctctgccacctgcgaggttcagccacc

SEQ ID NO: 48 - DNA, 1881 bp, Homo sapiens
ggtggctgaacctcgcaggtggcagagaggctcccctggggctgtggggctctacgtggatccgatggagccgctggtgacctgggtggtccccctcctcttcctcttcctgctgtccaggcagggcgctgcctgcagaaccagtgagtgctgttttcaggacccgccatatccggatgcagactcaggctcggcctcgggccctagggacctgagatgctatcggatatccagtgatcgttacgagtgctcctggcagtatgagggtcccacagctggggtcagccacttcctgcggtgttgccttagctccgggcgctgctgctacttcgccgccggctcagccaccaggctgcagttctccgaccaggctggggtgtctgtgctgtacactgtcacactctgggtggaatcctgggccaggaaccagacagagaagtctcctgaggtgaccctgcagctctacaactcagttaaatatgagcctcctctgggagacatcaaggtgtccaagttggccgggcagctgcgtatggagtgggagaccccggataaccaggttggtgctgaggtgcagttccggcaccggacacccagcagcccatggaagttgggcgactgcggacctcaggatgatgatactgagtcctgcctctgccccctggagatgaatgtggcccaggaattccagctccgacgacggcagctggggagccaaggaagttcctggagcaagtggagcagccccgtgtgcgttccccctgaaaaccccccacagcctcaggtgagattctcggtggagcagctgggccaggatgggaggaggcggctgaccctgaaagagcagccaacccagctggagcttccagaaggctgtcaagggctggcgcctggcacggaggtcacttaccgactacagctccacatgctgtcctgcccgtgtaaggccaaggccaccaggaccctgcacctggggaagatgccctatctctcgggtgctgcctacaacgtggctgtcatctcctcgaaccaatttggtcctggcctgaaccagacgtggcacattcctgccgacacccacacagatggcatgatctcagctcactgcaacctccgccttccagattcaagagattctcctgcttcagcctcccgagtagctgggattacaggcatctgccaccatacccggctaattttgtatttttagtagagacggggtttcaccacgttggccaggctggtctcgaactcctgacctcaagtgatccacctgccttggcctcccaaagtgttgggattataggcgtgagccaccatgcccagcctaatttttgtatttttagtagagatggagtttcaccatgttgcccaggctggtctcaaactcctgccctcaggtgatccacccacctcagcctctcaaagtgctgggattacaggtgtgagccactgtggccgacctactatttttattatttttgagctaggttctcagtctgttggcagactggagtgcaatcatggctcactgcagccttgaactcccagactcaagtgatccttccacctcagcctctggagtagctgggactacagacatgcaccaccacacctggttaattttttatttttattttttgtagagacaggtgtctctctacgttgcccaggctggtctcgaactcctgggctcaagtgatccacccatctccacctcccaaagtgctaggattacaggcgtgagccaccgtacccagcctggtcccatatcatagtgaaatggtgcctgtaaagctctcagcattggcttggcacatgcagttggtactcaataaacggctgttgctatccccaaaaaaaaaaaaaaaaaaaaaaaaa

SEQ ID NO: 49 - DNA, 1881 bp, Homo sapiens
tttttttttttttttttttttttttggggatagcaacagccgtttattgagtaccaactgcatgtgccaagccaatgctgagagctttacaggcaccatttcactatgatatgggaccaggctgggtacggtggctcacgcctgtaatcctagcactttgggaggtggagatgggtggatcacttgagcccaggagttcgagaccagcctgggcaacgtagagagacacctgtctctacaaaaaataaaaataaaaaattaaccaggtgtggtggtgcatgtctgtagtcccagctactccagaggctgaggtggaaggatcacttgagtctgggagttcaaggctgcagtgagccatgattgcactccagtctgccaacagactgagaacctagctcaaaaataataaaaatagtaggtcggccacagtggctcacacctgtaatcccagcactttgagaggctgaggtgggtggatcacctgagggcaggagtttgagaccagcctgggcaacatggtgaaactccatctctactaaaaatacaaaaattaggctgggcatggtggctcacgcctataatcccaacactttgggaggccaaggcaggtggatcacttgaggtcaggagttcgagaccagcctggccaacgtggtgaaaccccgtctctactaaaaatacaaaattagccgggtatggtggcagatgcctgtaatcccagctactcgggaggctgaagcaggagaatctcttgaatctggaaggcggaggttgcagtgagctgagatcatgccatctgtgtgggtgtcggcaggaatgtgccacgtctggttcaggccaggaccaaattggttcgaggagatgacagccacgttgtaggcagcacccgagagatagggcatcttccccaggtgcagggtcctggtggccttggccttacacgggcaggacagcatgtggagctgtagtcggtaagtgacctccgtgccaggcgccagcccttgacagccttctggaagctccagctgggttggctgctctttcagggtcagccgcctcctcccatcctggcccagctgctccaccgagaatctcacctgaggctgtggggggttttcagggggaacgcacacggggctgctccacttgctccaggaacttccttggctccccagctgccgtcgtcggagctggaattcctgggccacattcatctccagggggcagaggcaggactcagtatcatcatcctgaggtccgcagtcgcccaacttccatgggctgctgggtgtccggtgccggaactgcacctcagcaccaacctggttatccggggtctcccactccatacgcagctgcccggccaacttggacaccttgatgtctcccagaggaggctcatatttaactgagttgtagagctgcagggtcacctcaggagacttctctgtctggttcctggcccaggattccacccagagtgtgacagtgtacagcacagacaccccagcctggtcggagaactgcagcctggtggctgagccggcggcgaagtagcagcagcgcccggagctaaggcaacaccgcaggaagtggctgaccccagctgtgggaccctcatactgccaggagcactcgtaacgatcactggatatccgatagcatctcaggtccctagggcccgaggccgagcctgagtctgcatccggatatggcgggtcctgaaaacagcactcactggttctgcaggcagcgccctgcctggacagcaggaagaggaagaggagggggaccacccaggtcaccagcggctccatcggatccacgtagagccccacagccccaggggagcctctctgccacctgcgaggttcagccacc

SEQ ID NO: 50 - DNA, 4040 bp, Homo sapiens
tgcagagcacagagaaaggacatctgcgaggaaagttccctgatggctgtcaacaaagtgccacgtctctatggctgtgaacgctgagcacacgattttatcgcgcctatcatatcttggtgcataaacgcacctcacctcggtcaacccttgctccgtcttatgagacaggctttattatccgcattttatatgaggggaaactgacggtggagagagaattatcttgctcaaggcgacacagcagagcccacaggtggcagaatcccacccgagcccgcttcgacccgcggggtggaaaccacgggcgcccgcccggctgcgcttccagagctgaactgagaagcgagtcctctccgccctgcggccaccgcccagccccgacccccgccccggcccgatcctcactcgccgccagctccccgcgcccaccccggagttggtggcgcagaggcgggaggcggaggcgggagggcgggcgctggcaccgggaacgcccgagcgccggcagagagcgcggagagcgcgacacgtgcggcccagagcaccggggccacccggtccccgcaggcccgggaccgcgcccgctggcaggcgacacgtggaagaatacggagttctataccagagttgattgttgatggcacatacttttagaggatgctcattggcatttatgtttataatcacgtggctgttgattaaagcaaaaatagatgcgtgcaagagaggcgatgtgactgtgaagccttcccatgtaattttacttggatccactgtcaatattacatgctctttgaagcccagacaaggctgctttcactattccagacgtaacaagttaatcctgtacaagtttgacagaagaatcaattttcaccatggccactccctcaattctcaagtcacaggtcttccccttggtacaaccttgtttgtctgcaaactggcctgtatcaatagtgatgaaattcaaatatgtggagcagagatcttcgttggtgttgctccagaacagcctcaaaatttatcctgcatacagaagggagaacaggggactgtggcctgcacctgggaaagaggacgagacacccacttatacactgagtatactctacagctaagtggaccaaaaaatttaacctggcagaagcaatgtaaagacatttattgtgactatttggactttggaatcaacctcacccctgaatcacctgaatccaatttcacagccaaggttactgctgtcaatagtcttggaagctcctcttcacttccatccacattcacattcttggacatagtgaggcctcttcctccgtgggacattagaatcaaatttcaaaaggcttctgtgagcagatgtaccctttattggagagatgagggactggtactgcttaatcgactcagatatcggcccagtaacagcaggctctggaatatggttaatgttacaaaggccaaaggaagacatgatttgctggatctgaaaccatttacagaatatgaatttcagatttcctctaagctacatctttataagggaagttggagtgattggagtgaatcattgagagcacaaacaccagaagaagagcctactgggatgttagatgtctggtacatgaaacggcacattgactacagtagacaacagatttctcttttctggaagaatctgagtgtctcagaggcaagaggaaaaattctccactatcaggtgaccttgcaggagctgacaggagggaaagccatgacacagaacatcacaggacacacctcctggaccacagtcattcctagaaccggaaattgggctgtggctgtgtctgcagcaaattcaaaaggcagttctctgcccactcgtattaacataatgaacctgtgtgaggcagggttgctggctcctcgccaggtctctgcaaactcagagggcatggacaacattctggtgacttggcagcctcccaggaaagatccctctgctgttcaggagtacgtggtggaatggagagagctccatccagggggtgacacacaggtccctctaaactggctacggagtcgaccctacaatgtgtctgctctgatttcagagaacataaaatcctacatctgttatgaaatccgtgtgtatgcactctcaggggatcaaggaggatgcagctccatcctgggtaactctaagcacaaagcaccactgagtggcccccacattaatgccatcacagaggaaaaggggagcattttaatttcatggaacagcattccagtccaggagcaaatgggctgcctcctccattataggatatactggaaggaacgggactccaactcccagcctcagctctgtgaaattccctacagagtctcccaaaattcacatccaataaacagcctgcagccccgagtgacatatgtcctgtggatgacagctctgacagctgctggtgaaagttcccacggaaatgagagggaattttgtctgcaaggtaaagccaattggatggcgtttgtggcaccaagcatttgcattgctatcatcatggtgggcattttctcaacgcattacttccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagcccgaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctgagtggtgaggcttcaagccttaaagtcagtgtgccctcaaccagcacagcctgccccaattcccccagcccctgctccagcagctgtcatctctgggtgccaccatcggtctggctgcagctagaggacaggcaagccagctctgggggagtcttaggaactgggagttggtcttcactcagatgcctcatcttgcctttcccagggccttaaaattacatccttcactgtgtggacctagagactccaacttgaattcctagtaactttcttggtatgctggccagaaagggaaatgaggaggagagtagaaaccacagctcttagtagtaatggcatacagtctagaggaccattcatgcaatgactatttctaaagcacctgctacacagcaggctgtacacagcagatcagtactgttcaacagaacttcctgagatgatggaaatgttctacctctgcactcactgtccagtacattagacactaggcacattggctgttaatcacttggaatgtgtttagcttgactgaggaattaaattttgattgtaaatttaaatcgccacacatggctagtggctactgtattggagtgcacagctctagatggctcctagattattgagagccttcaaaacaaatcaacctagttctatagatgaagacataaaagacactggtaaacaccaaggtaaaagggcccccaaggtggtcatgactggtctcatttgcagaagtctaagaatgtacctttttctggccgggcgtggtagctcatgcctgtaatcccagcactttgggaggctga

SEQ ID NO: 51 - DNA, 4040 bp, Homo sapiens
tcagcctcccaaagtgctgggattacaggcatgagctaccacgcccggccagaaaaaggtacattcttagacttctgcaaatgagaccagtcatgaccaccttgggggcccttttaccttggtgtttaccagtgtcttttatgtcttcatctatagaactaggttgatttgttttgaaggctctcaataatctaggagccatctagagctgtgcactccaatacagtagccactagccatgtgtggcgatttaaatttacaatcaaaatttaattcctcagtcaagctaaacacattccaagtgattaacagccaatgtgcctagtgtctaatgtactggacagtgagtgcagaggtagaacatttccatcatctcaggaagttctgttgaacagtactgatctgctgtgtacagcctgctgtgtagcaggtgctttagaaatagtcattgcatgaatggtcctctagactgtatgccattactactaagagctgtggtttctactctcctcctcatttccctttctggccagcataccaagaaagttactaggaattcaagttggagtctctaggtccacacagtgaaggatgtaattttaaggccctgggaaaggcaagatgaggcatctgagtgaagaccaactcccagttcctaagactcccccagagctggcttgcctgtcctctagctgcagccagaccgatggtggcacccagagatgacagctgctggagcaggggctgggggaattggggcaggctgtgctggttgagggcacactgactttaaggcttgaagcctcaccactcagagcatgagggagtcacacctcatctttaactgatccagagtcagcttatcaccacaggagaaggtgagtgggtgaagagaacttgaggggaaaacagaaagggagatgtgctgaggctccagttcttccagagagtcagcgagaggtgcctcatgtgaggggaggtcatctatgttggagggtaagtagccatcatgggtgggaaggtcacctgctgggagcaccgtccagggacaggctgggttttcgggctttgggtcggagcccctgctctccagcaccttgtacagatccaccagttgtctgctctcagcttggagagctcttggaggtggtgggcttgaggcactgtgcatcatgtctttctcagaggcctgatgaccttggattcctttttccctttgtggccagttggagcaggggggatgtctgaaaactggggtcacttgatgaaggacttcactgatgaccagcggttcaggatcttcaggcgtgggccagtctatcaggagcctgtccaagggcagctgtgtcttctcctctgcaatgggatatttcttagcgcaagtgctatttgctggatctggaatttctctgctacaccactgaggtctgagggctgctaggagaacaaacaccttttgctggaagtaatgcgttgagaaaatgcccaccatgatgatagcaatgcaaatgcttggtgccacaaacgccatccaattggctttaccttgcagacaaaattccctctcatttccgtgggaactttcaccagcagctgtcagagctgtcatccacaggacatatgtcactcggggctgcaggctgtttattggatgtgaattttgggagactctgtagggaatttcacagagctgaggctgggagttggagtcccgttccttccagtatatcctataatggaggaggcagcccatttgctcctggactggaatgctgttccatgaaattaaaatgctccccttttcctctgtgatggcattaatgtgggggccactcagtggtgctttgtgcttagagttacccaggatggagctgcatcctccttgatcccctgagagtgcatacacacggatttcataacagatgtaggattttatgttctctgaaatcagagcagacacattgtagggtcgactccgtagccagtttagagggacctgtgtgtcaccccctggatggagctctctccattccaccacgtactcctgaacagcagagggatctttcctgggaggctgccaagtcaccagaatgttgtccatgccctctgagtttgcagagacctggcgaggagccagcaaccctgcctcacacaggttcattatgttaatacgagtgggcagagaactgccttttgaatttgctgcagacacagccacagcccaatttccggttctaggaatgactgtggtccaggaggtgtgtcctgtgatgttctgtgtcatggctttccctcctgtcagctcctgcaaggtcacctgatagtggagaatttttcctcttgcctctgagacactcagattcttccagaaaagagaaatctgttgtctactgtagtcaatgtgccgtttcatgtaccagacatctaacatcccagtaggctcttcttctggtgtttgtgctctcaatgattcactccaatcactccaacttcccttataaagatgtagcttagaggaaatctgaaattcatattctgtaaatggtttcagatccagcaaatcatgtcttcctttggcctttgtaacattaaccatattccagagcctgctgttactgggccgatatctgagtcgattaagcagtaccagtccctcatctctccaataaagggtacatctgctcacagaagccttttgaaatttgattctaatgtcccacggaggaagaggcctcactatgtccaagaatgtgaatgtggatggaagtgaagaggagcttccaagactattgacagcagtaaccttggctgtgaaattggattcaggtgattcaggggtgaggttgattccaaagtccaaatagtcacaataaatgtctttacattgcttctgccaggttaaattttttggtccacttagctgtagagtatactcagtgtataagtgggtgtctcgtcctctttcccaggtgcaggccacagtcccctgttctcccttctgtatgcaggataaattttgaggctgttctggagcaacaccaacgaagatctctgctccacatatttgaatttcatcactattgatacaggccagtttgcagacaaacaaggttgtaccaaggggaagacctgtgacttgagaattgagggagtggccatggtgaaaattgattcttctgtcaaacttgtacaggattaacttgttacgtctggaatagtgaaagcagccttgtctgggcttcaaagagcatgtaatattgacagtggatccaagtaaaattacatgggaaggcttcacagtcacatcgcctctcttgcacgcatctatttttgctttaatcaacagccacgtgattataaacataaatgccaatgagcatcctctaaaagtatgtgccatcaacaatcaactctggtatagaactccgtattcttccacgtgtcgcctgccagcgggcgcggtcccgggcctgcggggaccgggtggccccggtgctctgggccgcacgtgtcgcgctctccgcgctctctgccggcgctcgggcgttcccggtgccagcgcccgccctcccgcctccgcctcccgcctctgcgccaccaactccggggtgggcgcggggagctggcggcgagtgaggatcgggccggggcgggggtcggggctgggcggtggccgcagggcggagaggactcgcttctcagttcagctctggaagcgcagccgggcgggcgcccgtggtttccaccccgcgggtcgaagcgggctcgggtgggattctgccacctgtgggctctgctgtgtcgccttgagcaagataattctctctccaccgtcagtttcccctcatataaaatgcggataataaagcctgtctcataagacggagcaagggttgaccgaggtgaggtgcgtttatgcaccaagatatgataggcgcgataaaatcgtgtgctcagcgttcacagccatagagacgtggcactttgttgacagccatcagggaactttcctcgcagatgtcctttctctgtgctctgca

SEQ ID NO: 52 - DNA, 601 bp, Homo sapiens
ggcttagaatgagggcccacactaagagaaaacagataaggcaacttacaaggctgttgaccagctttgtaaactgttcttcaagaaacaatcccaagcttttagctactataccatttttgatatttaatgagctgacgattaaaaatacttgtttgttgcctgttacttgaaaactataaatgccaatgcagtggactcagggtgccacattcttagttaattaagcacccccatttgctgcactaacgggtagcttttaatttgttccaaagggagctaagcagcaagccccggctgyctggagtgctgctatgaaattcctttggttggtgctctatggtcagtagggacggaagcagcaaatggcaacataatggagaaaacatctttaactcaggaagggaggggaggataacccttaagtgctctaaatgaataaaattcaaaagctaaaaacccacaccatgctccagccatatatggcaaagaagcaaaggcctggatacagagaggccggcagcggagcaggatccacaggcagcagctaggggcagccatgaaggtgatcagacagccaacttcctgaggggcctttggg

SEQ ID NO: 53 - DNA, 601 bp, Homo sapiens
taaccagaaaagattaaaagcagtgattaattcttttttaaaggaaagtccaaaggttctggctaaactgactttaaactaagaatcggggagctcattgctttcgctgctgcggccacagccatgagtatgctcaggtttcagatgaggctcgcctttagtgtcctctgctgtggcaagaagaaggtctggttggaccccaataagaccaatgaaatcgccaatgccaacttccatcagcagatccagaagctgatcaaagatgggctgatcatctacaaacctgtgactttccattccygggcttgatgccgggaaaacaccttggcccgctggaagggcaggcacatgggcataagtaagcgaaagggtacagccaatgcccaaatgccagggaacgtaacttggatgaggagaatgcggattctgtgctggctgctgagaagatactgtgaatctaagaagattgatcaccacacatatcacagcctgtacctgaaggtgaaggggaatgtgttcaaaaacaagtggattctcatggaacacatcctcaagctgaaggcagacaaggcccacaagaagctgcaggctgaccaggcta

SEQ ID NO: 54 - DNA, 707 bp, Homo sapiens
ttaaactttttcctttgtttaaattttttattttgaatttttgtgtaattttgaactgaatgtgaatttgagtgtgaatattatatatacaaatatgtgtatattatttatggggtacatgcaatgaaaatttccttttttttttttttttgagacggggtcttgctctgtctcccaggctgtagtgcagtggcatgatctcagctcaatgcagcctctgcctcctggttccagtgattctcctgcctcagcctcctgggtagctgggagtacaggcacgtgccaccacgccaggctaatttttgtatttttagtagagacagggtttcaccatgttggccaggctggccttgaactcctgacctcaggtgatccgcccacctcagccttcctccaaaagtgctaggattacaggtgtgagccacctcgcctggctgaaaacctttttgattacacaaatctctgagcaaataattatactgctagatcatttaaaatagctcctcyatattaattcctaagttttctgctgttattcaaactcaactcctggttttacaggaaaaatagaaaacatattattaggaccatttgaatcaaatgttttcatatgtttaaaacttaaaaaaaaaaatctggccctggaaaaaagtactgaaaagactgctagttcactgtcaggcttctgcataaagataccgagccgg

SEQ ID NO: 55 - DNA, 601 bp, Homo sapiens
gggacagaagcgcagtgcttttggcaccttgcccaagtcacaccctacataggatatcctcaaggaagcaacaggtggtagagagcacaggacagagaaactgaggttggttgatctggtgagtgaggtgcctagattggaggggcagattctggagagaaggctggcaggctctgagaggaggatgtggatgagtgactccaaagtgagctgggagatggggctcaggcctggatgccagtcacccacaattataggtgactcacatccagacagtgactgacaaaggggacctatctcracactctcatagcctcaggctcacctatgttgtttactagttggtccatggctggagaggaggctagtgaactgcattaacatggtcattctgagtaactcactcaggactggctagcaccttcctctatggcttcacacaggtatacaaggctacatctctacaaggctggtaattgacaaagctttacccccaaaggcggtctggtagagagactgtgggcgtggctgacaaaggagagaaaaatcagacatgtgtggccccttagaggacttcaccttctcagaccacttttaccta

SEQ ID NO: 56 - DNA, 845 bp, Homo sapiens
caacaaggggcaggccagggggtgctgccaagagaaagagagactgggagtacctgggtcctccaggatggcgcctgggaagcctcatagctcctactattcagttcccagaattaaaatatttggcttttaacctcaggagccactaaaaaaaacaggtatcattatatatctgtttcttgggacagggtctcgctctgttgcccaggctagagtgcagtggcacagtcatagctcactgcaggctcgacttcccaggcttaggccatcctcccacttcagccccctgaggtagctggggactacaggcatgcaccaccatgcctggctaattttaaaaaaaaattttttttgtagtcatggggtctcactatgttgcctaggctggtcttgaactcctaagctcaagcattcctcctgcctcagcctccctctcatgctgggattacaggcatgagacactgcatctggcccataattctattttcaatgataggakagtaaattcagggtcataaaggaagcctgaagtataagtaaggatataggtagggcttggtcatttacaaaagtcaccatgagacagccttctgaggagttggaaatgatccatagcttggtctgggtgaggattacacagttatatacttccataaaaattcactgagatgtacacttaagatttatgtactttacttttgtaaattttacatcgataaaaacttttaaaaaaaatcagctctgcctgtgcttaattggtctactaggtgcacttggctgcaagagccagttctgggcactctgctaactaaggtagcgctctgctaaccagggtgacccctgca

SEQ ID NO: 57 - DNA, 201 bp, Homo sapiens
gacacagcacacaaagagataccttgttgcttcttgcggcacatcacagtgaagagcgtcgcgaatgccgatatgactaccagtggcactgtgatggccayggctctgatgggcccggcccacggggagtgcactgggaaatttcaaagggaaagtttttaaacttaagcagtgtaaaggttcaaagagaaacaacttttt

SEQ ID NO: 58 - DNA, 601 bp, Homo sapiens
tactagagattttaaaccatggttatatatggaccatatgattacataaaaatatttgtttgaaatatttaatgaagagatcaaaaatcaattatatataaactttttttaaaagcatgttaagaaccatataaaaatataactgtgcacaccttcaatagcttgaaattttagtgttgtaaacagttggaattctatatgaactgggttcattctatagcattaatagggctttcttgtgttatgcatacctcaattatataatcccctggagaaacattttgaaggaggcagctggtcrtctctgtagtttgctcctgcaacagaccaaagaacactttaaaaactcacttcttgggcaaaggtcttcaggacacctcttctcccccagagctgaaggagacttgccaccagtctcaaccacagttttcagaaaatgtgattagatcgaaaagcaactgtgtttatctgccacatgttatctgaacttccctttgataaaaggcccaggacagcccaggacccttgccatagtctatgcttagctggtggcaccctcagcaggaatatttgatttgaagacaaagaaactggtttgaac

SEQ ID NO: 59 - DNA, 808 bp, Homo sapiens
ctgctcaggaggctgaggcatgagaatcgcttgaacccaggaggtggaggttgcagtgagcagagatcacgccactacactccagcctgggtgacagagtgagactctatctcaaaaaagaaaaagaaaagaaaagaaaaaagaactgatagatggggtactgcggttggtgcccaggcctggtcagcgatggccaaccaactgcagactgcagatccctggcccatctggaacccagattcacacagattcaccttgtgtgaatttcacacagattcaccttgtgtgaatttcacacagattcactctgtgtgaaattttaaaaataactgccattattaaaaaatgtttacacaaaaagcacaaatttctcactattcttggaaatcaatttgctaccttcagtgggaattcctcatggccacyatatttggaattaaggagtcaaggctcccctcagactagacatgagctatccagtttgccatactgtccaccattccctagcaccctgcactggggcaactttgtttatgtgttccttgctgggccatactggcagctatgtccttaacaacacatgcagataaaccccaaggaacacagggaaagtctccttacacaacttttctgaacactatggaagacacttgacctggtcaagctcccaaatacttgagtggcctaattgagactgagctgctttagctgttgacaaacctgaacaggttcagaaaattcgtcaaccccaagactgcgaatccttgataagcatggctatgtaatctcttagatgactcaaagggct

SEQ ID NO: 60 - DNA, 704 bp, Homo sapiens
aggtcccctcccctggtgggctgcttgccgcatcacactgtgtgttcctgacacatcggtctgactgctactcacttgaggggaacatggtaattcattcccacaatttgagtagcatggcacttttagggatgtagatgcaaaatggtagttccaattccacagaaaatcagagatcataagaccccgtggccatccacrccgggtcatggagctcagaagcaggagaaaacatcccaggtattagttcttacatgcattttaaagaccttttggctgcgcgtggtggctcacacctgtaatctcagcactttgggaggccgaggcaggtggatcacctgaagtcaggagtttgagaccagcctgaccaacatggtgaaaccccatctctactaaaaatacaaaaaattaactgggtgtaatggcgggcacctgtaatcccagctacttgggaggctgaggcgggagaatcgcttgaacccgggaggcggaggttgcagtgagccgagatcgcgccatcgtactccatcctgggtgacaagaacaaaactccatctcaaacaaacaaacaaacaaaaacctttcattcctggaattcctcaagtgcacagcttctcagcacatgcatcacagatgctctataaggctgtccttagctcaacgcccaaataatacttcagctgctctagagccctcagaaacca

SEQ ID NO: 61 - DNA, 801 bp, Homo sapiens
tgaaattagtggacacaccttgaagttttacaacctaggtagattatagttgcccttgcagtgtttcctcctttgagatttacccaattaaataaaaagaagaagaaaagacacatactcgaagactattgcttttattccacccttcaagcccattaaagttattggtttactttagtcatctctaaacaagggaagaaactccgttgggaaatttggcaagcagagtgcaaaatgtaagaaactgacactctttccttttggtttaagttccccaggtcacatcaaaagcattccaacatgacacatggaattctgggctaacagttgcttccatctctacagggcaccttacttctggtaagaaaatacaacttaggctttttgagtagtcttttagkaattgcccattttaacccatcatactgaaaaaatcacatcaggtgttaagtttctggacaataagatatgccttatgtcttccataggaaaataatagacaaagtacaaagatctgcttaaaactgaatgtaagaagtggcttaggtggattttgccggcttttgcaatagattgtatacattttttaaaatttttatttattttattttattttttgagacgaagccttgttctgtcacccaggctggagtgcaatggtgcaatctcggctcactgcaacctccgcctcccaggttcaagcgattctgctgcctcagccttctgagtagctgggattacaggcatccgccatcacgcccagctaatttttgcatttttgtagagacgggtttcaccatg

SEQ ID NO: 62 - DNA, 1001 bp, Homo sapiens
ctggggaggtgaaaagctggacacagacgcccctagccccttgggatcaggaacagatggaggccgggtgccctgaagtggagcagggaggactgcctggaggagggacacttgagctagaatgtatagaattacagggcatctccggcagaggccacagcagaggcaaaagtccagaggaggtgctttggggaccaccaagatggacacagtgggtctgggtttctgtggggtgatgggctggggcctcgagcgccaggctaaagggcctggctttctccgagggcggtagggagccatggcaggtgtgtgagctggaggggtaccccagacaaaggtgaagtggtatttctccccttattgaagtcacaacctgacactccctctgaagttccccacatcccaccttccccttgccacatcttggcaagcccacctccttccctccgtctctaggcctgccaagaggccctgtgggccccttatggccccacccagctkccccagaagctcgtctgagataagttcccagggctgccccactttcaggtgccccacagactcgggggcgggtgaagcaggaaggatcgagtcacgagggccccagaaaacatgacgggaggaaccacagcacttccccacctcctcagcttccgccttctcagggagagagaccttcagcccgaggaggagggccgagcttccctccatgtggcaagagaccctaacgtcgctgaggatcatctaaagagggtgttgaaggatgcataggagttagccaggagacaacccatttgttaatttaacaaactcataggcatcactcatcaacaagaacaattttcatcattcaatcaggactttctaatgcctctgagggatcatttgaggatgtttaattattatttttatttgagacagggtctcactctgttacccaggctggagtgcagtgacacaatcatagctcactgcagcctcaacctcccagtttcaagcta